SEQUENCE LISTING (1 sequence)

SEQ ID NO: 1 - protein, 37 aa, synthetic construct
  MOD_RES 11: note = Aib
  MOD_RES 18: note = (1S,28S)-1-amino-7,16,25,30-tetraoxo-9,12,18,21-tet raoxa-6,15,24,29-tetraazaoctatetracontane-1,28,48-tricarbo xylic acid
EGTFTSDYSIXLDKIAQXAFVQWLIAGGPSSGAPPPS

(19) United States
(12) Patent Application Publication
(10) Pub. No.: US 2024/0390464 A1
(43) Pub. Date: Nov. 28, 2024

(54) Treatment of Adults With Overweight or Obesity With or Without Weight-Related Comorbidities

(71) Applicant: Carmot Therapeutics Inc., Berkeley, CA (US)

(72) Inventors: Manu Chakravarthy, Newton, MA (US); Alexandra Steinberg, Alameda, CA (US); Jingtao Wu, Lexington, MA (US); Michael Elliott, Wake Forest, NC (US)

(73) Assignee: Carmot Therapeutics Inc., Berkeley, CA (US)

(21) Appl. No.: 18/670,592

(22) Filed: May 21, 2024

Related U.S. Application Data
(60) Provisional application No. 63/503,492, filed on May 21, 2023.
(60) Provisional application No. 63/511,858, filed on Jul. 3, 2023.
(60) Provisional application No. 63/590,395, filed on Oct. 13, 2023.
(60) Provisional application No. 63/648,158, filed on May 15, 2024.

(51) Int. Cl. A61K 38/26; A61K 9/00; A61K 38/22; A61P 3/04; A61P 3/10

(52) U.S. Cl. CPC A61K 38/26; A61K 9/0019; A61K 38/22; A61P 3/04; A61P 3/10

(57) ABSTRACT

The present disclosure provides methods of treating overweight or obese patients with or without type 2 diabetes mellitus. The treatment comprises administration of a dual GLP-1 receptor and GIP receptor agonist.

### CROSS-REFERENCE TO RELATED APPLICATIONS

The present application claims priority from U.S. Applications 63/503,492, filed May 21, 2023; 63/511,858, filed Jul. 3, 2023; 63/590,395, filed Oct. 13, 2023; and 63/648,158, filed May 15, 2024. The contents of the priority applications are incorporated by reference herein in their entirety.

### SEQUENCE LISTING

The instant application contains a Sequence Listing that has been submitted electronically in XML format and is hereby incorporated by reference in its entirety. Said XML copy, created on May 21, 2024, is named 124921US005.xml and is 2,890 bytes in size.

### BACKGROUND OF THE INVENTION

Obesity is the most prevalent chronic disease worldwide, affecting as many as 650 million adults. Obesity is associated with numerous complications including cardiovascular disease and type 2 diabetes mellitus (T2DM or T2D), thereby posing a substantial health and economic burden on patients and society.

T2DM results from the inability of insulin to properly control blood glucose. This state of insulin resistance causes the pancreas to secrete more insulin, resulting in hyperinsulinemia; yet blood glucose levels remain abnormally elevated. Over time, this condition results in beta cell failure and insufficient insulin production, cardiovascular damage, and a significant increase in the risk of heart attacks, strokes, neuropathy, limb amputation, blindness, and kidney failure.

Obesity is a major risk factor for T2DM. About 80% of T2DM patients are overweight or obese. The relationship between obesity and T2DM is well established, and the global obesity epidemic largely explains the multifold increase in the prevalence rate of T2DM in recent years. Obesity-related prediabetes increases the risk of developing T2DM by several folds. Once obesity is present, it is often difficult to lower weight due to the complex compensatory and homeostatic mechanisms that counter-regulate body weight.

It has become increasingly clear that effective diabetes treatments must address both insulin sensitivity and obesity (Khaodhiar et al., Curr Diab Rep. (2009) 9(5):348-54). Lifestyle intervention in the form of dietary, behavioral, and exercise counselling is traditionally the primary treatment for T2DM and obesity. In fact, multiple studies have shown that severe restriction of caloric intake for at least six months can reverse T2DM in many patients. But such treatment regimens are rarely successful due to poor compliance. The most widely prescribed anti-diabetic medications such as metformin, sulfonyl urea, and dipeptidyl peptidase 4 (DPP4) inhibitors do not produce significant weight loss and may even cause weight gain (Azimova et al., Ochsner J. (2014) 14(4):616-32). Sodium-glucose cotransporter 2 inhibitors (e.g., empagliflozin and canagliflozin) produce 2% to 4% weight loss and are among the few anti-diabetic drugs that have shown improved cardiovascular outcomes. Several GLP-1 mimetics have also been approved for the treatment of T2DM. These drugs lower blood glucose and glycated hemoglobin (HbA1c) and produce modest weight loss, but their efficacy is limited by gastrointestinal (GI) side effects such as nausea, vomiting, and diarrhea (Marin-Penalver et al., World J Diabetes (2016) 7(17):354-95).

Thus, there remains an acute need for developing a safe, effective, and well-tolerated therapy for chronic weight management and glycemic control in overweight or obese people living with or without weight-related comorbidities such as T2DM.

### SUMMARY OF THE INVENTION

The present disclosure provides a method of weight management (inducing weight loss, or treating obesity or overweight), improving glycemic control and/or glucose homeostasis, and/or increasing insulin sensitivity in an overweight or obese adult patient in need thereof, comprising administering by subcutaneous injection to the patient a compound of Formula I or Formula II, or a pharmaceutically acceptable salt or ester thereof:

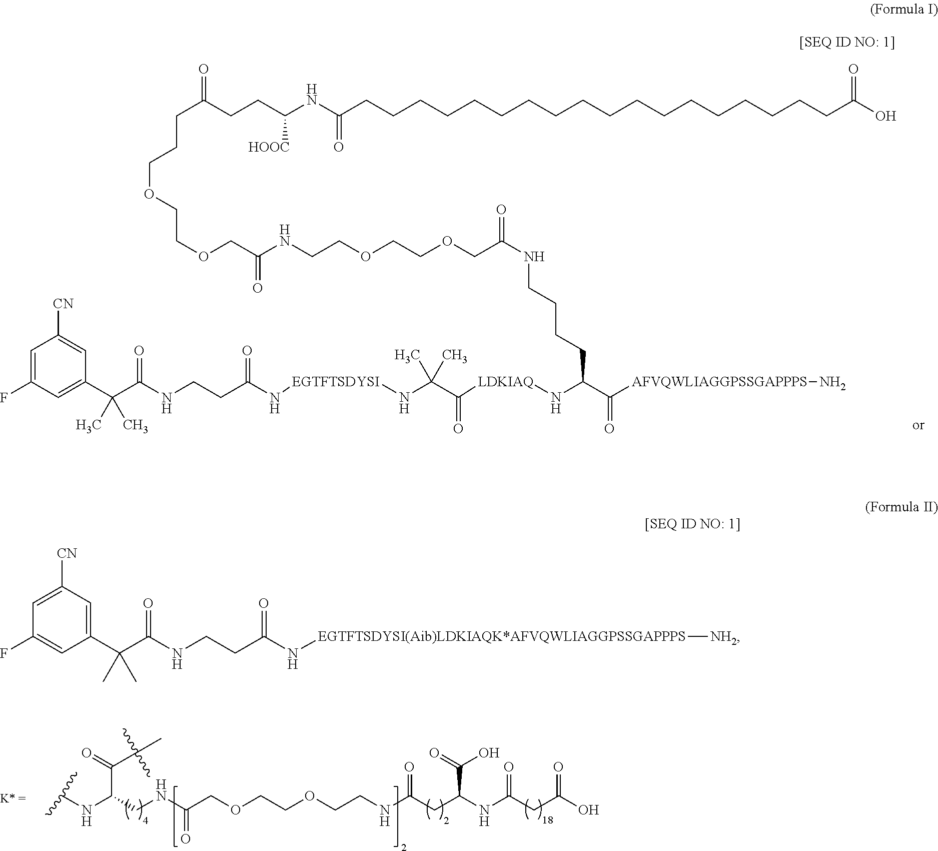

wherein the compound is administered at a flat dose (i.e., fixed dose, independent of body weight) of about 2.0 mg to about 25 mg (e.g., about 5 mg to about 24 mg, or about 5 mg to about 22 mg).

In some embodiments, the patient has a BMI of ≥25 kg/m², ≥27 kg/m², ≥30 kg/m², ≥35 kg/m², or ≥40 kg/m². In some embodiments, the patient also has one or more weight-related comorbidities (e.g., diabetes, prediabetes, hypertension, or dyslipidemia). In some embodiments, the patient is obese with or without type 2 diabetes mellitus (T2DM). In some embodiments, the patient is obese and prediabetic.

In some embodiments, the compound is administered once every week, once every two weeks, or once every month.

In some embodiments, the compound is administered at about 5, 6, 7.5, 8, 12, 16, 17, 20, 22, or 24 mg per dose.

In some embodiments, the compound's dose is up-titrated over a period of 3 weeks to 30 weeks (e.g., one, two, three, four, five, or six months). In some embodiments, the starting dose for the up-titration is about 2 mg, about 4 mg, about 5 mg, or about 8 mg. In some embodiments, the maximum dose for the up-titration is about 22 or 24 mg.

In some embodiments, the patient receives an additional therapy for overweight or obesity, optionally wherein the additional therapy is diet therapy or exercise therapy.

The therapy herein may result in reduction of visceral adiposity and/or HbA1c level in the treated patient.

Also provided in the present disclosure is a compound for use in the therapy, wherein the compound is of Formula I or Formula II, or a pharmaceutically acceptable salt or ester thereof. The present disclosure also provides use of the compound for the manufacture of a medicament for use in the present therapy.

In another aspect, the present disclosure provides an article of manufacture (e.g., a kit) for use in the therapy described herein, wherein the article of manufacture comprises one or more dose units of the compound. The article of manufacture may contain a syringe or an injector, optionally a single-use syringe or injector.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1A is a graph showing Cₘₐₓ (ng/mL; maximum observed concentration) of the tested doses (0.5, 2, 4, 6, and 8 mg) in Cohorts 1-5.

FIG. 1B is a graph showing AUC₀₋ₜ (h*ng/mL; area under the concentration-time curve from time zero to the time of the last measurable concentration) over the tested doses (0.5, 2, 4, 6, and 8 mg) in Cohorts 1-5.

FIG. 1C is a table showing preliminary PK parameters from Cohorts 1-5. CL/F: oral clearance; MRTₗₐₛₜ: mean residence time from the time of dosing to the time of the last measurable concentration; t_{1/2}: apparent (estimated) terminal elimination half-life (shown as range of minimum to maximum); Vz/F: terminal volume of distribution/bioavailability. All values are mean±standard deviation.

FIG. 2A is a graph showing mean body weight (BW) change (%) from baseline as measured over a period of 43 days following once weekly (QW) administration of placebo or CT-388 for 4 weeks following the regimen: 5/5/5/7.5 mg (cohort 6 or “C6”); 5/5/8/12 mg (C7); or 5/8/12/12 mg (C8).

FIG. 2B is a graph showing mean change in percentage body weight (%) from baseline as measured on Day 29 following QW administration of placebo (Pbo) or CT-388 for 4 weeks following the regimen: 5/5/5/7.5 mg (C6); 5/5/8/12 mg (C7); or 5/8/12/12 mg (C8). *: p<0.001 vs. placebo.

FIG. 3 is a graph showing change in waist circumference (cm) as measured on Day 29 following QW administration of placebo (Plb) or CT-388 for 4 weeks following the regimen: 5/5/5/7.5 mg; 5/5/8/12 mg; or 5/8/12/12 mg.

FIG. 4 is a graph showing change in HOMA-IR (Homeostatic Model Assessment for Insulin Resistance) as measured on Day 29 following QW administration of placebo or CT-388 for 4 weeks following the regimen: 5/5/5/7.5 mg; 5/5/8/12 mg; or 5/8/12/12 mg.

FIG. 5 is a set of graphs showing glucose response from baseline to Day 23 in Cohorts 6-8 vs. placebo.

FIG. 6 is a set of graphs showing insulin response from baseline to Day 23 in Cohorts 6-8 vs. placebo.

FIGS. 7A and 7B are related to the PK parameters of CT-388. FIG. 7A is a table showing preliminary PK parameters from Cohorts 6-8. Tₘₐₓ: time to maximum concentration; Cₘₐₓ: maximum observed concentration; AUC₀₋ₜ: area under the concentration-time curve from time zero to the time of the last measurable concentration; Cₘᵢₙ: minimum observed concentration. All values are mean±standard deviation. FIG. 7B is a graph showing the preliminary PK parameters.

FIG. 8 shows arm 3 of the seamless phase 1/2 design of clinical trial CT-388-101, a first in human Phase 1, double-blinded, placebo controlled, single center study.

FIG. 9 shows the design of the MD part of the CT-388-101 clinical trial. Cohorts 11 and 12 are obese patients without T2DM cohorts, and cohort 13 is obese patients with T2DM.

FIG. 10 shows the subject dispositions in the MD part of the CT-388-101 clinical trial. *Adjustments included skip doses or dose down titrations; ** Food poisoning; * Drug holiday at W23 (bi-weekly dosing) due to BMI<23 kg/m².

FIG. 11 shows the baseline adjusted LS mean % change in body weight at weeks 12 and 24. *p<0.05; ** p<0.01; *** p<0.001 when comparing CT-868 with placebo after accounting for baseline effect in MMRM models (with baseline weight as the covariate).

FIG. 12 shows a sensitivity analysis of the LS mean % change in body weight at weeks 12 and 24, adjusted for baseline weight and sex. * p<0.05; ** p<0.01; *** p<0.001 when comparing CT-868 with placebo after accounting for baseline effect in MMRM models (with baseline weight and sex as the covariates).

FIG. 13 shows an MMRM model of LS mean±SE of % change in body weight in patients over 24 weeks.

FIG. 14 shows an MMRM model of LS Mean±SE of % change in body weight over 24 Weeks in cohort 12 by actual final dose (22 mg vs 12 mg). *** p<0.001 when comparing CT-388 with Placebo after accounting for baseline effect in MMRM models (baseline weight as the covariate).

FIG. 15 shows the percentage of participants meeting specified weight loss thresholds at Week 24.

FIG. 16 shows the baseline adjusted mean change in waist circumference (WC) to weeks 12 and 24. p<0.05; ** p<0.01; *** p<0.001 when comparing CT-868 with placebo after accounting for baseline effect in MMRM models (with baseline waist as the covariate).

FIGS. 17A and 17B show measures of glycemic control during the treatment period. FIG. 17A shows the mean (SD) change from baseline in HbA1c and FIG. 17B shows the mean (SD) of HbA1c (%).

FIG. 18 shows the observed shift in glycemic status in trial participants.

FIG. 19 is a study schema showing randomized treatment groups receiving CT-388 in 2, 4, 8, 12, 16, 24 mg or placebo across six arms in participants with obesity or overweight with at least one weight-related comorbidity. *The goal is to administer the indicated doses and up-titrate per the specified titration path. However, based on individual's tolerability, there is flexibility (i.e., doses may be held at the same dose, down-dosed, or a dosing holiday may be provided) during this up-titration period with the goal to reach the assigned target dose by week 27, the last up-titration time point. The dotted lines indicate potential final dose that can be considered for safety reasons during the up-titration period. ** No further dose up-titration is allowed past week 28; however, a dosing holiday or dose down-titration may be performed if required (i.e., GI-related AE, significant weight loss of close to BMI≤22 kg/m²) reasons. *** Week 27: Last up-titration point.

FIG. 20 is a flowchart showing the management of participants with gastrointestinal (GI) symptoms.

FIG. 21 is a study schema showing randomized treatment groups receiving CT-388 at a dose of placebo or 2, 4, 8, 12, 16, 24 mg across six arms in participants who are overweight or obese with type 2 diabetes mellitus.

### DETAILED DESCRIPTION OF THE INVENTION

The present disclosure provides a therapy (e.g., monotherapy or adjunctive therapy) for weight management in human adults who are overweight or obese with or without weight-related comorbidities such as T2DM. The therapy comprises administration of a unimolecular agonist for both glucagon-like peptide-1 (GLP-1) receptor (GLP-1R) and glucose-dependent insulinotropic polypeptide (GIP) receptor (GIPR). The therapy herein may induce weight loss and improve glucose homeostasis (both in fasted and postprandial conditions) in the treated subjects.

### I. Dual GLP-1R/GIPR Agonist

GLP-1 and GIP are primary incretins released from the gastrointestinal tract in response to food intake. Both hormones modulate glucose-dependent insulin secretion. GLP-1 also decreases secretion of glucagon, slows gastric emptying, promotes satiety, and reduces food intake. Several GLP-1 mimetics have been approved for the treatment of type 2 diabetes mellitus (T2DM), but their efficacy is limited by gastrointestinal side effects such as nausea, vomiting, and diarrhea.

The primary action of GIP is the stimulation of glucose-dependent insulin secretion. It also modulates the secretion of glucagon in an inverse glucose-dependent manner to protect against hypoglycemia and does not delay gastric emptying. GIP also stimulates glucose uptake in adipocytes and promotes bone strength.

The dual GLP-1R/GIPR agonist used in the present therapy, referred to as “CT-388,” potently activates production of cyclic adenosine monophosphate (cAMP), but has no or minimal activity on the β-arrestin signaling pathways on either GLP-1R or GIPR. That is, the agonist is fully biased towards cAMP activation, as opposed to being partially biased (i.e., with some β-arrestin activity) or unbiased (i.e., with full β-arrestin activity), on both GLP-1R and GIPR. β-arrestin activates kinase signaling pathways, but also causes the GLP-1R and GIPR to be turned off and internalized (Hsia et al., Curr Opin Endocrinol Diabetes Obes. (2017) 24(1):73-9). The present agonist does not cause internalization and consequently, desensitization of either GLP-1R or GIPR, and thus has enhanced signaling efficacy.

CT-388 has the following structural formula:

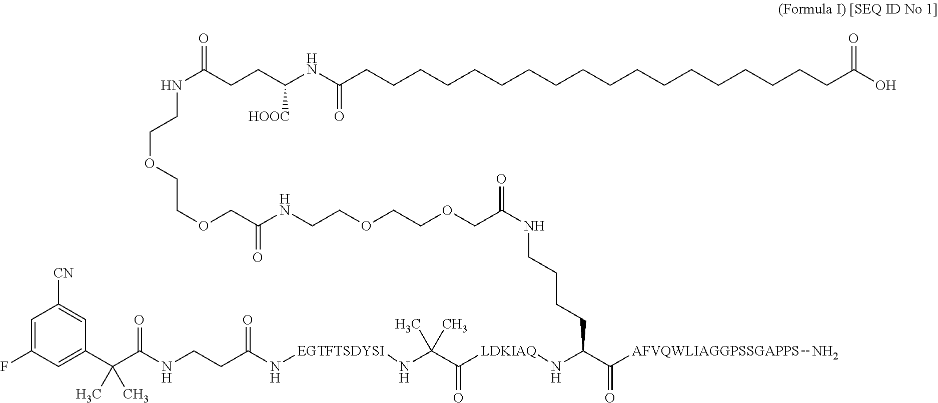

CT-388's structure can also be depicted as follows:

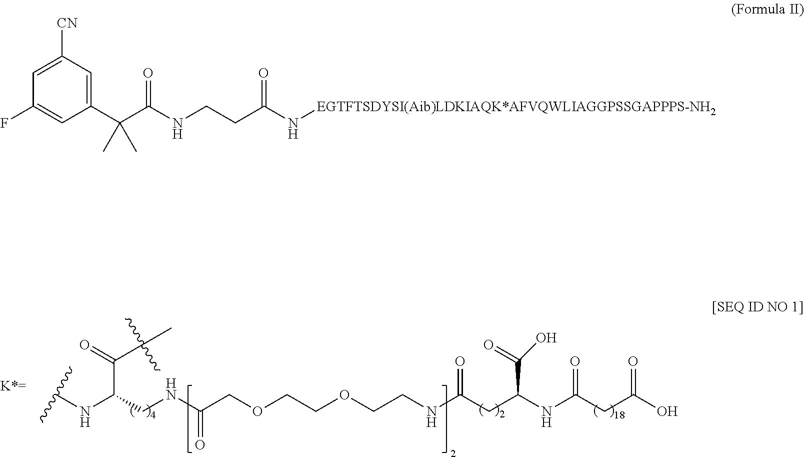

Formula I and Formula II both refer to the same compound.

A pharmaceutically acceptable salt (e.g., sodium or phosphate salt) of the above-depicted compound is also within the meaning of “CT-388” herein. Pharmaceutically acceptable esters of the above-illustrated compound may also be used in the present therapy.

The present disclosure provides pharmaceutical compositions comprising CT-388 and a pharmaceutically acceptable excipient. In some embodiments, the only active pharmaceutical ingredient (API) of the pharmaceutical compositions is the compound of Formula I or II.

In some embodiments, CT-388 is provided as a sterile, lyophilized powder that can be reconstituted as a solution suitable for subcutaneous injection. In some embodiments, CT-388 is provided in a sterile aqueous solution suitable for subcutaneous injection. For example, CT-388 may be provided at a concentration of 20 mg/mL in sodium phosphate buffer comprising mannitol, pH 7.0.

CT-388 may be well tolerated and cause fewer adverse effects than other incretin mimetics. For example, chronic toxicology data show that CT-388 was well tolerated in repeat dosing over 26-weeks and 39-weeks in rats and monkeys, respectively, which exceeds the pharmacologically active dose by 100-fold, and resulted in weight loss up to 10%. Thus, CT-388 can be a potent drug that induces significant weight loss and helps achieve optimal glycemic control, with acceptable tolerability, in overweight or obese adults with or without T2DM or other weight-related comorbidities. CT-388 can potentially be used as a non-invasive alternative to bariatric surgery for weight management.

### II. Patient Populations

In some embodiments, the patient is at least 18 and up to 75 years of age. Exemplary patient populations suitable for being treated by the present methods are further described below.

A. Otherwise Healthy Overweight and Obese Adults and Obese Patients with Type 2 Diabetes Mellitus

The weight management treatment herein may be chronic or for a shorter term as determined by a physician. In some embodiments, the present disclosure provides a CT-388 therapy for weight management in obese or overweight adults with or without weight-related abnormalities. In some embodiments, the adult patients have one or more weight-related (treated or untreated) comorbidities (e.g., prediabetes, T2DM, hypertension, and dyslipidemia). In some embodiments, such adults are prediabetic and the therapy improves glycemic control in these adults; in further embodiments, the prediabetic adults are obese.

In some embodiments, the present disclosure provides a CT-388 therapy for weight management in obese or overweight otherwise healthy adults. An overweight or obese adult who is otherwise healthy lacks clinically significant history or active clinical manifestation(s) of any significant metabolic, proliferative, allergic, dermatological, hepatic, renal, hematological, pulmonary, cardiovascular, gastrointestinal, neurological, or psychiatric disorder.

In some embodiments, the present disclosure provides a CT-388 therapy for weight management and glycemic control in obese or overweight adults with T2DM. In some embodiments, such adults have one or more weight-related or T2DM-related (treated or untreated) comorbidities (e.g., hypertension and dyslipidemia).

In some embodiments, the present disclosure provides a CT-388 therapy for glycemic control in adults with T2DM who are not obese or overweight (e.g., BMI<25 kg/m²).

As used herein, an adult is a human aged 18 or older. In some embodiments, the present therapy treats an adult who is between 18 and 65 years of age, inclusive. In some embodiments, the patient is 75 years of age or less (for example, 65 years of age or less, 66 years of age or less, 67 years of age or less, 68 years of age or less, 69 years of age or less, 70 years of age or less, 71 years of age or less, 72 years of age or less, 73 years of age or less, or 74 years of age or less).

The health conditions of patients to be treated in the present therapies are further described below.

### 1. Overweight and Obesity

Body mass index (BMI) is a simple index of weight-for-height that is commonly used to classify overweight and obesity in adults. It is defined as a person's weight in kilograms divided by the square of his height in meters (kg/m²). For adults, WHO defines overweight as having a BMI of 25 to 29.9 kg/m², and obesity as having BMI≥30 kg/m². Obesity is frequently subdivided into the following categories:
Obesity class I—BMI 30 to 34.9 kg/m²
Obesity class II—BMI 35 to 39.9 kg/m²; and
Obesity class III—BMI≥40 kg/m² (also referred to as severe, extreme, or massive obesity)

In some embodiments, the definitions of overweight and obesity in Asian and South Asian populations may be as follows:
Overweight—BMI 23 to 24.9 kg/m²
Obesity—BMI≥25 kg/m²

In some embodiments, the patient to be treated herein is overweight. In other embodiments, the patient to be treated herein is obese, with class I, II, or III obesity.

### 2. T2DM

T2DM and prediabetes may be defined by the 2022 ADA standards of Medical Care in Diabetes (American Diabetes Association's (ADA), “Standards of Medical Care in Diabetes,” Clin Diabetes (2022) 40(1):10-38). The ADA definitions are shown in Table 1 below.

TABLE 1
Glycemic Status Definitions

| | Definitions* | | |
|---|---|---|---|
| Diagnostic Tests | Normoglycemia | Prediabetes | Diabetes |
| (FPG obtained separately or at | <100 mg/dL | 100-125 mg/dL | ≥126 mg/dL |
| time = 0 minutes during an OGTT | (<5.6 mmol/L) | (5.6-6.9 mmol/L) | (≥7.0 mmol/L) |
| 2-hour glucose | <140 mg/dL | 140-199 mg/dL | ≥200 mg/dL |
| obtained at time = 120 | (<7.8 mmol/L) | (7.8-11.0 mmol/L) | (≥11.1 mmol/L) |
| minutes during an | | | |
| OGTT | | | |
| HbA1c | <5.7% | 5.7%-6.4% | ≥6.5% |
| | (<39 mmol/mol) | (39-47 mmol/mol) | (≥48 mmol/mol) |

*FPG: fasting plasma glucose. OGTT: oral glucose tolerance test. HbA1c: hemoglobin 1C.

To make the diagnosis of prediabetes or diabetes, at least one of the above tests (i.e., FPG, 2-hour post-challenge plasma glucose, or HbA1c) needs to be abnormal (i.e., outside/above the normoglycemic range/values for each corresponding test).

In some embodiments, the patient has type 2 diabetes mellitus (T2DM). In some embodiments, the T2DM patient has a BMI of ≥25 kg/m², ≥27 kg/m², ≥30 kg/m², ≥35 kg/m², ≥40 kg/m², or ≥45 kg/m². In some embodiments, the T2DM patient is not overweight or obese.

### 3. Other Medical Conditions

In some embodiments, the patient treated herein has one or more comorbidities, such as prediabetes (see above), hypertension, and dyslipidemia. Hypertension can be defined as current use of blood pressure lowering agents initiated for hypertension, or with systolic blood pressure (SBP)≥130 mmHg or diastolic blood pressure (DBP)≥80 mmHg. Dyslipidemia can be defined as current use of lipid-lowering agents initiated for dyslipidemia, or with low-density lipoprotein (LDL)≥160 mg/dL (4.1 mmol/L) or triglycerides≥150 mg/dL (1.7 mmol/L), or high-density lipoprotein (HDL)<40 mg/dL (1.0 mmol/L) for men or HDL<50 mg/dL (1.3 mmol/L) for women.

In some embodiments, the patient herein is not pregnant or breastfeeding.

In some embodiments, the patient herein does not have a history of bariatric surgery or gastric banding (including LAP-BAND), history of any gastrointestinal surgery that may induce malabsorption (e.g., bowel resection), or any GI motility disorders, gastroparesis, delayed gastric emptying, inflammatory bowel disease, pancreatitis, malabsorption syndromes, chronic diarrhea, chronic constipation, and clinically significant irritable bowel syndrome.

In some embodiments, the patient herein does not have semi-supine blood pressure systolic>160 mm Hg and/or diastolic>95 mm Hg.

In some embodiments, the patient herein does not have an active or untreated malignancy or patients who have been in remission from a clinically significant malignancy (other than basal or squamous cell skin cancer, in situ carcinomas of the cervix, or in situ prostate cancer) for <5 years.

In some embodiments, the patient herein does not have a personal or family history (first-degree relative) of medullary thyroid carcinoma (MTC), or a genetic condition that predisposes to MTC (i.e., thyroid C-cell hyperplasia, or multiple endocrine neoplasia type 2A or type 2B).

In some embodiments, the patient herein does not have a history of atopy (severe or multiple allergic manifestations) or clinically significant multiple or severe drug allergies, or intolerance to topical corticosteroids, or severe posttreatment hypersensitivity reactions; known allergy to any component of the pharmaceutical composition, GLP-1 analogues, or related compounds.

In some embodiments, the patient herein does not have hormone replacement therapy.

In some embodiments, the patient herein does not concurrently receive other pharmacotherapy for obesity including, but not limited to: Saxenda® (liraglutide), Wegovy® (semaglutide), Mounjaro® (tirzepatide), Xenical® or Alli® (orlistat), Meridia® (sibutramine), Acutrim® (phenylpropanolamine), Sanorex® (mazindol), Apidex® (phentermine), BELVIQ® (lorcaserin), Qsymia® (phentermine/topiramate combination), Contrave® (naltrexone/bupropion), Asenlix® (Clobenzorex), Solucaps® (Mazindol), Redotex® (aloine, atropine, diazepam, norpseudoephedrine, triyodotironine), berberine, resveratrol, other sympathomimetic agents, thyroid hormones (for nonthyroid indications), growth hormone, or any non-herbal supplements/alternative remedies with unknown/unspecified content.

In some embodiments, the patient to be treated herein does not concurrently receive other drugs that target GLP-1R and/or GIPR.

B. Obese or Overweight Patients with at Least One Weight-Related Comorbidity

In some embodiments, the patient is 18 years of age and up to 75 years of age. In some embodiments, the patient is obese as defined as having a BMI of ≥30.0 kg/m². In some embodiments, the patient is overweight as defined as having a BMI≥27.0 and <30.0 kg/m² and has been previously diagnosed with at least one of the following weight-related comorbidities:
a. prediabetes, as defined by the 2024 ADA Standards of Medical Care in Diabetes;
b. hypertension, defined as current use of blood pressure lowering agents initiated for hypertension, or with systolic blood pressure≥130 mm Hg or diastolic blood pressure≥80 mm Hg;
c. dyslipidemia, defined as current use of lipid-lowering agents initiated for dyslipidemia, or with low-density lipoprotein (LDL)≥160 mg/dL (4.1 mmol/L) or triglycerides≥150 mg/dL (1.7 mmol/L), or high-density lipoprotein (HDL)<40 mg/dL (1.0 mmol/L) for men or HDL<50 mg/dL (1.3 mmol/L) for women;
d. obstructive sleep apnea; or
e. cardiovascular disease (for example, ischemic cardiovascular disease, New York Heart Association (NYHA) Functional Class I-II heart failure)

In some embodiments, the patient has a history of ≥1 self-reported unsuccessful diet/exercise effort to lose body weight.

In some embodiments, the patient does not have a prior history or diagnosis of any type of diabetes mellitus (e.g., type 1, type 2, gestational), or does not have a history of ketoacidosis or hyperosmolar state. The patient may not have diabetes mellitus as determined based on fasting glucose or HbA1c.

In some embodiments, the patient has a diagnosis of prediabetes (determined by fasting glucose or HbA1c levels). Such patients may not be on any type of glucose-lowering treatments (pharmacologic or dietary supplements), for example metformin or berberine.

In some embodiments, the patient has not had a prior surgical treatment of any type for obesity. In some embodiments, the patient has not undergone LAP-BAND®, intragastric balloon, duodenal sleeve, resurfacing, liposuction, or abdominoplasty procedures.

In some embodiments, the patient does not have a known clinically significant or active gastric emptying abnormality (e.g., severe gastroparesis or gastric outlet obstruction, intestinal obstruction, or any gastrointestinal (GI) motility disorders); malabsorption, including chronic constipation/diarrhea, celiac disease, inflammatory bowel disease, or bowel resection; or has chronically take drugs that directly affect GI motility (e.g., anticholinergics, 5-hydroxytryptamine [serotonin] antagonists, or opiates).

The patient may not have chronic or acute pancreatitis. In some embodiments, the patient does not have a history of complications from gall stones and/or acute cholecystitis or uncontrolled hypertension (systolic blood pressure≥160 mm Hg and/or diastolic blood pressure≥100 mm Hg).

In some embodiments, the patient does not have congestive heart failure (for example, NYHA Functional Classification III or IV congestive heart failure), myocardial infarction, cerebrovascular accident (stroke), transient ischemic attack, unstable angina, coronary artery bypass graft, or percutaneous coronary intervention. In some embodiments, the patient does not have a personal or family history of long QT syndrome, family history of sudden death in a first-degree relative (parents, sibling, or children) before the age of 40 years, or a personal history of unexplained syncope.

In some embodiments, the patient does not have uncontrolled thyroid disease, defined as active symptoms (e.g., palpitations, lethargy, weight gain/loss) and/or having a thyroid-stimulating hormone (TSH) level outside the normal reference range.

In patients with obesity, the obesity may not be induced by other endocrinologic disorders (e.g., Cushing syndrome, acromegaly, inadequately treated hypothyroidism) or diagnosed monogenetic or syndromic forms of obesity (e.g., Melanocortin 4 Receptor deficiency or Prader-Willi syndrome). In some embodiments, the patient does not have a family or personal history of medullary thyroid carcinoma or a genetic condition that predisposes to medullary thyroid carcinoma (i.e., thyroid C-cell hyperplasia, or multiple endocrine neoplasia Type 2A or Type 2B).

In some embodiments, the patient does not have:
a. alanine aminotransferase (ALT), aspartate aminotransferase (AST), or gamma glutamyl transferase (GGT)>3.0× the upper limit of normal (ULN) for the reference range;
b. alkaline phosphatase (ALP)>1.5×ULN for the reference range;
c. total bilirubin>1×ULN for the reference range;
d. amylase or lipase>2×ULN for the reference range;
e. calcitonin≥20 ng/L;
f. estimated glomerular filtration rate (eGFR)<60 mL/min/1.73 m² by the 2021 CKD-EPI creatinine equation;
g. fasting plasma triglycerides≥500 mg/dL;
h. acute or chronic viral hepatitis (determined based on hepatitis B surface antigen [HbsAg] or presence of HCV RNA);
i. positive HIV antibody screen;
j. hemoglobin value <11 g/dL (men) or <10 g/dL (women);
k. white blood cells (WBC)<the lower limit of normal (LLN) for the reference range; or
l. platelets<LLN for the reference range.
   C. Overweight or Obese Patients with Type 2 Diabetes Mellitus

In some embodiments, the patient is between 18 and 75 years of age. The patient may have a BMI of ≥25.0 kg/m². The patient may also have a diagnosis of T2DM according to, e.g., the World Health Organization classification. The patient's HbA1c levels may be ≥7% and ≤10.5%.

In some embodiments, the patient has managed their T2DM with diet and exercise alone prior to treatment. In some embodiments, the patient has managed their T2DM with stable treatment with metformin, a sulfonylurea, or a SGLT-2 inhibitor as monotherapy or combination therapy. The patient may have a history of ≥one self-reported unsuccessful diet/exercise effort to lose body weight.

In some embodiments, the patient does not have type 1 diabetes mellitus (T1DM) or any other types of diabetes except T2DM. The patient may not have a history of ketosis or hyperosmolar state/coma. In some embodiments, the patient does not have proliferative diabetic retinopathy, diabetic macular edema, or non-proliferative diabetic retinopathy that requires acute treatment based on a dilated fundoscopic examination performed by an ophthalmologist or optometrist between screening and randomization.

The patient may not have clinically significant/active nephropathy or neuropathy (including resting tachycardia, orthostatic hypotension, diabetic diarrhea).

In some embodiments, the patient has not had Lap-Band®, intragastric balloon, duodenal sleeve, resurfacing, liposuction, and/or abdominoplasty procedures.

In some embodiments, the patient does not have a known clinically significant or active gastric emptying abnormality (e.g., severe gastroparesis or gastric outlet obstruction, intestinal obstruction, or any GI motility disorders); malabsorption, including chronic constipation/diarrhea, celiac disease, inflammatory bowel disease, or bowel resection; or has chronically take drugs that directly affect GI motility (e.g., anticholinergics, 5-hydroxytryptamine [serotonin] antagonists, opiates).

The patient may not have chronic or acute pancreatitis. In some embodiments, the patient does not have a history of complications from gall stones and/or acute cholecystitis or uncontrolled hypertension (systolic blood pressure≥160 mm Hg or diastolic blood pressure≥100 mm Hg).

In some embodiments, the patient does not have congestive heart failure (e.g., NYHA Functional Classification III or IV congestive heart failure), myocardial infarction, cerebrovascular accident (stroke), transient ischemic attack, unstable angina, coronary artery bypass graft, or percutaneous coronary intervention. In some embodiments, the patient does not have a personal or family history of long QT syndrome, family history of sudden death in a first-degree relative (parents, sibling, or children) before the age of 40 years, or a personal history of unexplained syncope.

In some embodiments, the patient does not have uncontrolled thyroid disease, defined as active symptoms (e.g., palpitations, lethargy, weight gain/loss) and/or having a thyroid-stimulating hormone (TSH) level outside the normal reference range.

In patients with obesity, the obesity may not be induced by other endocrinologic disorders (e.g., Cushing syndrome, acromegaly, inadequately treated hypothyroidism) or diagnosed monogenetic or syndromic forms of obesity (e.g., Melanocortin 4 Receptor deficiency or Prader-Willi syndrome). In some embodiments, the patient does not have a family or personal history of medullary thyroid carcinoma or a genetic condition that predisposes to medullary thyroid carcinoma (i.e., thyroid C-cell hyperplasia, or multiple endocrine neoplasia Type 2A or Type 2B).

In some embodiments, the patient does not have:
a. alanine aminotransferase (ALT), or aspartate aminotransferase (AST), or gamma glutamyl transferase (GGT)>3.0× the upper limit of normal (ULN) for the reference range;
b. alkaline phosphatase (ALP)>1.5×ULN for the reference range;
c. total bilirubin>1×ULN for the reference range;
d. amylase or lipase>2×ULN for the reference range;
e. calcitonin≥20 ng/L;
f. estimated glomerular filtration rate (eGFR)<45 mL/min/1.73 m² by the 2021 CKD-EPI creatinine equation;
g. fasting plasma triglycerides≥500 mg/dL;
h. acute or chronic viral hepatitis (determined based on hepatitis B surface antigen [HbsAg] or presence of HCV RNA);
i. positive HIV antibody screen;
j. hemoglobin value <11 g/dL (men) or <10 g/dL (women);
k. white blood cells (WBC)<the lower limit of normal (LLN) for the reference range; or
l. platelets<LLN for the reference range.

### III. CT-388 Therapy

The pharmaceutical composition comprising CT-388 may be injected subcutaneously at an interval deemed appropriate by a physician, for example, every three days, every four days, every five days, every six days, every week, every two weeks, every three weeks, every four weeks, every eight weeks, every month, or every two months. The pharmaceutical composition comprising CT-388 may be injected subcutaneously by the patient (i.e., by self-administration).

In some embodiments, the pharmaceutical composition containing CT-388 is administered by injection at a body site (e.g., abdomen, upper arm, thighs, or hips) subcutaneously once weekly (QW), every two weeks (i.e., biweekly or Q2W), or monthly (QM). In some embodiments, the pharmaceutical composition comprising CT-388 is administered by subcutaneous injection at a certain interval (e.g., as described above) over a period of 4 to 28 weeks, or longer (e.g., 6, 12, 18, or 24 months, or longer). In some embodiments, the pharmaceutical composition is administered irrespective of meals.

In some embodiments, CT-388 is administered subcutaneously to an adult patient as described in the present disclosure (e.g., an overweight or obese adult patient who is otherwise healthy; an overweight or obese adult patient who has weight-related comorbidities but no diabetes; an overweight or obese adult patient who has T2DM but no comorbidities; or an overweight or obese adult patient who has T2DM and one or more comorbidities) at a flat dose of about 0.5 mg to about 30 mg, for example, about 1 mg to about 28 mg, about 2 mg to about 27 mg, about 3 mg to about 26 mg, about 4 mg to about 25 mg, about 5 mg to about 24 mg, about 5 mg to about 22 mg, about 8 mg to about 22 mg, about 8 mg to about 24 mg, about 5 mg to about 17 mg, about 2 mg to about 16 m, about 4 mg to about 16 mg, about 2 mg to about 20 mg, about 4 mg to about 20 mg, about 2 mg to about 24 mg, or about 4 mg to about 24 mg. In some embodiments, treatment with CT-388 is initiated on an up-titration schedule, i.e., starting treatment at a low dosing amount, and gradually increasing the dosing amount over a period of time to the higher or highest, tolerated dosing amount. In some embodiments, the dosing amount increases by an increment of 2 to 10 mg each time, e.g., by an increment of 2 mg, 3 mg, 4 mg, 5 mg, 6 mg, 7 mg, 8 mg, 9 mg, or 10 mg.

In some embodiments, a pharmaceutical composition comprising CT-388 is injected subcutaneously to an overweight or obese patient with or without T2DM (with or without comorbidities) at a QW, Q2W, or QM dose of about 0.5 mg.

In some embodiments, a pharmaceutical composition comprising CT-388 is injected subcutaneously to an overweight or obese patient with or without T2DM (with or without comorbidities) at a QW, Q2W, or QM dose of about 2 mg.

In some embodiments, a pharmaceutical composition comprising CT-388 is injected subcutaneously to an overweight or obese patient with or without T2DM (with or without comorbidities) at a QW, Q2W, or QM dose of about 4 mg.

In some embodiments, a pharmaceutical composition comprising CT-388 is injected subcutaneously to an overweight or obese patient with or without T2DM (with or without comorbidities) at a QW, Q2W, or QM dose of about 5 mg.

In some embodiments, a pharmaceutical composition comprising CT-388 is injected subcutaneously to an overweight or obese patient with or without T2DM (with or without comorbidities) at a QW, Q2W, or QM dose of about 6 mg.

In some embodiments, a pharmaceutical composition comprising CT-388 is injected subcutaneously to an overweight or obese patient with or without T2DM (with or without comorbidities) at a QW, Q2W, or QM dose of about 7.5 mg.

In some embodiments, a pharmaceutical composition comprising CT-388 is injected subcutaneously to an overweight or obese patient with or without T2DM (with or without comorbidities) at a QW, Q2W, or QM dose of about 8 mg.

In some embodiments, a pharmaceutical composition comprising CT-388 is injected subcutaneously to an overweight or obese patient with or without T2DM (with or without comorbidities) at a QW, Q2W, or QM dose of about 12 mg.

In some embodiments, a pharmaceutical composition comprising CT-388 is injected subcutaneously to an overweight or obese patient with or without T2DM (with or without comorbidities) at a QW, Q2W, or QM dose of about 16 mg.

In some embodiments, a pharmaceutical composition comprising CT-388 is injected subcutaneously to an overweight or obese patient with or without T2DM (with or without comorbidities) at a QW, Q2W, or QM dose of about 17 mg.

In some embodiments, a pharmaceutical composition comprising CT-388 is injected subcutaneously to an overweight or obese patient with or without T2DM (with or without comorbidities) at a QW, Q2W, or QM dose of about 20 mg.

In some embodiments, a pharmaceutical composition comprising CT-388 is injected subcutaneously to an overweight or obese patient with or without T2DM (with or without comorbidities) at a QW, Q2W, or QM dose of about 22 mg.

In some embodiments, a pharmaceutical composition comprising CT-388 is injected subcutaneously to an overweight or obese patient with or without T2DM (with or without comorbidities) at a QW, Q2W, or QM dose of about 24 mg.

In some embodiments, treatment with CT-388 is initiated on an up-titration schedule, i.e., starting treatment at a low dosing amount, and gradually increasing the dosing amount over a period of time to the higher or highest, tolerated dosing amount. In some embodiments, the titration occurs over a period of about 3, 4, 8, 12, 16, 20, 24, 28, or 32 weeks, or longer; in further embodiments, during the titration period, the pharmaceutical composition containing CT-388 is injected QW. In some embodiments, the titration starts with a dosing amount (starting dose) of 5 mg, and the dosing amount increases by an increment of 2 to 10 mg each time, e.g., by an increment of 2 mg, 3 mg, 4 mg, 5 mg, 6 mg, 7 mg, 8 mg, 9 mg, or 10 mg, finally reaching a dosing amount (maintenance or maximum dose) that is between 7.5 mg and 24 mg, for example, 22 mg. During the titration period, the patient may stay on a new dose for 1 to 10 weeks (e.g., 1, 2, 3, 4, 5, 6, 7, 8, 9, or 10 weeks) for acclimatization, before progressing to the next higher dose.

In some embodiments, the titration occurs over a period of 3 or more (e.g., 4) weeks according to the following regimen:
5 mg in week 1;
5 mg in week 2;
5 mg in week 3; and
7.5 mg in week 4.

In further embodiments, the patient treated is an overweight or obese patient without a weight-related comorbidity such as T2DM.

In some embodiments, the titration occurs over a period of 3 or more (e.g., 4) weeks according to the following regimen:
5 mg in week 1;
5 mg in week 2;
8 mg in week 3; and
12 mg in week 4.

In some embodiments, the titration occurs over a period of 3 or more (e.g., 4) weeks according to the following regimen:
5 mg in week 1;
8 mg in week 2;
12 mg in week 3; and
12 mg in week 4.

In some embodiments, the titration occurs over a period of 3 or more (e.g., 4) weeks according to the following regimen:
8 mg in week 1;
8 mg in week 2;
12 mg in week 3; and
16 mg in week 4.

In some embodiments, the titration occurs over a period of 3 or more (e.g., 4) weeks according to the following regimen:
8 mg in week 1;
12 mg in week 2;
16 mg in week 3; and
20 mg in week 4.

In some embodiments, the titration occurs over a period of 3 or more (e.g., 4) weeks according to the following regimen:
5 mg in week 1;
8 mg in week 2;
12 mg in week 3; and
16 mg in week 4.

In some embodiments, the titration occurs over a period of 3 or more (e.g., 4) weeks according to the following regimen:
5 mg in week 1;
5 mg in week 2;
5 mg in week 3; and
8 mg in week 4.

In further embodiments, the patient treated is an obese patient without a weight-related comorbidity such as T2DM.

In some embodiments, the titration occurs over a period of 8 or more (e.g., 9) weeks according to the following regimen:
5 mg in week 1;
5 mg in week 2;
8 mg in week 3;
8 mg in week 4;
12 mg in week 5;
12 mg in week 6;
17 mg in week 7;
17 mg in week 8; and
22 mg in week 9.

In further embodiments, the patient treated is obese with or without a weight-related comorbidity such as T2DM.

In some embodiments, the titration occurs over a period of 8 or more (e.g., 9) weeks according to the following regimen:
8 mg in week 1;
8 mg in week 2;
8 mg in week 3;
8 mg in week 4;
16 mg in week 5;
16 mg in week 6;
16 mg in week 7;
16 mg in week 8; and
24 mg in week 9.

In further embodiments, the patient treated is obese without a weight-related comorbidity such as T2DM.

In some embodiments, the titration occurs over a period of 8 or more (e.g., 9) weeks according to the following regimen:
8 mg in week 1;
8 mg in week 2;
12 mg in week 3;
12 mg in week 4;
16 mg in week 5;
16 mg in week 6;
20 mg in week 7;
20 mg in week 8; and
24 mg in week 9.

In further embodiments, the patient treated is obese without a weight-related comorbidity such as T2DM.

In some embodiments, the titration occurs over a period of 11 or more (e.g., 12) weeks according to the following regimen:
5 mg weekly in weeks 1-3; and
8 mg weekly in weeks 4-9.

In further embodiments, the patient treated is obese without a weight-related comorbidity such as T2DM.

In some embodiments, the titration occurs over a period of 23 or more (e.g., 25) weeks according to the following regimen:
2 mg in weeks 1-4;
4 mg in weeks 5-8,
8 mg in weeks 9-12,
12 mg in weeks 13-16,
16 mg in weeks 17-20,
20 mg in weeks 21-24, and
24 mg in week 25.

In further embodiments, the patient treated is with obesity or overweight with at least one weight-related comorbidity.

In some embodiments, the titration occurs over a period of 16 or more (e.g., 17) weeks according to the following regimen:
2 mg in weeks 1-4;
4 mg in weeks 5-8,
8 mg in weeks 9-12,
12 mg in weeks 13-16, and
16 mg in week 17.

In further embodiments, the patient treated is with obesity or overweight with at least one weight-related comorbidity.

In some embodiments, the titration occurs over a period of 12 or more (e.g., 13) weeks according to the following regimen:
2 mg in weeks 1-4;
4 mg in weeks 5-8,
8 mg in weeks 9-12, and
12 mg in weeks 13.

In further embodiments, the patient treated is with obesity or overweight with at least one weight-related comorbidity.

In some embodiments, the titration occurs over a period of four or more (e.g., five) weeks according to the following regimen:
4 mg in weeks 1-4, and
8 mg in week 5.

In further embodiments, the patient treated is with obesity or overweight with at least one weight-related comorbidity.

In some embodiments, the titration occurs over a period of 23 or more (e.g., 25) weeks according to the following regimen:
2 mg in weeks 1-4;
4 mg in weeks 5-8,
8 mg in weeks 9-12,
12 mg in weeks 13-16,
16 mg in weeks 17-20,
20 mg in weeks 21-24, and
24 mg in week 25.

In further embodiments, the patient treated is overweight or obese with type 2 diabetes mellitus.

In some embodiments, the titration occurs over a period of 16 or more (e.g., 17) weeks according to the following regimen:
2 mg in weeks 1-4;
4 mg in weeks 5-8,
8 mg in weeks 9-12,
12 mg in weeks 13-16, and
16 mg in week 17.

In further embodiments, the patient treated is overweight or obese with type 2 diabetes mellitus.

In some embodiments, the titration occurs over a period of four or more (e.g., five) weeks according to the following regimen:
4 mg in weeks 1-4, and
8 mg in week 5.

In further embodiments, the patient treated is overweight or obese with type 2 diabetes mellitus.

In some embodiments, the titration occurs according to the following regimen:
2 mg in weeks 1-12, and
4 mg in week 13.

In further embodiments, the patient treated is overweight or obese with type 2 diabetes mellitus.

In some embodiments, the titration occurs according to the following regimen:
4 mg in weeks 1-12, and
8 mg in week 13.

In further embodiments, the patient treated is overweight or obese with type 2 diabetes mellitus.

In some embodiments, the titration occurs according to the following regimen:
4 mg in weeks 1-4,
8 mg in weeks 5-8,
12 mg in weeks 9-12, and
16 mg in week 13.

In further embodiments, the patient treated is overweight or obese with type 2 diabetes mellitus.

In some embodiments, the titration occurs according to the following regimen:
2 mg in weeks 1-4,
4 mg in weeks 5-8,
8 mg in weeks 9-12,
12 mg in weeks 13-16,
16 mg in weeks 17-20,
20 mg in weeks 21-24, and
24 mg in week 25.

In further embodiments, the patient treated is overweight or obese with type 2 diabetes mellitus.

When titrating dose upwards, if a dose is not tolerated at any stage, the dosing may be reverted to the previous tolerated dose for the next dose (i.e., down-titration is allowed).

In some embodiments, the patient may then attempt up-titration at the subsequent scheduled dosing time.

In some embodiments, a pharmaceutical composition comprising CT-388 is administered to an overweight or obese patient with or without weight-related comorbidities (e.g., T2DM) as an adjunct to diet and/or exercise therapy.

In some embodiments, a pharmaceutical composition comprising CT-388 is administered to an overweight or obese patient with T2DM (with or without comorbidities) as an adjunct to another anti-diabetic treatment, e.g., treatment with metformin.

In some embodiments, a pharmaceutical composition comprising CT-388 administered to an overweight or obese patient with or without T2DM (with or without comorbidities) in lieu of bariatric surgery.

In some embodiments, CT-388 may be administered in combination with one or more of additional therapeutic agents. Representative additional therapeutic agents include, but are not limited to, anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, anti-oxidants, therapeutic agents for osteoporosis, vitamins, antidementia drugs, erectile dysfunction drugs, therapeutic drugs for urinary frequency or urinary incontinence, therapeutic agents for NAFLD, and therapeutic agents for dysuria. In some embodiments, CT-388 is administered in combination with a therapeutic agent that preserves muscle mass.

### IV. Treatment Outcomes

In some embodiments, patients treated with CT-388 experience weight loss. For example, patients may experience 3 or more (e.g., 3.7 or more, 4 or more, 5 or more, 6.0 or more, 7.0 or more, or 7.4 or more) kg of placebo-adjusted weight loss. In some embodiments, obese patients experience 7.0 kg or more (e.g., 7.7 kg or more) of weight loss. Patients may also experience placebo-adjusted weight loss of 5% or more (e.g., 9.3% or more, 9.6% or more, 10% or more, 10.2% or more, 10.4% or more, 10.9% or more, 11.5% or more, 11.8% or more, 12.2% or more, 12.4% or more, 12.6% or more, 13% or more, 13.10% or more, 15% or more, 18.3% or more, 18.8% or more, 18.9% or more, 20% or more, or 20.7 or more) of their initial body weight.

In some embodiments, patients treated with CT-388 experience a decrease in waist circumference. For example, patients may experience a decrease in waist circumference of 7 or more (e.g., 9 or more, 10 or more, or 13.4 or more) cm.

In some embodiments, CT-388 treatment lowers fasting glucose, fasting insulin, fasting C-peptide, and/or HOMA-IR in a patient. For example, fasting glucose may be decreased by about 4 or more (e.g., 5 or more, 6 or more, 7 or more, 8 or more, 9 or more, 10 or more, 11 or more, 12 or more, 13 or more, 14 or more, or 15 or more) mg/dL. Fasting insulin may be decreased by 1 or more (e.g., 2 or more, 3 or more, or 4 or more) mIU/L. Fasting C-Peptide may be decreased by 0.2 or more (e.g., 0.3 or more, 0.4 or more, 0.5 or more, 0.6 or more, 0.7 or more, 0.8 or more, or 0.9 or more) ng/mL. HOM-IR may decrease by 0.4 or more (e.g., 0.46 or more, 0.5 or more, 0.6 or more, 0.7 or more, 0.8 or more, 0.9 or more, or 1 or more).

Treatment may also decrease the placebo-adjusted AUC_{0-120 min} for glucose, insulin, or C-peptide in a patient. In some embodiments, the AUC_{0-120 min} for glucose is decreased by 60 or more (e.g., 66 or more, 70 or more, 80 or more, 88 or more, 90 or more, or 97 or more) mg*h/dL. In some embodiments, the AUC_{0-120 min} for insulin is decreased by 5 or more (e.g., 10 or more, 20 or more, 30 or more, 40 or more, 50 or more, 60 or more, 70 or more, 71 or more, 80 or more, 90 or more, 100 or more, 110 or more, 120 or more, or 127 or more) mIU*h/L. C-peptide may be decreased by 0.6 or more (e.g., 0.7 or more, 1.0 or more, 2.0 or more, 3.0 or more, 4.0 or more, 5.0 or more, or 5.1 or more) ng*h/mL.

In some embodiments, treatment decreases peak glucose excursion (Cₘₐₓ) by 10% or more (e.g., 15% or more, 16% or more, 20% or more, 25% or more, 30% or more, or 31% or more) compared to placebo. In some embodiments, treatment decreases Cₘₐₓ of insulin by 20% or more (e.g., 35% or more, 40% or more, 45% or more, 50% or more, or 55% or more). In some embodiments, C-peptide levels are decreased by 15% or more (e.g., 17% or more, 20% or more, 25% or more, 30% or more, 35% or more, or 39% or more).

Treatment may decrease HOMA-IR to about 1 to about 3 (e.g., to 1.15, to 1.75, or to 2.37). In some embodiments, HbA1c in a patient treated with CT-388 is reduced by −0.03 or −0.04.

Treatment may normalize glucose metabolism in a patient, or improve insulin sensitivity in a patient. Pre-diabetic patients who undergo CT-388 treatment may become normoglycemic.

In some embodiments, the CT-388 therapy herein results in improvement in one or more of weight and metabolic (e.g., glycemic) parameters, including, without limitation, body weight, fasting glucose, fasting insulin, HOMA-IR, waist and hip circumference, fructosamine, HbA1c, fasting lipid profile, fasting free fatty acids, ketone bodies, adiponectin, MRI-PDFF, and biomarkers (ALT, PRO-C3, CK18, and FIB-4).

### V. Articles of Manufacture and Formulations

The present disclosure provides an article of manufacture (e.g., a kit) for use in the present therapy. The article of manufacture may contain one or more doses of CT-388. The CT-388 dose(s) may be housed in a pre-filled syringe or injector, optionally a single-use syringe or injector. The article of manufacture may also include use instructions.

In some embodiments, CT-388 is provided in a pharmaceutical composition. The pharmaceutical composition may comprise CT-388 at a concentration of 20 mg/mL, and may also comprise a sodium phosphate buffer or mannitol, optionally at pH 7.0. In some embodiments, the pharmaceutical composition comprises CT-388 at a concentration of 20 mg/mL, sodium phosphate buffer, and mannitol at pH 7.0.

The pharmaceutical composition may comprise the components set out in Table 2.

TABLE 2
CT-388 Hydrochloride Salt Drug Product Composition

| | Amount per Vial | | Quality |
|---|---|---|---|
| Component | (mg/mL) | Function | Standard |
| CT-388 | 20 | Active Ingredient | In-house |
| Sodium phosphate monobasic | 1.38 | Buffer | USP/NF, BP, |
| monohydrate | | | Ph. Eur., JP |
| Trehalose dihydrate | 25 | Tonicity modifier/ | USP/NF, |
| | | Bulking agent | Ph. Eur., JP |
| Mannitol | 40 | Tonicity modifier/ | USP/NF, BP, |
| | | Bulking agent | Ph. Eur. |
| 10% Polysorbate 20 solution | 1 | Surfactant | USP/NF, Ph. |
| | | | Eur., JP |
| 1N NaOH | q.s. to pH 6.5 ± 0.2 @ | pH adjustment | USP/NF, BP, |
| | 25 ± 1° C. | | Ph. Eur., JP |
| Water for Injection | q.s. | Solvent | USP/NF |

Abbreviations: BP: British Pharmacopeia; NF: National Formulary; Ph. Eur.: European Pharmacopeia; JP: Japanese Pharmacopeia: q,s: Quantum satis, water added in the amount needed to fill 1.0 mL; USP: United States Pharmacopeia

In some embodiments, the pharmaceutical composition is provided in a vial (e.g., a 5 mL glass vial). The vial may be a 20 mm glass vial with a single vent lyo stopper closed with a 20 mm flip-off TruEdge matte white seal. In some embodiments, each vial comprises 1.0 mL of the pharmaceutical composition. In some embodiments, each vial comprises 20 mg of CT-388.

### VI. Exemplary Embodiments

Non-limiting, exemplary embodiments are provided below to further illustrate the present disclosure.

1. A compound, or a pharmaceutically acceptable salt or ester thereof, for use in
managing weight,
improving glycemic control, and/or
increasing insulin sensitivity
in an overweight or obese adult patient in need thereof, wherein the compound is of Formula I or Formula II:

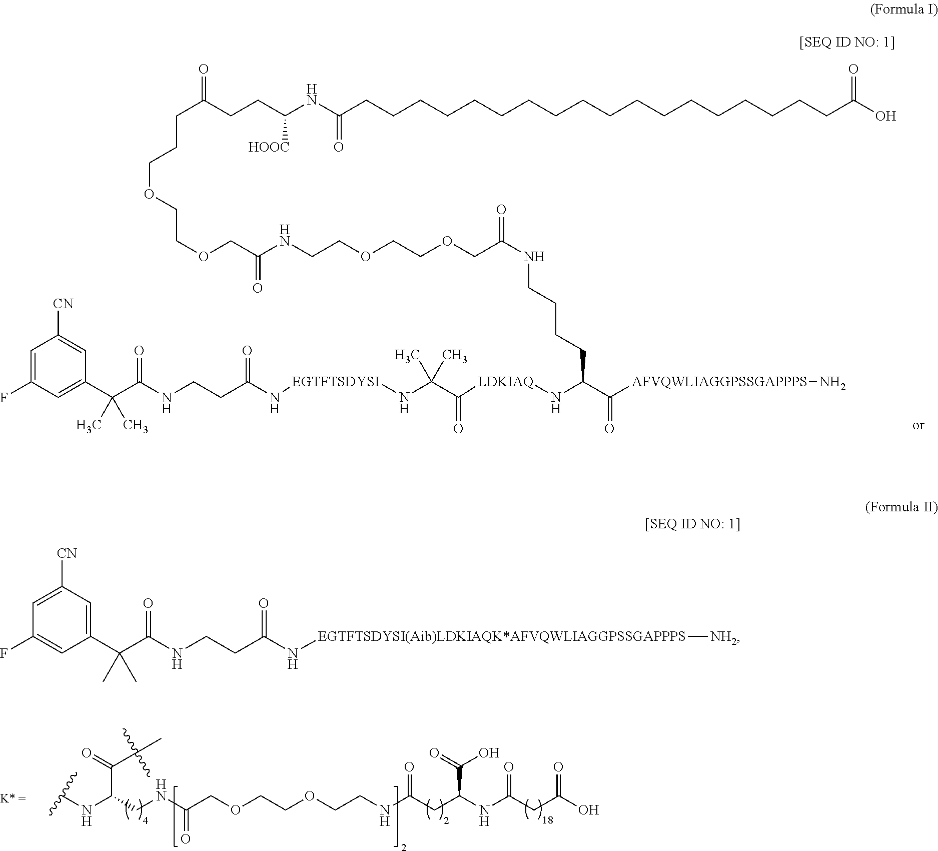

wherein the compound is to be administered subcutaneously at a body weight-independent dose of about 2.0 mg to about 25 mg.
   2. The compound, or pharmaceutically acceptable salt thereof, for use of embodiment 1, wherein the patient has a BMI of ≥25 kg/m², ≥27 kg/m², ≥30 kg/m², ≥35 kg/m², or ≥40 kg/m².
   3. The compound, or pharmaceutically acceptable salt thereof, for use of embodiment 1, wherein the patient is obese.
   4. The compound, or pharmaceutically acceptable salt thereof, for use of embodiment 3, wherein the patient does not have type 2 diabetes mellitus, or is prediabetic.
   5. The compound, or pharmaceutically acceptable salt thereof, for use of embodiment 3, wherein the patient has type 2 diabetes mellitus.
   6. The compound, or pharmaceutically acceptable salt thereof, for use of any one of embodiments 1-5, wherein the patient has one or more weight-related comorbidities, optionally prediabetes, hypertension, or dyslipidemia.
   7. The compound, or pharmaceutically acceptable salt thereof, for use of any one of embodiments 1-6, wherein the administering step is repeated once every week, once every two weeks, once every four weeks, or once every month.
   8. The compound, or pharmaceutically acceptable salt thereof, for use of any one of the preceding embodiments, wherein the dose is about 5, 6, 7.5, 8, 12, 16, 17, 22, or 24 mg.
   9. The compound, or pharmaceutically acceptable salt thereof, for use of any one of the preceding embodiments, wherein the dose is up-titrated over a period of 3 weeks to 30 weeks.
   10. The compound, or pharmaceutically acceptable salt thereof, for use of embodiment 9, wherein the starting dose for the up-titration is about 5 mg or about 8 mg.
   11. The compound, or pharmaceutically acceptable salt thereof, for use of embodiment 10, wherein the maximum dose for the up-titration is about 22 or 24 mg.
   12. The compound, or pharmaceutically acceptable salt thereof, for use of any one of the preceding embodiments, wherein the patient receives an additional therapy for overweight or obesity, optionally wherein the additional therapy is diet therapy or exercise therapy.
   13. The compound, or pharmaceutically acceptable salt thereof, for use of any one of the preceding embodiments, wherein administration of the compound results in reduction of visceral adiposity and/or HbA1c level in the patient.
   14. An article of manufacture comprising the compound, or pharmaceutically acceptable salt thereof, for use of any one of embodiments 1-13, wherein the article of manufacture comprises one or more units of said dose.
   15. The article of manufacture of embodiment 14, wherein the article of manufacture comprises a syringe or an injector, optionally a single-use syringe or injector.
   16. A compound, or a pharmaceutically acceptable salt or ester thereof, for use in treating obesity in an adult patient in need thereof, wherein the compound is of Formula I or Formula II:

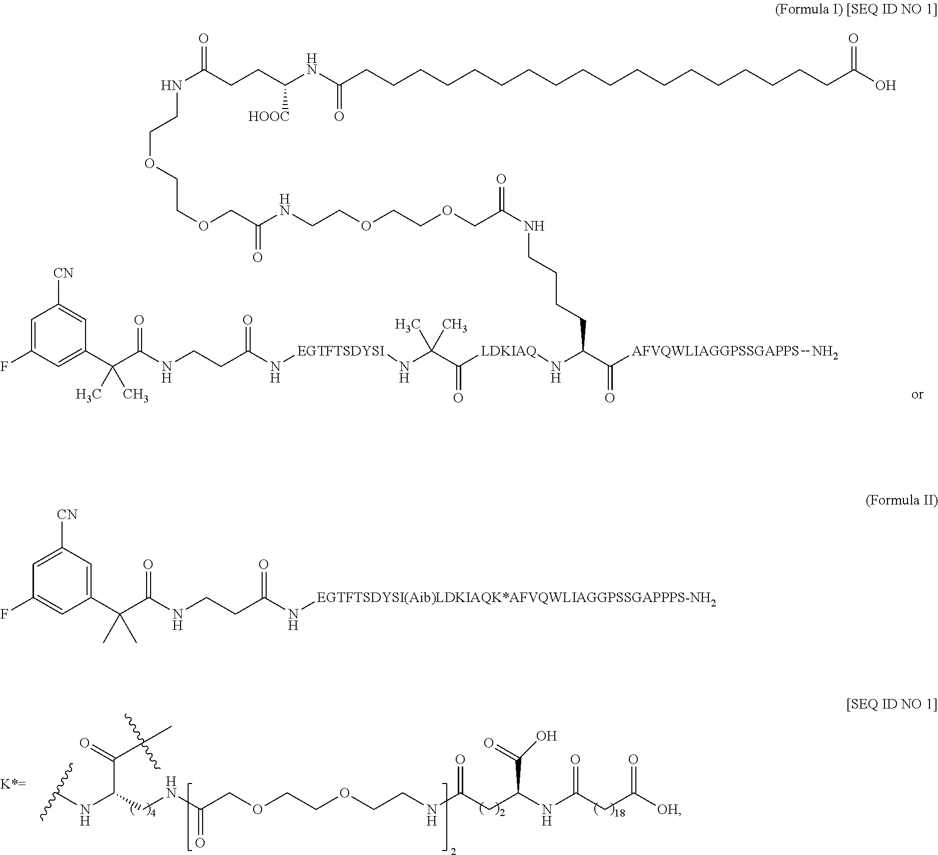

wherein the compound is to be administered subcutaneously at a body weight-independent dose of about 2.0 mg to about 25 mg.
   17. A compound, or a pharmaceutically acceptable salt or ester thereof, for use in treating type 2 diabetes mellitus in an adult patient in need thereof, wherein the compound is of Formula I or Formula II:

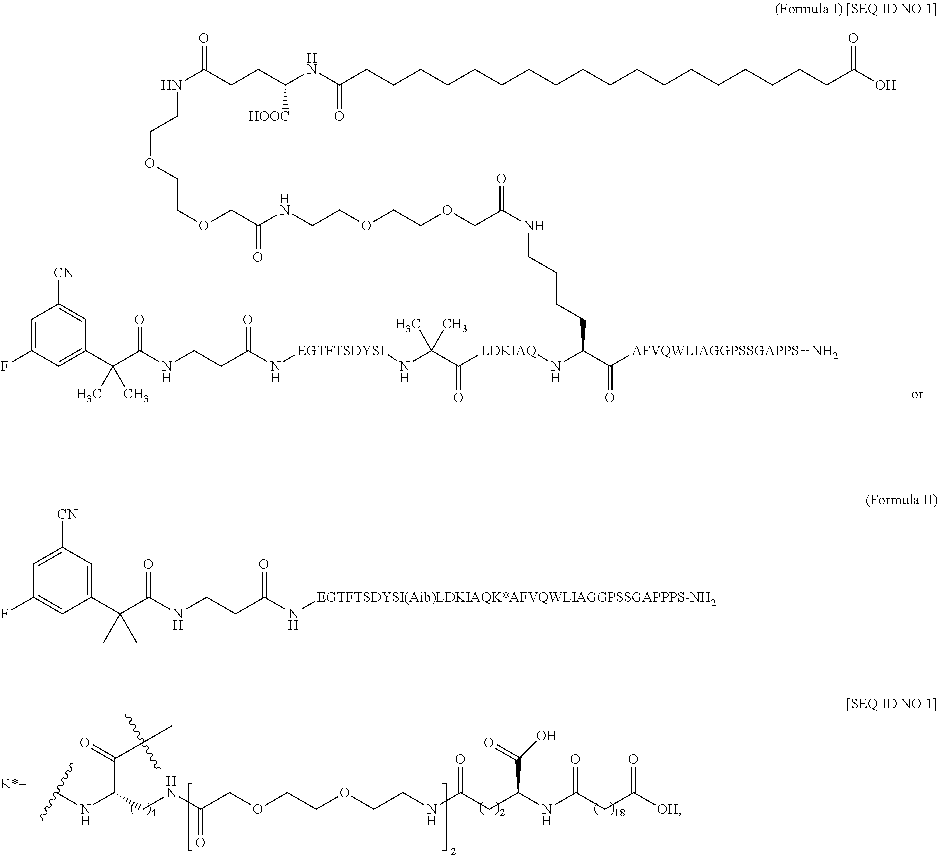

wherein the compound is to be administered subcutaneously at a body weight-independent dose of about 2.0 mg to about 25 mg.
   18. The compound, or pharmaceutically acceptable salt or ester thereof, for use of embodiment 17, wherein the patient is overweight or obese.
   19. A compound, or a pharmaceutically acceptable salt or ester thereof, for use in treating obesity or type 2 diabetes mellitus in an adult patient in need thereof, wherein the compound is of Formula I or Formula II:

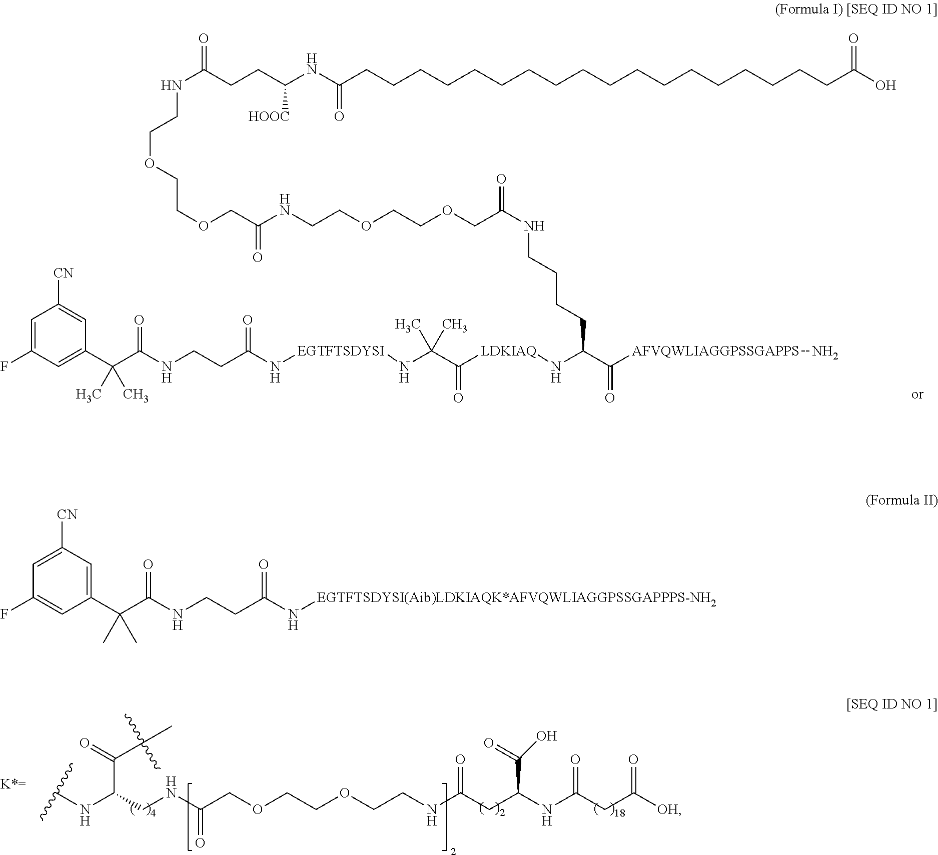

wherein the compound is to be administered subcutaneously at a body weight-independent dose of about 2.0 mg to about 25 mg.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Any compound disclosed herein can be used in any of the treatment method here, wherein the individual to be treated is as defined anywhere herein. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: A Phase 1 Clinical Trial Protocol

This Example outlines the protocol for a Phase 1 randomized, double-blind, placebo-controlled study to evaluate the safety, tolerability, pharmacokinetics, and pharmacodynamics of CT-388 in otherwise healthy overweight and obese adult participants and in obese patients with type 2 diabetes mellitus.

For once weekly dosing, CT-388 is supplied as a 1-mL or 2-mL lyophilized powder or sterile solution at a concentration of 20 mg/mL, or placebo, as sterile solutions for subcutaneous (SC) injection.

### A. Objectives

### Single-Ascending Dose (SAD) Once Weekly (QW)

The primary objective of this study is to determine the safety and tolerability of CT-388 in otherwise healthy overweight and obese adult participants when administered as a single dose of 0.5, 2.0, 5.0, 6.0, or 7.5 mg.

The secondary objectives are:
To evaluate the pharmacokinetics of single-ascending doses of CT-388 in otherwise healthy overweight and obese adult participants; and
To evaluate the pharmacodynamic (PD) effect of a single dose of CT-388 on body weight and glucose homeostasis in both the fasted state and in the setting of a mixed meal tolerance test (MMTT) challenge in otherwise healthy overweight and obese adult participants.

### Multiple-Ascending Dose (MAD) QW

The primary objective of this study is to determine the safety and tolerability of CT-388 in otherwise healthy overweight and obese adult participants when administered once weekly over 4 consecutive weeks with titrated dosing.

The secondary objectives of this study are:
To evaluate the pharmacokinetics of CT-388 when titrated across 4 consecutive weekly doses; and
To evaluate the PD effect of 4 consecutive weekly titrated doses of CT-388 on body weight and glucose homeostasis in both the fasted state and in the setting of an oral glucose tolerance test (OGTT) in otherwise healthy overweight and obese participants.

### Multiple Dose (MD) QW

The primary objective of this study is to determine the safety and tolerability of CT-388 in participants with and without type 2 diabetes mellitus (T2DM) when administered once weekly over 12 consecutive weeks for Cohorts 11 and 14 with titrated dosing (and additionally, up to 24 weeks for Cohorts 12 and 13).

The secondary objectives of this study are:
To evaluate the pharmacokinetics of CT-388 across 12 consecutive weekly doses for Cohorts 11 and 14 (and additionally, up to 24 weeks for Cohorts 12 and 13); and
To evaluate the PD effect of CT-388 across 12 consecutive weekly doses for Cohorts 11 and 14 (and additionally up to 24 weeks for Cohorts 12 and 13) on body weight and glucose homeostasis in both the fasted state and in the setting of an OGTT in participants with or without T2DM.

The exploratory objectives of this study are:
To evaluate the PD effect of CT-388 when titrated across 12 consecutive weekly doses and additionally up to 24 weeks in participants without T2DM (Cohort 12) or with T2DM (Cohort 13) on MRI-PDFF (proton density fat fraction) and adiposity; and
To evaluate the PD effect of CT-388 when titrated across 12 consecutive weekly doses in Cohorts 11-14 on biomarkers (e.g., lipid profile, adiponectin, alanine aminotransferase (ALT), Pro-C3, CK18, FIB-4), and additionally up to 24 weeks in participants without T2DM (Cohort 12) or with T2DM (Cohort 13).

### B. Endpoints

Safety and tolerability will be assessed by monitoring adverse events (AE) including local injection-site reactions, measuring vital signs and ECGs, and performing clinical laboratory blood and urine analyses.

### C. Pharmacodynamics

The PD evaluation includes changes in body weight, fasting glucose, fasting insulin, HOMA-IR, waist and hip circumference, fructosamine, HbA1c, fasting lipid profile, fasting free fatty acids, ketone bodies, adiponectin, MRI-PDFF, biomarkers (ALT, PRO-C3, CK18, and FIB-4), as well as AUC, baseline-adjusted AUC, Cmax, and Tmax from baseline to a pre-determined day.

### D. Main Criteria for Inclusion

Cohorts 1-6 will include overweight or obese otherwise healthy adult male or female participants aged 18-65 years (inclusive) with a body mass index (BMI)≥25.0 kg/m² (inclusive, at Screening).

Cohorts 7-12 and 14 will include only obese otherwise healthy adult male or female participants aged 18-65 years (inclusive) with a BMI≥30.0 kg/m² (inclusive, at Screening). Abnormalities in blood pressure (i.e., treated hypertension), serum lipids (i.e., treated hyperlipidemia), and serum glucose values consistent with a diagnosis of prediabetes are acceptable.

Cohort 13 will include adult male or female participants aged 18-65 years (inclusive) with a BMI≥30.0 kg/m² (inclusive, at Screening) and have a diagnosis of T2DM for ≥6 months that is managed with diet and exercise alone, or treated with a stable dose of metformin monotherapy for at least 3 months prior to Screening Visit and with an HbA1c between 7.0% and 10.0%. Other abnormalities of serum glucose, serum lipids, urinary glucose and urinary protein consistent with a diagnosis of T2DM are acceptable.

### E. Inclusion Criteria

Inclusion criteria for participants of all cohorts include the following:
1) Males or females, between 18 and 65 years of age, inclusive, at time of consent.
2) Female participants must not be pregnant, or breast feeding.
3) BMI≥25.0 kg/m², inclusive at Screening and Admission (Day −2/Day −1) for Cohorts 1-6, and BMI≥30.0 kg/m², inclusive at Screening and Admission (Day 2/Day 1) for Cohorts 7-14.
4) Stable body weight (defined as ≤4.0 kg gain or loss) in the 2 months prior to Screening as per verbal report by participant and for the period between Screening and Admission (Day −2/Day −1) as per measured weight.
5) No clinically significant symptoms, signs, or active manifestations of stable conditions in the opinion of the Principal Investigator (PI) or delegate from medical history, physical exam, vital signs, and laboratory findings at Screening and Admission (Day −2/Day −1).

### F. Exclusion Criteria

Exclusionary criteria for participants of all cohorts include the following:
1) Clinically significant history or active clinical manifestation(s) of any significant metabolic, allergic, dermatological, hepatic, renal, hematological, pulmonary, cardiovascular, gastrointestinal, neurological, or psychiatric disorder (as determined by the PI or delegate). However, based on the judgement and discretion of the Investigator or delegate the following exceptions may be considered:
   a) Participants with non-severe gastroesophageal reflux if stable on therapy with a proton pump inhibitor for >3 months prior to Screening may be allowed.
   b) Participants with stable asthma (i.e., no exacerbations or use of asthma relievers within 3 months prior to Screening) and anticipated to be stably managed for the duration of the study may be allowed.
   c) Participants with stable depression being treated with stable doses and regimen of selective serotonin reuptake inhibitors (SSRIs) or serotonin norepinephrine reuptake inhibitors (SNRIs) for more than 12 months prior to screening may be allowed; however, a history of depression requiring inpatient admission/ECT/past treatment with antipsychotics and/or requiring ongoing psychotherapy and/or on treatment with any central nervous system stimulants (e.g., Ritalin-SR® for ADHD) is exclusionary.
   d) Participants may be on medications for the control of hypertension (e.g., ACE-inhibitors) or hyperlipidemia (e.g., statins/fibrates) provided that their blood pressure and lipid parameters are stably controlled, and must be on a dose and regimen that has been stable for more than 3 months prior to the Screening Visit, and that the medication/dose/regimen is not anticipated to change during the course of the study.
2) Current diagnosis of any type of diabetes (as defined by the 2022 American Diabetes Association Standards of Medical Care in Diabetes (Clin Diabetes (2022) 40(1):10-38), except for Cohort 13 where diagnosis of T2DM is required)
   a) Participants with prediabetes (as defined by the 2022 American Diabetes Association Standards of Medical Care in Diabetes (Clin Diabetes (2022) 40(1):10-38) may be allowed, but only if they are controlled and maintained on diet/exercise alone (i.e., no pharmacotherapy of any type, including no metformin, nor any other type of over-the-counter/herbal/nonprescription/supplement treatments are allowed).
3) History of bariatric surgery (of any type) or gastric banding (including LAP-BAND), history of any gastrointestinal surgery that may induce malabsorption (e.g., bowel resection), or any GI motility disorders, gastroparesis, delayed gastric emptying, inflammatory bowel disease, pancreatitis, malabsorption syndromes, chronic diarrhea, chronic constipation, clinically significant irritable bowel syndrome.
4) Semi-supine blood pressure systolic>160 mm Hg and/or diastolic>95 mm Hg at Screening, Admission (Day 2/Day 1), and Day 1 (predose).
5) An active or untreated malignancy or have been in remission from a clinically significant malignancy (other than basal or squamous cell skin cancer, in situ carcinomas of the cervix, or in situ prostate cancer) for <5 years prior to screening. Personal or family history (first-degree relative) of medullary thyroid carcinoma (MTC), or a genetic condition that predisposes to MTC (i.e., thyroid C-cell hyperplasia, or multiple endocrine neoplasia type 2A or type 2B)
6) History of atopy (severe or multiple allergic manifestations) or clinically significant multiple or severe drug allergies, or intolerance to topical corticosteroids, or severe posttreatment hypersensitivity reactions; known allergy to any component of the study drug, GLP-1 analogues, or related compounds
7) Any of the following laboratory abnormalities at Screening Visit (some exceptions to this criterion would apply to Cohort 13 where diagnosis of T2DM is required; see below):
   a) Aspartate transaminase [AST], alanine aminotransferase [ALT], alkaline phosphatase [ALP], gamma glutamyl transpeptidase [GGT]>2× the upper limit of normal (ULN) or
   b) Total bilirubin>ULN
   c) Amylase and Lipase>ULN
   d) Fasting triglycerides≥250 mg/dL (2.83 mmol/L)
   e) Estimated glomerular filtration rate (eGFR)<60 mL/min/1.73 m² as calculated by the central laboratory using the Chronic Kidney Disease Epidemiology Collaboration (CKD-EPI) formula at Screening.
   f) HbA1c level≥6.5%
   g) Fasting glucose≥126 mg/dL (6.9 mmol/L)
   h) Hemoglobin<11 g/dL (males) or <10 g/dL (females)
8) Current use or use within 3 months prior to Screening of any prescribed or non-prescribed drugs that are known to interfere with gut motility including but not limited to chronic opioids, anticholinergics, and antispasmodics.
9) Use of hormone replacement therapy (other than oral contraceptive products/IUDs/IUSs) 60 days prior to Admission (Day −2/Day −1).

For eligibility to enroll into Cohort 13 (multiple doses for 24 weeks in obese participants with T2DM), all the above inclusion and exclusion criteria should be met, including meeting the following additional criteria:
1) Diagnosis of T2DM as defined by the 2022 American Diabetes Association Standards of Medical Care in Diabetes (Clin Diabetes (2022) 40(1):10-38) for at least 6 months prior to Screening Visit,
2) T2DM should be managed with diet and/or exercise alone or treated with a stable dose of metformin monotherapy (either immediate release or extended release ≥500 mg/day and not more than the locally approved dose) for at least 3 months prior to the Screening Visit.
3) BMI≥30.0 kg/m²,
4) HbA1c between 7.0% and 10.0%, inclusive,
5) Alanine transaminase, aspartate transaminase, alkaline phosphatase, gamma glutamyl transpeptidase) up to 3× the upper limits of the normal (ULN) range and total bilirubin up to 1.5×ULN at Screening may be allowed; other abnormalities in serum (e.g., glucose, lipids) and urine (e.g., glucose, protein) consistent with a diagnosis of T2DM are acceptable,
6) Fasting serum triglyceride levels up to 500 mg/dL (5.6 mmol/L) at Screening may be allowed,
7) eGFR should be >45 mL/min/1.73 m² as calculated by the central laboratory using the CKD-EPI formula at Screening,
8) Fasting blood glucose should be <270 mg/dL (14.8 mmol/L) at Screening.

### G. Test Product(s), Dose, and Mode of Administration

Participants will receive one of the following treatments:

### SAD QW—Cohorts 1-5

Cohort 1: One dose containing 0.5 mg of CT-388 or matched placebo administered subcutaneously in the abdomen
Cohort 2: One dose containing 2.0 mg of CT-388 or matched placebo administered subcutaneously in the abdomen
Cohort 3: One dose containing 5.0 mg of CT-388 or matched placebo administered subcutaneously in the abdomen
Cohort 4: One dose containing 7.5 mg of CT-388 or matched placebo administered subcutaneously in the abdomen
Cohort 5: One dose containing 6.0 mg of CT-388 or matched placebo administered subcutaneously in the abdomen

### MAD QW—Cohorts 6-10

Cohort 6: One weekly dose titrated across 4 consecutive weeks with CT-388 doses of . . .
   5.0 mg at Day 1
   5.0 mg at Day 8
   5.0 mg at Day 15
   7.5 mg at Day 22
. . . or matched placebo administered subcutaneously in the abdomen
Cohort 7: One weekly dose titrated across 4 consecutive weeks with CT-388 doses of . . .
   5.0 mg at Day 1
   5.0 mg at Day 8
   8.0 mg at Day 15
   12.0 mg at Day 22
. . . or matched placebo administered subcutaneously in the abdomen
Cohort 8: One weekly dose titrated across 4 consecutive weeks with CT-388 doses of . . .
   5.0 mg at Day 1
   8.0 mg at Day 8
   12.0 mg at Day 15
   12.0 mg at Day 22
. . . or matched placebo administered subcutaneously in the abdomen (the third dose in Cohort 8 can be lowered to 8.0 mg based on tolerability data from Cohort 7)
Cohort 9: One weekly dose titrated across 4 consecutive weeks with CT-388 doses of . . .
   8.0 mg at Day 1
   8.0 mg at Day 8
   12.0 mg at Day 15
   16.0 mg at Day 22
. . . or matched placebo administered subcutaneously in the abdomen
Cohort 10: the following two dosing options are proposed in Table 3 below:

TABLE 3
Cohort 10 Options

| Option 1 | Option 2 |
|---|---|
| 8.0 mg at Day 1 | 5.0 mg at Day 1 |
| 12.0 mg at Day 8 | 8.0 mg at Day 8 |
| 16.0 mg at Day 15 | 12.0 mg at Day 15 |
| 20.0 mg at Day 22 | 16.0 mg at Day 22 |

Note:
The 4^{th} dose could be lowered to 16.0 mg based on tolerability data from Cohort 9

### MD QW—Cohorts 11-14

The primary aim for these cohorts is to enable a longer titration regimen to better delineate the tolerability profile to allow each participant to get to their individual maximal tolerated dose. In Cohorts 12 and 13, participants would also have an opportunity to further continue on such a dose for an additional 12 weeks (i.e., 24 weeks of dosing in total) to better assess safety and tolerability of the study product over an extended period.

In general, when titrating dose upwards, if a dose is not tolerated at any stage, revert to the previous tolerated dose for the next dose (i.e., down-titration is allowed), then attempt up-titration at the subsequent scheduled dosing time/week.

For all MD QW cohorts, the following should be performed to enable smooth up-titration of doses:
Flexible and an individual-based titration schedule tailored to the participant's specific tolerability level,
Temporarily withholding the study drug, and/or
Managing relevant AEs by reinforcing conservative measures (i.e., eating smaller-sized meal portions, less frequent/skipping meals), as well as with appropriate concomitant medications as clinically indicated.

The cohorts in the MD QW study are as follows.
Cohort 11: Once weekly dose administered SC in the abdomen across 12 consecutive weeks with CT-388 or matched placebo doses of 5.0 mg administered for the first 3 weeks and then titrated to 8.0 mg for the duration of the treatment period. If a participant(s) experiences moderate to severe tolerability issues after the third dose of 5 mg, the same 5 mg dose may be continued for up to an additional 2 weeks, i.e., up-titration to 8 mg may be attempted after the fourth or fifth 5 mg dose. If participant(s) still unable to tolerate 8 mg by Day 36, then that participant(s) may continue at 5 mg for the remaining duration of the treatment period.
Cohorts 12 and 13: Once weekly dose administered SC in the abdomen titrated across 12 consecutive weeks with CT-388 or matched placebo doses of:
   5.0 mg at Day 1 and Day 8
   8.0 mg at Day 15 and Day 22
   12.0 mg (or 8.0 mg based on tolerability) at Day 29 and Day 36
   17.0 mg (or 12.0 mg based on tolerability) at Day 43 and Day 50
   22.0 mg (or 12.0 mg or 17.0 mg based on tolerability) at Day 57.
   If at 22 mg on Day 57, continue that dose for the duration of the treatment period.
   If at 12 mg or 17 mg on Day 57, titration to the corresponding next higher dose level may be attempted on a weekly basis based on the individual's tolerability (e.g., if on Day 57, 12 mg dose was administered and tolerated, on Day 64, dose of 17 mg may be attempted).
   If not able to tolerate, continue at the previously tolerated dose level until the following week (e.g., to Day 71), at which point another attempt can be made to the next higher dose level.
   Attempts to the next higher dose level can continue to be made until Day 78. The dose level achieved at Day 78 should be maintained for an additional 12 weeks of weekly dosing in the extension period, resulting in a cumulative of 24 weeks of total doses for participants in Cohorts 12 and 13. At any time during the extension period, if the maintenance dose is not tolerated, then that dose may be either lowered by one corresponding dose level (e.g., if at 22 mg, decrease to 17 mg; if at 17 mg decrease to 12 mg), and/or the dosing may be withheld for up to 2 weeks. Following this temporary dose adjustment/withholding period, attempt to titrate back up to the previous (higher) dose level should be made. If that still results in poor toleration or if the Investigator believes participant may discontinue study due to poor toleration, the previously tolerated dose (as long as it is at least 12 mg) may be continued for the remainder of the extension period.
Dosing in Cohort 14 may follow one of these two dosing options shown in Table 4 below.

TABLE 4
Cohort 14 Options

| OPTION 1 | OPTION 2 |
|---|---|
| Minimum dose to tolerate is 16 mg | Minimum dose to tolerate is 12 mg |
| Once weekly dose administered SC in the | Once weekly dose administered |
| abdomen titrated across 12 consecutive | SC in the abdomen titrated across |
| weeks with CT-388 or matched placebo | 12 consecutive weeks with CT-388 or |
| doses of: | matched placebo doses of: |
| 8.0 mg at Day 1, Day 8, Day 15, | 8.0 mg at Day 1 and Day 8 |
| and Day 22 | 12.0 mg at Day 15 and Day 22 |
| 16.0 mg at Day 29, Day 36, Day 43, | 16.0 mg (or 12.0 mg based on |
| and Day 50 | tolerability) at Day 29 and Day |
| 12.0 mg may be provided on Day 36 | 36 |
| and Day 43 if 16 mg is not tolerated | 20.0 mg (or 12.0 mg or 16 mg |
| on Day 29 | based on tolerability) at Day |
| Attempts must be made to get to 16 | 43 and Day 50 |
| mg by Day 50; however, if an | 24.0 mg (or 12.0 mg or 16.0 mg or |
| individual participant is | 20 mg based on tolerability) at |
| experiencing tolerability issues, | Day 57. |
| continued attempts to get them to the | If at 24 mg on Day 57, continue that dose |
| 16 mg dose can be made up to Day | for the duration of the treatment period. |
| 78 | If at 12 mg or 16 mg or 20 mg on |
| 24.0 mg at Day 57, Day 64, Day 71, | Day 57, titration to the |
| and Day 78 | corresponding |
| 20.0 mg may be provided on Day 64 | next higher dose level may be |
| and Day 71 if 24 mg is not tolerated | attempted on a weekly basis based on |
| by an individual on Day 57 | the individual's tolerability (e.g., if on |
| Attempts must be made to get to | Day 57, 16 mg dose was administered |
| 24 mg by Day 78 | and tolerated, on Day 64, dose of 20 |
| If tolerability remains a concern for a | mg may be attempted). |
| given individual to reach a dose up to | If not able to tolerate, continue at the |
| 24 mg, they can continue at the | previously tolerated dose level until the |
| 20 mg dose; continued attempts to | following week (e.g., to Day 71), at |
| get them to the 24 mg dose can be | which point another attempt can be |
| made up to Day 78 | made to the next higher dose level. |
| If an individual participant is not able to tolerate | Attempts to the next higher dose level |
| the above titration regimen to 24 mg with the | can continue to be made until Day 78. |
| recommended flexibility and adjustments, a | The dose level achieved at Day 78 |
| minimum dose of 16 mg is required to continue | should be maintained until EOT (Day |
| in this cohort. The dose level achieved at Day 78 | 85). |
| should be maintained until EOT (Day 85). | |

### Example 2: Safety and Efficacy Data of CT-388 Treatment Over Four Weeks

This Example describes data showing that CT-388, a novel once-weekly dual GLP-1 and GIP receptor modulator, was safe, well-tolerated, and produced more than 8% weight loss in 4 weeks in overweight and obese adults in a clinical study performed in accordance with the protocol described in Example 1.

### A. Method

A Phase 1, randomized, placebo-controlled, double-blind study was conducted where single ascending doses (SAD; n=40; 0.5-7.5 mg) and multiple ascending doses (MAD; n=24; 5-12 mg via titration in 3 cohorts) were administered as described in Example 1 to overweight/obese adults.

The primary objective of this study was to investigate the safety and tolerability of CT-388 in overweight or obese, but otherwise healthy adult participants. Secondary objectives of this study were to determine the pharmacokinetic (PK) profile of CT-388 and to investigate the effect of CT-388 on pharmacodynamic (PD) parameters of obesity, e.g., body weight, glycemic control, glucose homeostasis, and insulin sensitivity. A total of 64 participants (men/women: 28/36; median age/BMI: 34 years/33 kg/m²; 40 patients in the SAD arm and 24 patients in the MAD arm) received at least 1 dose of CT-388 or placebo.

The 24 patients in the MAD arm were divided into three cohorts (Cohorts 6, 7, and 8) as shown in Table 5 below (EOT: end of treatment).

TABLE 5
MAD Dosing Regimens

| Population: overweight | | | | | | |
|---|---|---|---|---|---|---|
| or obese otherwise | | | | | | |
| healthy participants | | CT-388 or Placebo SC QW | | | | EOT |
| without T2DM | | Day 1 | Day 8 | Day 15 | Day 22 | Day 29 |
| Overweight | N = 8 (6:2) | Cohort 6 | | | | X |
| (BMI ≥ 25 kg/m²) | | 5 mg | 5 mg | 5 mg | 7.5 mg | |
| Obese | N = 8 (6:2) | Cohort 7 | | | | X |
| (BMI ≥ 30 kg/m²) | | 5 mg | 5 mg | 8 mg | 12 mg | |
| Obese | N = 8 (6:2) | Cohort 8 | | | | X |
| (BMI ≥ 30 kg/m²) | | 5 mg | 8 mg | 12 mg | 12 mg | |

Briefly, participants were randomized 3:1 (6 to CT-388, 2 to placebo, n=8 per cohort). CT-388 was administered once weekly at a dose of 5 mg at baseline in all cohorts and up-titrated up to 12 mg in Cohorts 7 & 8. Oral Glucose Tolerance Test (OGTT) with 75 g glucose solution was performed at baseline (Day −1) and on Day 23. Blood was collected at 15 min prior to OGTT, at OGTT start, and in 30-minute intervals until 120 minutes. Fasting glucose, insulin, C-peptide, and HOMA-IR were measured/calculated at baseline and on Day 23.

Demographics and baseline characteristics of the MAD participants enrolled in this study are shown in Table 6 below.

TABLE 6
MAD Demographics and Baseline Characteristics

| | | | CT-388 | | CT-388 | | CT-388 | |
|---|---|---|---|---|---|---|---|---|
| | | | (5/5/5/7.5 mg) | | (5/5/8/12 mg) | | (5/8/12/12 mg) | |
| | Placebo | | (N = 6) | | (N = 6) | | (N = 6) | |
| Category | (N = 6) | | (Cohort 6) | | (Cohort 7) | | (Cohort 8) | |
| Age, years* | 41.5 | (10.7) | 34.0 | (10.9) | 34.3 | (14.0) | 26.3 | (7.9) |
| Female, n (%) | 4 | (66.7) | 5 | (83.3) | 1 | (16.7) | 2 | (33.3) |
| Race: White, n (%) | 0 | | 0 | | 0 | | 0 | |
| Ethnicity: Hispanic or Latino | 6 | (100) | 6 | (100) | 6 | (100) | 6 | (100) |
| Weight (kg)* | 90.1 | (14.1) | 81.8 | (16.7) | 107.2 | (19.3) | 97.1 | (18.9) |
| BMI (kg/m²)* | 34.0 | (1.1) | 31.3 | (3.8) | 35.6 | (2.8) | 34.8 | (4.0) |

| | | | | |
|---|---|---|---|---|
| HOMA-IR** | 3.8 | 2.2 | 5.0 | 3.9 |
| Fasting Glucose (mg/dL)** | 98.0 | 91.5 | 96.5 | 88.5 |
| HbA1c (%)** | 5.5 | 5.2 | 5.3 | 5.4 |
| Fasting insulin, mIU/L** | 11.7 | 8.9 | 18.9 | 12.7 |
| Fasting C-peptide, ng/mL** | 2.7 | 2.5 | 3.7 | 2.7 |

*Mean (SD) values (within parentheses);
**Median values (within parentheses)

### B. Results

PK profile was investigated over a wide dose range (0.5-12 mg) and supports once-weekly administration. Percent change in body weight from baseline at Day 29 was dose responsive and significantly greater in CT-388 treated participants versus placebo (p<0.0001) across the 3 MAD cohorts with placebo adjusted least square mean difference [95% CI] of −4.8% [−6.3, −3.3], −6.4% [−7.8, −5.0], and −8.5% [−10.4, −6.7]. Mean percent decrease from baseline at Day 23 in fasting glucose (↓ 8-10%), insulin (↓ 20-26%), HOMA-IR (↓ 26-33%), AUC_{0-120 min} glucose (↓ 26-28%) and AUC_{0-120 min} insulin (↓ 33-58%) during oral glucose tolerance tests (OGTTs) were seen in cohorts dosed up to 12 mg of CT-388, suggestive of improved insulin sensitivity. Over the 4-week treatment period, CT-388 was generally well tolerated with no serious adverse events or treatment-related discontinuations in the MAD cohorts. The most frequent side effects reported were gastrointestinal (decreased appetite, nausea, vomiting, diarrhea), which were mostly mild in severity. The data are discussed in detail below.

### B.1. Summary of SAD Cohorts 1-5

### Safety Evaluation

Preliminary and blinded safety and tolerability data indicate that CT-388 was generally well-tolerated with most treatment-emergent adverse events (TEAEs) being GI-related, consistent with the expected AE profile of the incretin-based (GLP-1R agonists and dual GLP-1R/GIPR agonist) drug class, as shown in Table 7A and Table 7B below.

TABLE 7A
Frequency/Type of AEs in SAD Cohorts

| | Cohort 1 | | Cohort 2 | | Cohort 3 | | Cohort 4 | | Cohort 5 | |
|---|---|---|---|---|---|---|---|---|---|---|
| | 0.5 mg/ | | 2 mg/ | | 5 mg/ | | 7.5 mg/ | | 6 mg/ | |
| | Placebo | | Placebo | | Placebo | | Placebo | | Placebo | |
| Categories | (N = 8) | | (N = 8) | | (N = 8) | | (N = 8) | | (N = 8) | |
| TEAEs | 8 | (100.0%) | 8 | (100.0%) | 6 | (75.0%) | 8 | (100.0%) | 6 | (75.0%) |
| Related | 0 | (0.0%) | 6 | (75.0%) | 4 | (50.0%) | 7 | (87.5%) | 4 | (50.0%) |
| TEAEs | | | | | | | | | | |
| Severe | 0 | (0.0%) | 0 | (0.0%) | 0 | (0.0%) | 1 | (12.5%) | 1 | (12.5%) |
| TEAEs | | | | | | | | | | |
| Moderate | 0 | (0.0%) | 1 | (12.5%) | 0 | (0.0%) | 5 | (62.5%) | 2 | (25.0%) |
| TEAEs | | | | | | | | | | |
| Serious | 0 | (0.0%) | 0 | (0.0%) | 0 | (0.0%) | 1 | (12.5%) | 1 | (12.5%) |
| TEAEs | | | | | | | | | | |
| Life- | 0 | (0.0%) | 0 | (0.0%) | 0 | (0.0%) | 0 | (0.0%) | 0 | (0.0%) |
| threatening | | | | | | | | | | |
| TEAE | | | | | | | | | | |
| TEAE | 0 | (0.0%) | 0 | (0.0%) | 0 | (0.0%) | 0 | (0.0%) | 0 | (0.0%) |
| leading to | | | | | | | | | | |
| death | | | | | | | | | | |

TABLE 7B
Specific AEs and Verbatim Terms Reported for SAD Cohorts

| | Cohort 1 | Cohort 2 | Cohort 3 | Cohort 4 | Cohort 5 |
|---|---|---|---|---|---|
| | 0.5 mg/ | 2 mg/ | 5 mg/ | 7.5 mg/ | 6 mg/ |
| | Placebo | Placebo | Placebo | Placebo | Placebo |
| Verbatim Term | (N = 8) | (N = 8) | (N = 8) | (N = 8) | (N = 8) |
| Abdominal | 0 | 0 | 0 | 1 (12.5%) | 0 |
| bloating | | | | | |
| Abdominal cramp | 0 | 2 (25.0%) | 0 | 0 | 0 |
| with loose bowel | | | | | |
| Abdominal cramp | 0 | 0 | 0 | 1 (12.5%) | 0 |
| Abdominal pain | 0 | 0 | 0 | 0 | 1 (12.5%) |
| Abnormal LFTs | 0 | 0 | 0 | 1 (12.5%) | 0 |
| Constipation | 0 | 2 (25.0%) | 0 | 0 | 1 (12.5%) |
| Diarrhea | 0 | 0 | 0 | 2 (25.0%) | 0 |
| Dyspepsia | 0 | 0 | 1 (12.5%) | 0 | 0 |
| Dysphasia | 0 | 0 | 1 (12.5%) | 0 | 0 |
| Early satiety | 0 | 1 (12.5%) | 0 | 0 | 0 |
| Emesis | 0 | 0 | 2 (25.0%) | 0 | 0 |
| Exacerbated | 0 | 0 | 0 | 1 (12.5%) | 0 |
| GERD | | | | | |
| Fatigue | 0 | 2 (25.0%) | 0 | 0 | 0 |
| Headache | 0 | 1 (12.5%) | 0 | 2 (25.0%) | 1 (12.5%) |
| Indigestion | 0 | 0 | 1 (12.5%) | 0 | 0 |
| Lethargy | 0 | 1 (12.5%) | 0 | 0 | 0 |
| Loose bowel | 0 | 0 | 0 | 0 | 1 (12.5%) |
| motion | | | | | |
| Loose stools | 0 | 0 | 0 | 1 (12.5%) | 0 |
| Loss of Appetite | 0 | 2 (25.0%) | 1 (12.5%) | 1 (12.5%) | 1 (12.5%) |
| Microscopic | 0 | 0 | 1 (12.5%) | 0 | 0 |
| hematuria | | | | | |
| Nausea | 0 | 0 | 0 | 6 (75.0%) | 2 (25.0%) |
| Reduced appetite | 0 | 0 | 0 | 2 (25.0%) | 1 (12.5%) |
| Reduced bowel | 0 | 0 | 0 | 1 (12.5%) | 0 |
| motion | | | | | |
| Reflux | 0 | 0 | 0 | 2 (25.0%) | 0 |
| Throat discomfort | 0 | 0 | 0 | 1 (12.5%) | 0 |
| Vomiting | 0 | 0 | 0 | 3 (37.5%) | 2 (25.0%) |

Most of these AEs were considered mild in nature. Across Cohorts 1-3, generally no clinically significant AEs nor changes in vital signs, ECG, physical exam, or laboratory findings were reported.

### Pharmacokinetics

Time-concentration profiles following a single dose SC administration of CT-388 to participants in Cohort 1 (0.5 mg), Cohort 2 (2 mg), Cohort 3 (5 mg), Cohort 4 (7.5 mg) and Cohort 5 (6 mg) are shown in Table 8 below.

TABLE 8
PK Parameters from SAD Cohorts

| | | Cₘₐₓ | AUC_{0-∞} | t_{1/2} range | CL/F | Vz/F | MRTₗₐₛₜ |
|---|---|---|---|---|---|---|---|
| Cohort | Dose (mg) | (ng/ml) | (h · ng/mL) | (hour) | (mL/h) | (L) | (hour) |
| 1 | 0.5 | 52.4 ± 10.0 | 12890 ± 1093 | 91.9-170.7 | 39 ± 3.2 | 5.8 ± 0.7 | 128 ± 8.2 |
| 2 | 2 | 216.0 ± 51.4 | 47350 ± 10590 | 108.5-142.1 | 44 ± 12.9 | 7.7 ± 1.8 | 122 ± 5.6 |
| 3 | 5 | 831.0 ± 262.6 | 140400 ± 41660 | 107.7-146.2 | 38.4 | 6.8 ± 2.1 | 114 ± 2.3 |
| 5 | 6 | 551.6 ± 80.1 | 99970 ± 15000 | 101.4-141 | 59.4 ± 6.3 | 11 ± 1.2 | 121.9 ± 9.6 |
| 4 | 7.5 | 1165.0 ± 109.7 | 235000 ± 39000 | 117.1-151.1 | 32.8 | 6.6 ± 1.3 | 119 ± 5.3 |

* CL/F = oral clearance;
MRTₗₐₛₜ = mean residence time from the time of dosing to the time of the last measurable concentration;
t_{1/2} = half-life (shown as range of minimum to maximum);
Vz/F = volume of distribution.
All values are mean ± standard deviation.

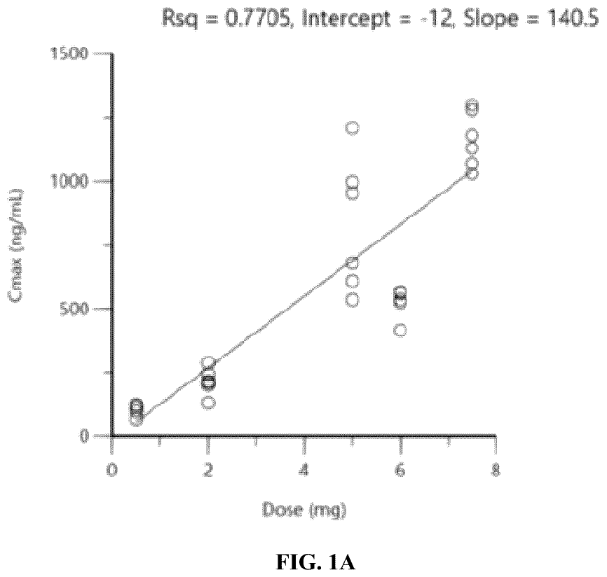

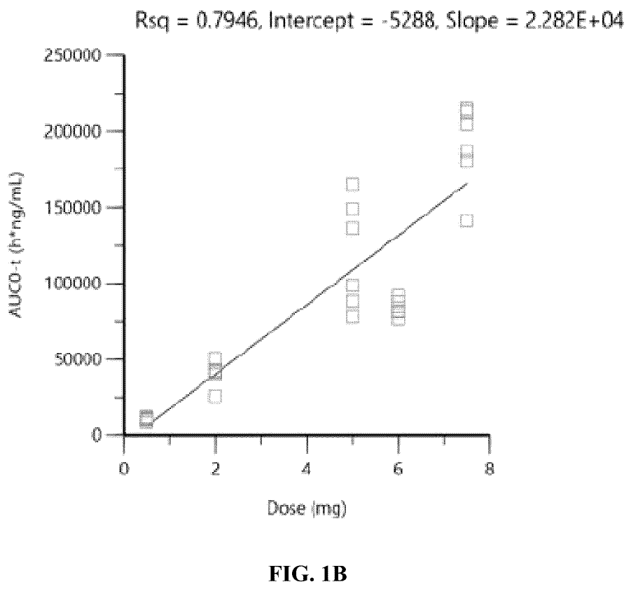

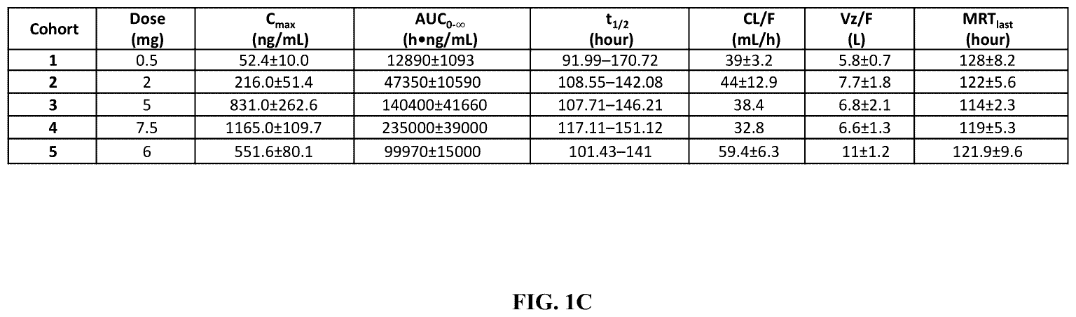

A generally proportional increase in Cₘₐₓ and AUC₀₋ₜ was observed with increasing dose, except for Cohort 5 which appeared to be less than dose-proportional (FIGS. 1A and 1B). Samples from Cohort 5 were re-analyzed to verify the initial results which yielded similar findings to that originally observed. Tₘₐₓ occurred after 24 hours in all except two individual cases. The t_{1/2} (median about 120 hours) is consistent with a once-weekly dosing regimen for CT-388. Preliminary PK parameters from each of Cohorts 1-5 is summarized in FIG. 1C.

### B.2. Summary of MAD Cohorts 6-8

### Safety Evaluation

A summary of TEAEs associated with CT-388 administration in the MAD study is shown in Table 9 below.

TABLE 9
TEAE in MAD Cohorts

| | | | CT-388 | CT-388 | | CT-388 | |
|---|---|---|---|---|---|---|---|
| | | | (5/5/5/7.5 mg) | (5/5/8/12 mg) | | (5/8/12/12 mg) | |
| | | Placebo | (N = 6) | (N = 6) | | (N = 6) | |
| Category | Statistic | (N = 6) | (cohort 6) | (cohort 7) | | (cohort 8) | |
| Subjects with at least 1 | n (%) | 5 (83.3) | 6 (100) | 6 | (100) | 6 | (100) |
| TEAE | | | | | | | |
| Serious TEAEs | # Events | 0 | 0 | 0 | | 0 | |
| Subjects discontinued | n (%) | 0 | 0 | 0 | | 0 | |
| due to TEAE | | | | | | | |
| Fatal TEAEs | # Events | 0 | 0 | 0 | | 0 | |
| Drug-related^{[a]} TEAEs | # Events | 10 | 33 | 18 | | 25 | |
| Severity: | | | | | | | |
| Grade 1 | n (%) | 4 (66.7) | 6 (100) | 6 | (100) | 6 | (100) |
| Grade 2 | n (%) | 4 (66.7) | 6 (100) | 1 | (16.7) | 3 | (50.0) |

| | | | | | |
|---|---|---|---|---|---|
| Grade 3 | n (%) | 0 | 0 | 0 | 0 |

Gastrointestinal (GI) TEAEs are shown in Table 10 below.

TABLE 10
GI TEAE in MAD Cohorts

| | | | CT-388 | | CT-388 | CT-388 |
|---|---|---|---|---|---|---|
| | | | (5/5/5/7.5 mg) | | (5/5/8/12 mg) | (5/8/12/12 mg) |
| | Placebo | | (N = 6) | | (N = 6) | (N = 6) |
| Preferred Term, n (%) | (N = 6) | | (Cohort 6) | | (Cohort 7) | (Cohort 8) |
| GI TEAEs (Total) | 3 | (50) | 6 | (100) | 4 (66.7) | 5 (83.3) |
| Abdominal Distention | 1 | (16.7) | 3 | (50.0) | 0 | 0 |
| Constipation | 0 | | 3 | (50.0) | 0 | 1 (16.7) |
| Diarrhea | 3 | (50.0) | 2 | (33.3) | 2 (33.3) | 3 (50.0) |
| Dyspepsia | 1 | (16.7) | 1 | (16.7) | 0 | 0 |
| Eructation | 0 | | 0 | | 2 (33.3) | 1 (16.7) |
| Gastroesophageal Reflux Disease | 0 | | 0 | | 0 | 2 (33.3) |
| Nausea | 1 | (16.7) | 6 | (100) | 1 (16.7) | 3 (50.0) |

| | | | | | |
|---|---|---|---|---|---|
| Vomiting | 0 | 5 | (83.3) | 2 (33.3) | 2 (33.3) |

Severity of gastrointestinal TEAEs is shown in Table 11 below.

TABLE 11
Severity of GI TEAEs in MAD Cohorts

| | | CT-388 | | CT-388 | CT-388 |
|---|---|---|---|---|---|
| | | 5/5/5/7.5 mg | | 5/5/8/12 mg | 5/8/12/12 mg |
| | Placebo | (N = 6) | | (N = 6) | (N = 6) |
| TEAE, n (%) | (N = 6) | (Cohort 6) | | (Cohort 7) | (Cohort 8) |
| Vomiting* | 0 | 5 | (83.3) | 2 (33.3) | 2 (33.3) |
| Grade 1 | 0 | 2 | (33.3) | 2 (33.3) | 2 (33.3) |
| Grade 2 | 0 | 3 | (50.0) | 0 | 1 (16.7) |
| Grade 3 | 0 | 0 | | 0 | 0 |
| Nausea* | 1 (16.7) | 6 | (100) | 1 (16.7) | 3 (50.0) |
| Grade 1 | 1 (16.7) | 4 | (66.7) | 1 (16.7) | 2 (33.3) |
| Grade 2 | 0 | 4 | (66.7)** | 0 | 1 (16.7) |
| Grade 3 | 0 | 0 | | 0 | 0 |
| Diarrhea* | 3 (50.0) | 2 | (33.3) | 2 (33.3) | 3 (50.0) |
| Grade 1 | 3 (50.0) | 2 | (33.3) | 2 (33.3) | 3 (50.0) |
| Grade 2 | 0 | 0 | | 0 | 0 |
| Grade 3 | 0 | 0 | | 0 | 0 |
| Constipation* | 0 | 3 | (50.0) | 0 | 1 (16.7) |
| Grade 1 | 0 | 3 | (50.0) | 0 | 1 (16.7) |

| | | | | |
|---|---|---|---|---|
| Grade 2 | 0 | 0 | 0 | 0 |
| Grade 3 | 0 | 0 | 0 | 0 |

*Each subject contributes once to each of the incidence rates.
**Same subject experienced both mild and moderate events.

In summary, CT-388 12 mg dosed with a weekly up-titration regimen over 4 weeks was generally well-tolerated.

### Efficacy

### Weight Loss

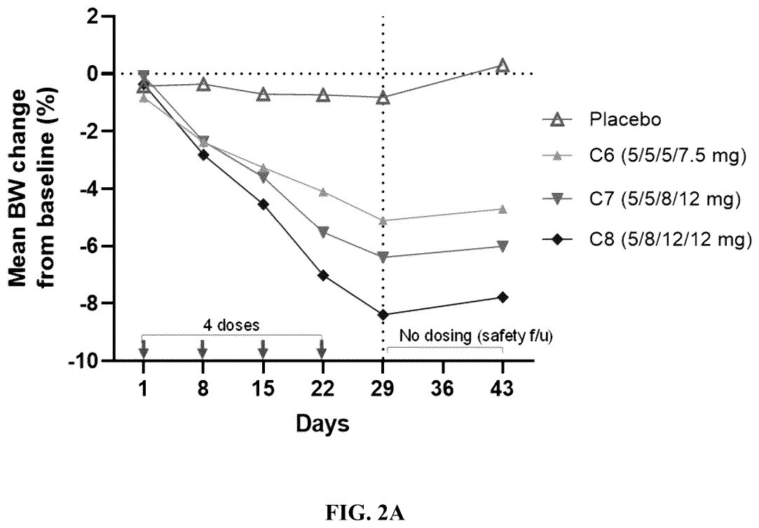

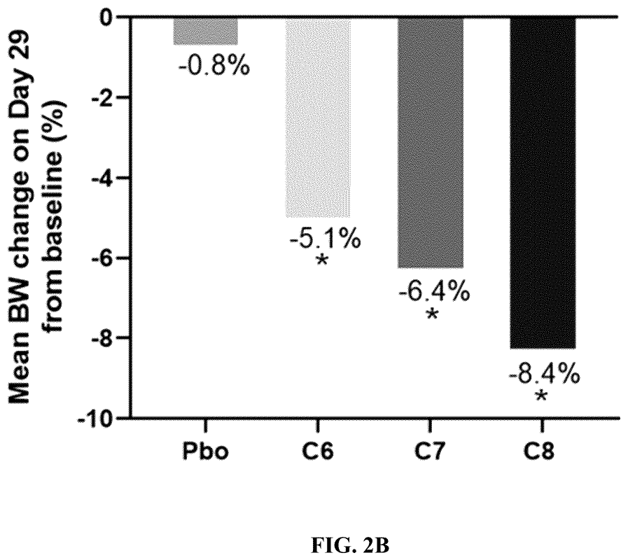

Enrolled patients had a mean body weight (BW) of 94 kg. A robust dose response in BW was observed following 4 weeks of CT-388 dosing. All dose arms yielded clinically and statistically significant weight loss (WL) compared to placebo. Specifically, after 4 weeks of CT-388 administration, placebo-adjusted (adj) WL (LS Means) were 3.7 kg, 6.0 kg, and 7.4 kg for Cohorts 6, 7, and 8, respectively (p<0.001 for all). WL effects persisted for up to 2 weeks following CT-388 discontinuation (FIGS. 2A and 2B). CT-388 effect on BW was noted after the first dose and improved over time. Maximum beneficial effect was seen in obese participants, with WL up to 7.7 kg (about 17 lbs), 8.4%.

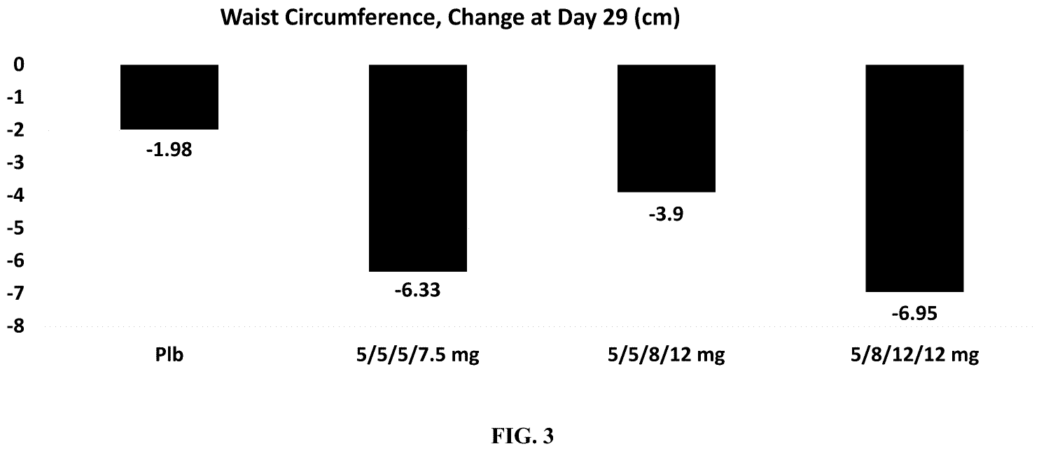

A decrease in waist circumference was observed following CT-388 therapy, which correlates with decrease in visceral fat (FIG. 3).

### Glucose and Insulin Response

On Day −1, OGTT fasting glucose was 94 mg/dL, 99 mg/dL, and 98 mg/dL for Cohorts 6, 7, and 8, respectively; it was 95 mg/dL for patients in the placebo arm. OGTT fasting insulin on Day −1 was 10 mIU/L, 19 mIU/L, and 41 mIU/L for Cohorts 6, 7, and 8, respectively; it was 12 mIU/L for patients in the placebo arm. OGTT fasting C-peptide on Day −1 was 2.73 ng/mL for Cohort 6, 3.55 ng/mL for Cohort 7, 4.18 ng/mL for Cohort 8, and 12.22 ng/mL for placebo. HOMA-IR on Day −1 was 2.2, 4.7, and 13.2 for Cohorts 6, 7, and 8, respectively; it was 2.8 for patients in the placebo arm.

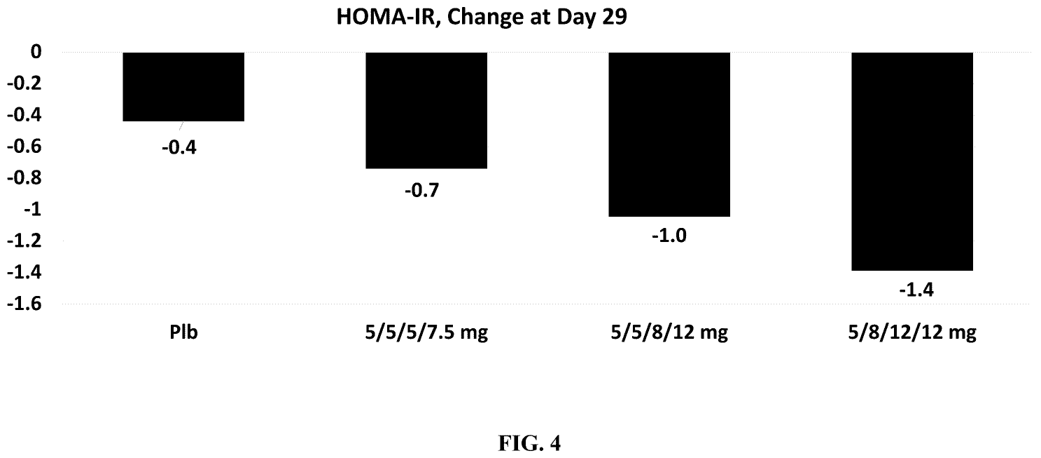

After four CT-388 doses, placebo-adjusted differences in Cohorts 6, 7, and 8 vs. the placebo arm were: fasting glucose −13/−5/−15 mg/dL; fasting insulin: −1/2/−4 mIU/L; fasting C-Peptide −0.2/0.5/−0.9 ng/mL; and HOMA-IR: −0.46/0.58/−1. AUC_{0-120 min} placebo-adjusted decreases in Cohorts 6, 7, and 8 were 66 mg*h/dL, 97 mg*h/dL, and 88 mg*h/dL, respectively, for glucose (p<0.001 for all); 5 mIU*h/L, 127 mIU*h/L, and 71 mIU*h/L, respectively, for insulin (p<0.5 for Cohort 6); and 0.6 ng*h/mL, 0.7 ng*h/mL, and 5.1 ng*h/mL, respectively, for C-peptide (p<0.05 for Cohorts 7/8). Decreases in HOMA-IR (markers of insulin sensitivity) were observed following CT-388 therapy, suggesting improved insulin sensitivity (FIG. 4).

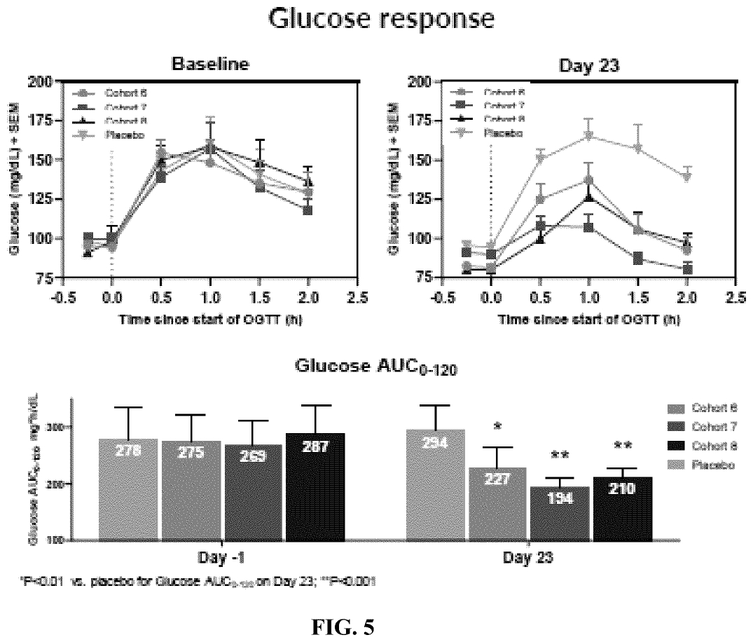

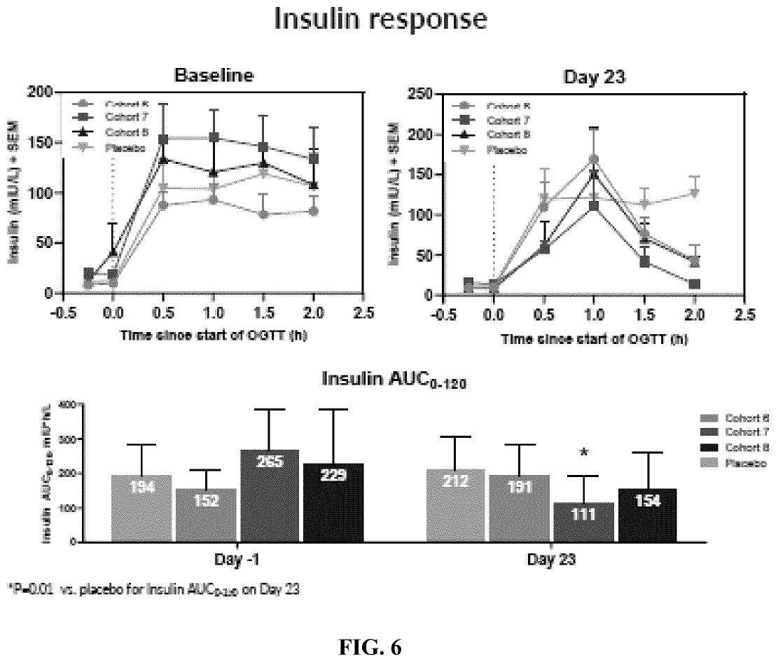

In summary, on Day 23, glucose AUC_{0-120 min} was significantly reduced in all CT-388 cohorts compared to placebo (P<0.001 for all); and insulin AUC_{0-120 min} and C-peptide AUC_{0-120 min} were lower in participants with baseline BMI≥30 kg/m² (Cohorts 7 & 8). See FIGS. 5 and 6. Peak glucose excursion (Cₘₐₓ) was reduced by 16-31% across all CT-388 cohorts vs. placebo (P<0.05) accompanied by decreases in Cm. of insulin (20-55% in Cohorts 7 & 8) and C-peptide (17-39% in Cohorts 7 & 8). Further, on Day 23, improvements were observed in fasting glucose in all CT-388 cohorts compared to placebo (P=0.001 for Cohorts 6 & 8). Fasting insulin, C-peptide, and HOMA-IR were also significantly lower for Cohort 8 compared to placebo on Day 23 (P<0.05).

TABLE 12
HOMA-IR

| | HOMA-IR* | Ratio | |
|---|---|---|---|
| | (Day 23) | (vs. placebo) | 95% CI |
| Cohort 6 | 1.75 | 0.65 | 0.33, 1.31 |
| Cohort 7 | 2.37 | 0.89 | 0.44, 1.80 |
| Cohort 8 | 1.15 | 0.43** | 0.21, 0.87 |
| Placebo | 2.67 | | |

*Values represent geometric mean;
**P = 0.02 vs. placebo for HOMA-IR on Day 23

### Pharmacokinetic (PK)

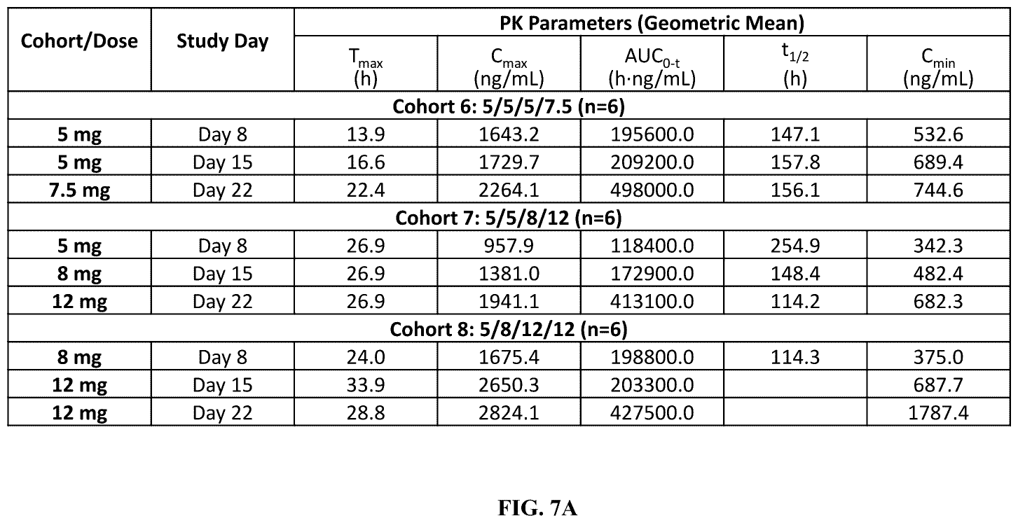

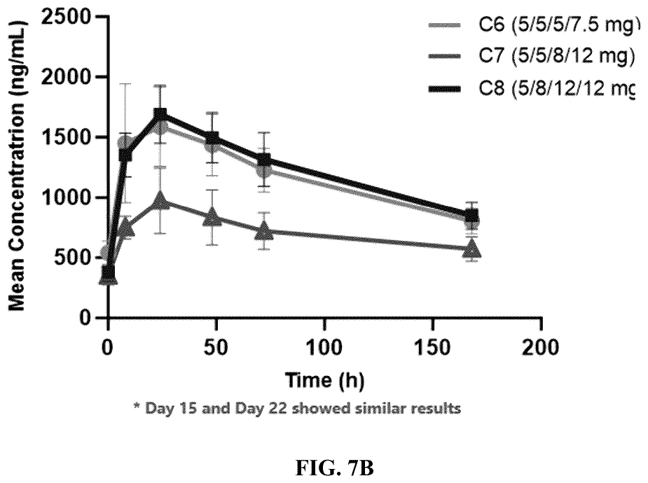

Profiles of mean (±SD) plasma concentrations (ng/ml) vs. time (hours) for Cohort 6 (5/5/5/7.5 mg), Cohort 7 (5/5/8/12 mg), and Cohort 8 (5/8/12/12 mg) upon dosing with CT-388 or placebo on Day 8 are shown in FIGS. 7A and 7B. Generally proportional increase in Cₘₐₓ and AUC₀₋ₜ within each cohort was observed. PK data are consistent with once-weekly (QW) dosing. CT-388 plasma exposure appeared lower in participants with a higher baseline body weight (BW).

### Conclusion

CT-388 at a starting dose of 5 mg and up-titrated to 12 mg within 4 weeks showed a favorable tolerability profile in both overweight and obese participants. Tolerability appeared more favorable in obese participants compared to overweight participants. Most common TEAEs were GI-related, consistent with the incretin class, with no temporal patterns for AEs around dose up-titration periods. PK profile supports once weekly (QW) dose administration. CT-388 dosed at 5/8/12/12 mg produced 8.4% weight loss (7.7 kg, about 17 lbs) accompanied by decrease in waist and hip circumference and improvement in markers of insulin sensitivity (HOMA-IR). Given the highest weight loss and favorable tolerability, obese patients were shown to benefit to a marked degree from treatment with CT-388.

In summary, CT-388 delivers clinically meaningful weight loss and metabolic control with a favorable tolerability profile. Administration of CT-388 to insulin-resistant overweight or obese patients induced clinically meaningful WL accompanied by improved insulin sensitivity (HOMA-IR) and glucose homeostasis. These findings suggest that CT-388 may reduce the risk of T2D in obese individuals, and have a beneficial effect on glucose metabolism and induce disease remission in patients with T2D. These data warrant further clinical evaluation of CT-388, possibly with minimal to no titration, for the treatment of obesity, T2DM, and other weight-related comorbidities.

### Example 3: Safety and Efficacy Data of CT-388 Treatment In Cohorts 11 and 12

### Trial Design

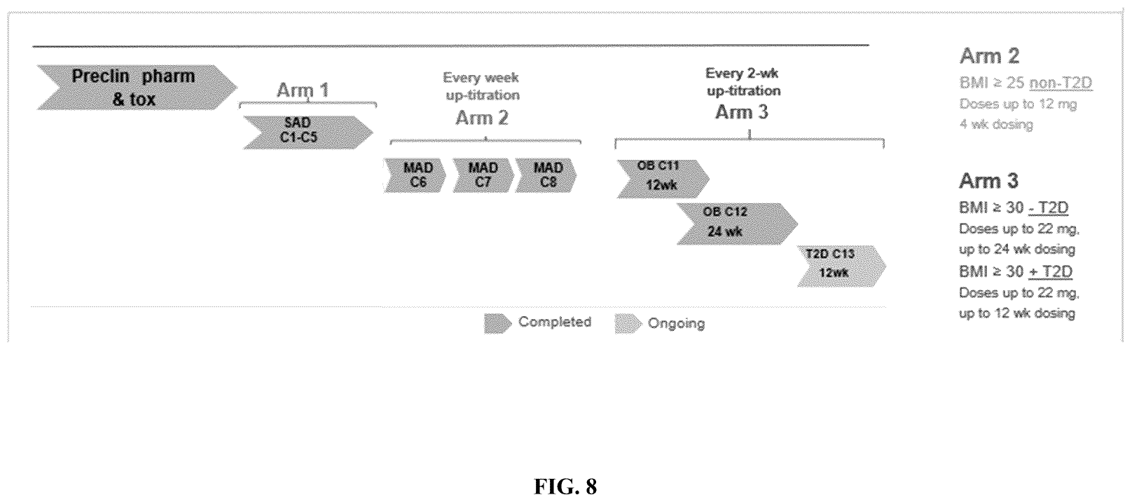

CT-388-101 is a first in human Phase 1, double-Blinded, placebo controlled, single center study. FIG. 8 shows the seamless phase 1/2 design of the trial. In arm 3, patients having a BMI≥30 without T2D were treated with doses up to 22 mg for up to 24 weeks, while patients having a BMI≥30 with T2D were treated with doses up to 22 mg for up to 12 weeks.

In all, a total of 129 participants were enrolled in CT-388-101 and received at least one dose of CT-388 or placebo. In the SAD phase (n=40:3:1 ratio; overweight or obesity without T2D), 0.5 mg to 7.5 mg CT-388 were dosed. In the MAD QW phase (n=24:3:1 ratio; overweight or obesity without T2D), up to 12 mg CT-388 was dosed over four weeks. In the MD QW phase (n=60; 4:1 ratio; obesity with or without T2D), up to 22 mg was dosed over 12-24 weeks, and 45 non-T2D patients were enrolled.

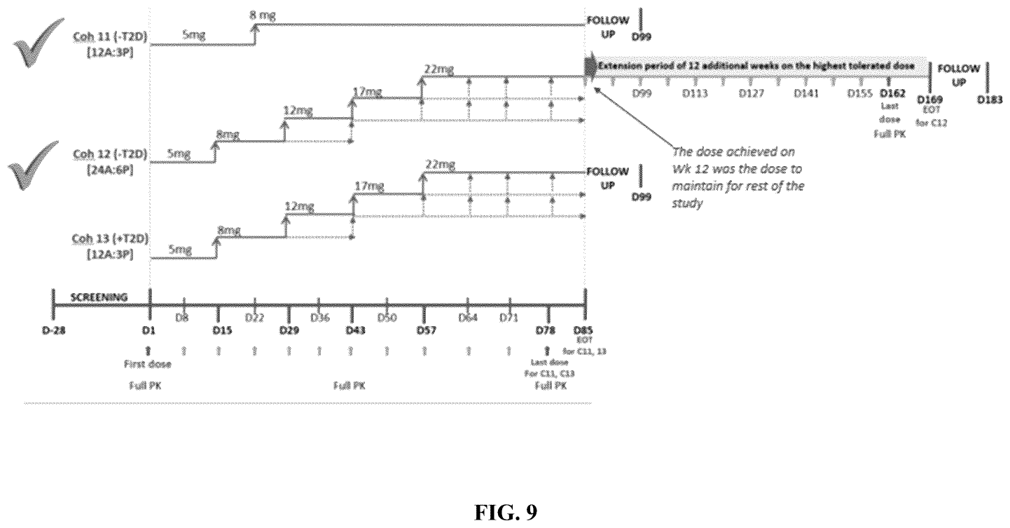

FIG. 9 shows the CT-388-101 MD phase Design. Cohorts 11 and 12 included participants who were obese without T2DM and cohort 13 included patients who were obese with T2DM. Endpoints of this study were safety, including TEAEs, labs, vitals and ECGs; PK, including CT-388 PK concentrations and parameters; and key PD endpoints, including week 12 (D85) and week 24 (D169):
a. body weight: % change; absolute change;
b. change in HbA1c, fasting glucose, fasting insulin, HOMA-IR; and
c. waist and hip circumference.

### Demographic and Baseline Characteristics

The majority of CT-388 patients were females and were in obesity class 2-3 (BMI≥35 kg/m²). Other baseline characteristics between placebo and CT-388 patients were similar (Table 13). Although baseline mean HbA1c and fasting glucose were within normal limits, ˜50% of patients met pre-diabetic/diabetic status per ADA definition (Post hoc derivation based on the ADA criteria, with only glucose at 120 min post OGTT (Day −1) meeting the diagnosis threshold (>200 mg/dL)). Baseline is defined as the last observation prior to the first dose of the study drug.

TABLE 13
CT-388-101 MD Demographic and Baseline Characteristics

| | | Planned Target | Planned Target | | |
|---|---|---|---|---|---|
| | Pooled | CT-388 Dose | CT-388 Dose | CT-388 | |
| | Placebo | (8 mg)^{a} | (22 mg)^{b} | Total | Total |
| | (N = 10) | (N = 12) | (N = 24) | (N = 36) | (N = 46) |
| Age (years), Mean | 37.3 | 37.4 | 32.3 | 34.0 | 34.7 |
| (SD) | (12.66) | (9.80) | (9.07) | (9.50) | (10.20) |
| Sex, females n (%) | 3 | 10 | 15 | 25 | 28 |
| | (30.0) | (83.3) | (62.5) | (69.4) | (60.9) |

Ethnicity, n (%)

| | | | | | |
|---|---|---|---|---|---|
| Hispanic or Latino | 10 | 12 | 24 | 36 | 46 |
| | (100) | (100) | (100) | (100) | (100) |
| Body Weight (kg), | 98.2 | 96.7 | 107.9 | 104.2 | 102.9 |
| Mean (SD) | (13.26) | (18.16) | (19.04) | (19.24) | (18.15) |
| BMI (kg/m²), Mean | 34.6 | 37.5 | 38.6 | 38.2 | 37.4 |
| (SD) | (3.03) | (5.86) | (5.59) | (5.62) | (5.36) |
| 30 to 35 | 4 | 4 | 7 | 11 | 15 |
| | (40.0) | (33.3) | (29.2) | (30.6) | (32.6) |
| 35 to 40 | 6 | 6 | 8 | 14 | 20 |
| | (60.0) | (50.0) | (33.3) | (38.9) | (43.5) |
| >40 | 0 | 2 | 9 | 11 | 11 |
| | | (16.7) | (37.5) | (30.6) | (23.9) |
| Fasting Glucose | 89.6 | 89.3 | 91.1 | 90.5 | 90.3 |
| (mg/dL), Mean (SD) | (7.56) | (8.06) | (7.19) | (7.42) | (7.38) |
| HbA1c, Mean (SD) | 5.5 | 5.3 | 5.4 | 5.4 | 5.4 |
| | (0.32) | (0.26) | (0.33) | (0.31) | (0.31) |

Glycemic Status

| | | | | | |
|---|---|---|---|---|---|
| Normoglycemic | 5 | 4 | 15 | 20 | 25 |
| | (50.0) | (33.3) | (62.5) | (55.6) | (54.3) |
| Prediabetes | 5 | 7 | 7 | 13 | 18 |
| | (50.0) | (58.3) | (29.2) | (36.1) | (39.1) |
| Diabetes | 0 | 1 | 2 | 3 | 3 |
| | | (8.3)* | (8.3)* | (8.3) | (6.5) |

^{a}Cohort 11 titration path: 5 mg for 3 weeks then 8 mg for 9 weeks.
^{b}Cohort 12 titration path: 5 mg, 8 mg, 12 mg and 17 mg, 2 weeks each in this order, then 22 mg from Week 8 to Week 24.

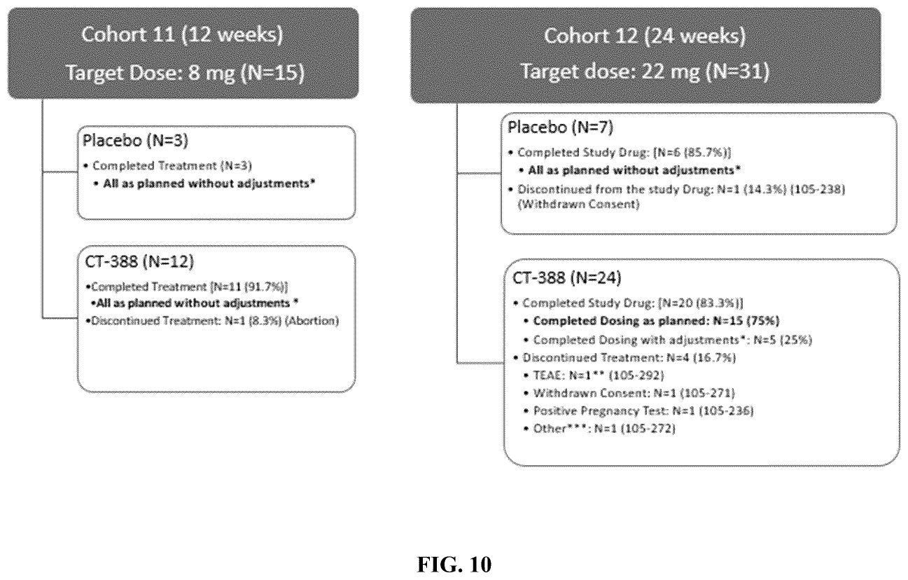

FIG. 10 shows the subject dispositions in the CT-388-101 MD trial. In patients treated with 8 mg, all patients were dosed per plan. In patients treated with 22 mg, 75% of patients were dosed per planned path and completed treatment on 22 mg, while 25% had dose down-titration and completed treatment on 12 mg. There were no patients who completed treatment on 17 mg.

### Efficacy

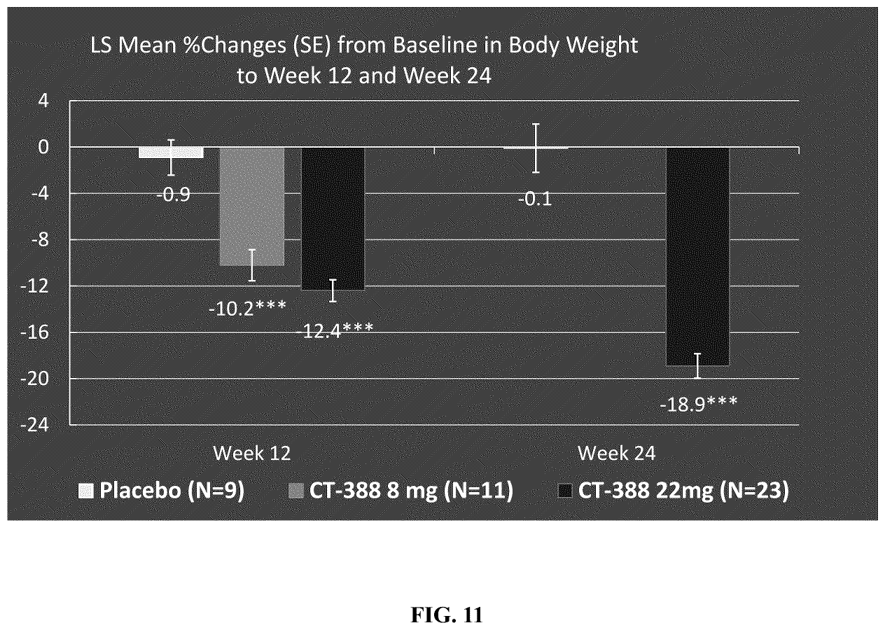

At week 12, Both CT-388 8 mg and 22 mg led to robust, statistically significant, dose dependent, and clinically meaningful weight loss. At week 24, CT-388 resulted in 18.8% placebo-adjusted weight loss, confirming additional decrease in body weight with prolonged treatment (FIG. 11, Table 14).

TABLE 14
Placebo-Adjusted LSMs of % Change in Weight Loss and 95% CI

| | CT-388 8 mg vs | CT-388 22 mg vs. |
|---|---|---|
| | Placebo | Placebo |
| Week 12 | −9.3 | −11.5 |
| | (−13.40, −5.22) | (−15.11, −7.88) |
| Week 12 (w.o. | −10.4 | −12.6 |
| Subject 105-136)$ | (−14.44, −6.29) | (−16.20, −8.93) |
| Week 24 | N/A | −18.8 |
| | | (−23.60, −14.00) |

$Subject 105-136 (Cohort 11, PBO) have PK concentrations at 605 ng/mL starting at Week 11 and going down to 33.8 ng/mL at Week 13, it is suspected the subject took an active drug at Week 10. This subject had BLQ values at trough starting from Day 1 to Week 8 predose. A sensitivity analysis by excluding this subject was performed.

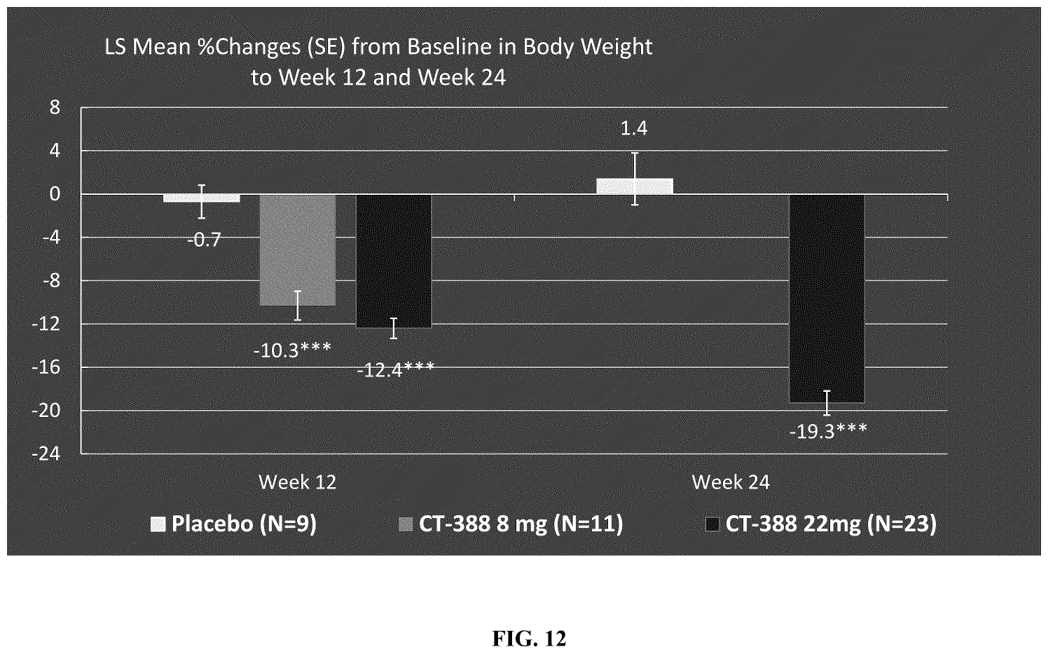

When adjusted to sex and weight at BSL, CT-388 22 mg resulted in −20.7% placebo-adjusted weight loss at Week 24 (FIG. 12, Table 15).

TABLE 15
Placebo-Adjusted LSMs of % Change in Weight Loss and 95% CI

| | CT-388 8 mg vs | CT-388 22 mg vs. |
|---|---|---|
| | Placebo | Placebo |
| Week 12 | −9.6 | −11.8 |
| | (−13.73, −5.56) | (−15.36, −8.15) |
| Week 12 (w.o. | −10.9 | −13 |
| Subject 105-136)$ | (−14.91, −6.81) | (−16.57, −9.37) |
| Week 24 | N/A | −20.7 |
| | | (−26.43, −14.90) |

$Subject 105-136 (Cohort 11, PBO) have PK concentrations at 605 ng/mL starting at Week 11 and going down to 33.8 ng/mL at Week 13, it is suspected the subject took an active drug at Week 10. This subject had BLQ values at trough starting from Day 1 to Week 8 predose. A sensitivity analysis by excluding this subject was performed.

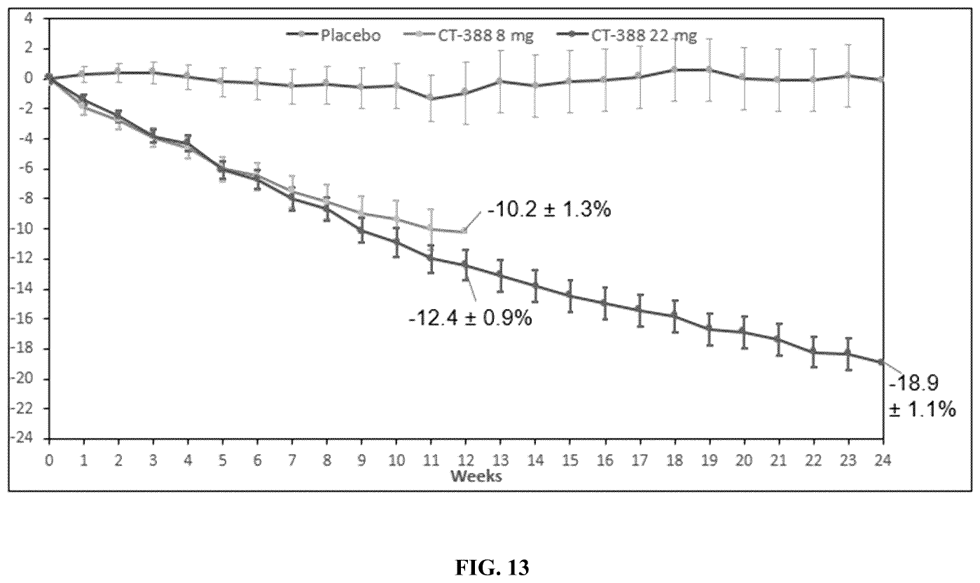

CT-388 led to robust and clinically meaningful weight loss within 12 and 24 weeks of treatment (FIG. 13, Table 16). Weight loss started after the first dose of CT-388 and continue to decrease over the treatment period. No plateau was observed at 8 mg at week 12 or 22 mg at week 24.

TABLE 16
Final Unblinded Evaluation of Weight Loss by Treatment
Groups by Using MMRM Model to Account for the
Effect of Baseline Body Weight Effect

| | At week 12 | At week 24 |
|---|---|---|
| | LSM ± SE | LSM ± SE |
| CT-388 8 mg | −10.2 ± 1.3% | N/A |
| | (n = 11) | |
| CT-388 22 mg | −12.4% ± 0.9% | −18.9 ± 1.1%% |
| | (n = 23) | (n = 20) |
| Placebo | −0.9 ± 1.5% | −0.1 ± 2.1% |
| | (n = 9) | (n = 5) |

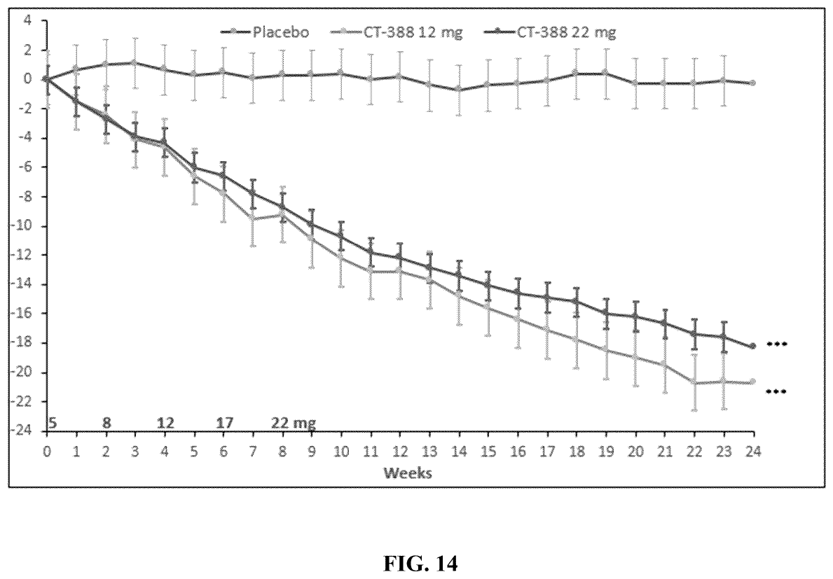

Weight loss was evaluated according to unblinded treatment groups by using MMRM model to account for the effect of baseline weight (FIG. 14, Table 17). Observed weight values were measured within 8 days of the last dose of the study drug. Regardless of the final dose, participants who deviated from planned up-titration path had similar weight loss compared to participants who followed the path. The main reason for dose down-titration were tolerability symptoms, which were managed with dose modification that allowed patients to complete the study and still lose a significant amount of weight.

TABLE 17
Final Unblinded Evaluation of Weight Loss in Cohort
12 By Actual Final Dose Using MMRM Model to Account
for the Effect of Baseline Weight

| | At week 12 | At week 24 |
|---|---|---|
| CT-388 22 mg | −12.20% | −18.30% |
| | (CI 95% −14.22; −10.24) | (CI 95% −20.35, −16.29) |
| CT-388 12 mg* | −13.10% | −20.70% |
| | (CI 95% −16.95; −9.19) | (CI 95% −24.54, −16.79) |
| Placebo | −0.90% | −0.10% |

For 22 mg: n = 16 at W 12, n = 20 at W 24
For 12 mg: n = 7 (n = 3 @17 mg & n = 4 @12 mg) at W 12 and n = 5 at W 24
For Pbo, n = 9 at W 12, n = 5 at W 24
*Participants who deviated from planned titration path had baseline weight ranging from 77 kg to 120 kg

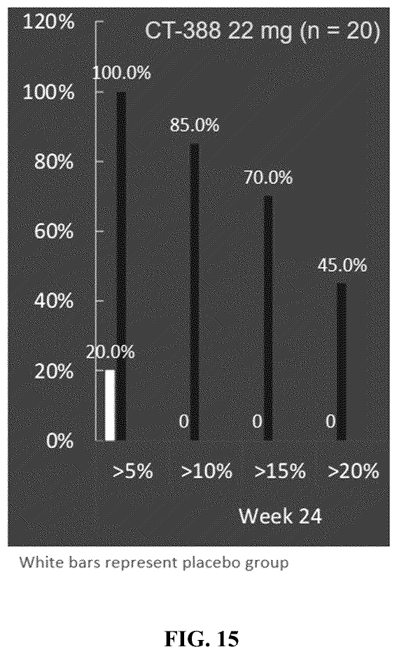

By Week 24, 85% of pts treated with CT-388 22 mg achieved >10% WL, and 45% pts had WL above 20% (FIG. 15).

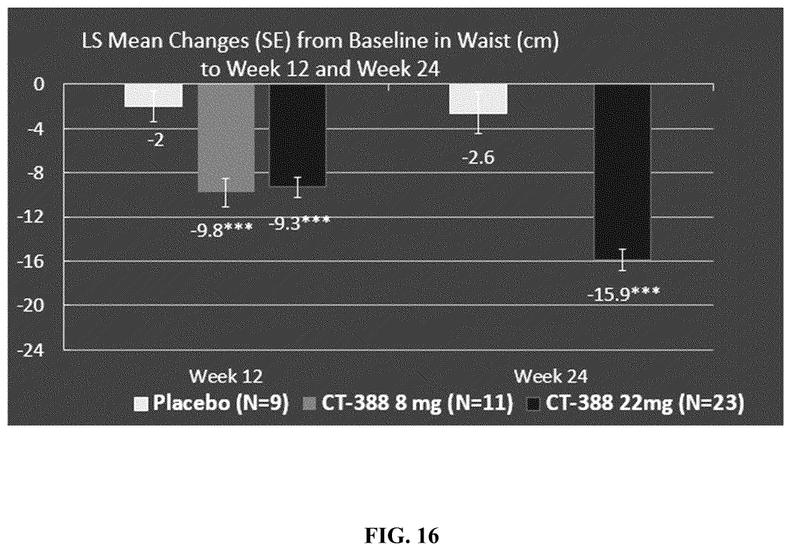

By Week 12, CT-388 8 mg and 22 mg led to significant and clinically meaningful waist circumference reduction of about 10 cm (FIG. 16, Table 18). With prolonged treatment, CT-388 22 mg led to additional decrease in waist circumference of −13.7 cm at week 24. Decreases in waist circumferences suggests a decrease in visceral fat and translates into decrease by about 2-3 clothing sizes.

TABLE 18
Placebo-adjusted LSMs of Change in
Waist Circumference (cm) and 95% CI

| | CT-388 8 mg vs | CT-388 22 mg vs. |
|---|---|---|
| | Placebo | Placebo |
| Week 12 | −7.8 | −7.3 |
| | (−11.70, −3.89) | (−10.69, −3.83) |
| Week 12 (w.o. | −8.6 | −8 |
| Subject 105-136)$ | (−12.53, −4.60) | (−11.57, −4.53) |
| Week 24 | N/A | −13.4 |
| | | (−17.82, −8.95) |

$Subject 105-136 (Cohort 11, PBO) have PK concentrations at 605 ng/mL starting at Week 11 and going down to 33.8 ng/mL at Week 13, it is suspected the subject took an active drug at Week 10. This subject had BLQ values at trough starting from Day 1 to Week 8 predose. A sensitivity analysis by excluding this subject was performed.

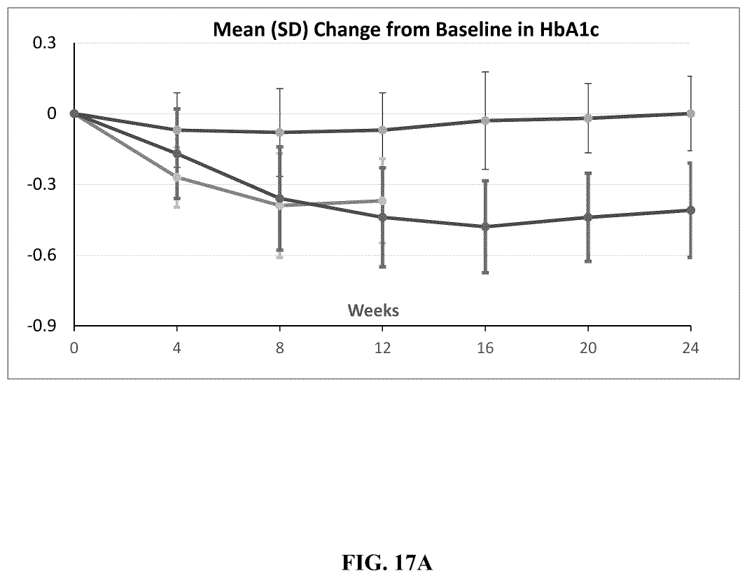

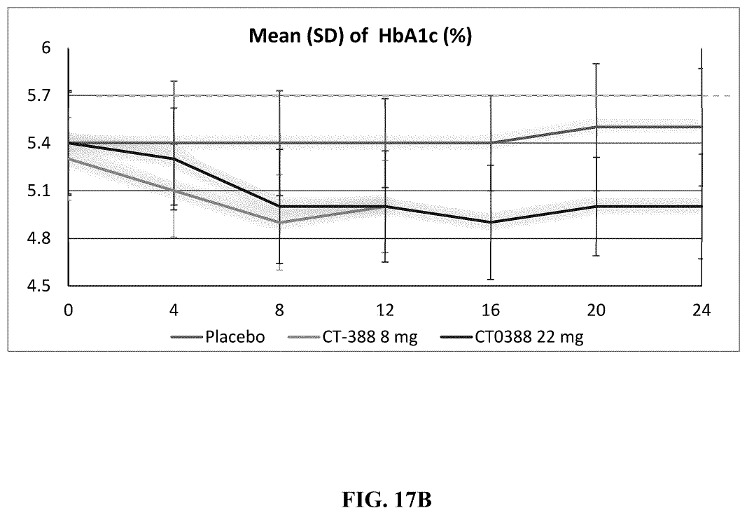

CT-388 8 mg and 22 mg showed potential beneficial effect on HbA1c even in participants with normal HbA1c (<5.7%) at baseline (FIGS. 17A-B, Table 19).

TABLE 19
PBO-adjusted LSMs of Change in HbA1C (%) and 95% CI

| | CT-388 8 mg vs Placebo | CT-388 22 mg vs. Placebo |
|---|---|---|
| Week 12 | −0.3 | −0.4 |
| | (−0.50, −0.16) | (−0.53, −0.24) |
| Week 24 | N/A | −0.4 |
| | | (−0.63, −0.26) |

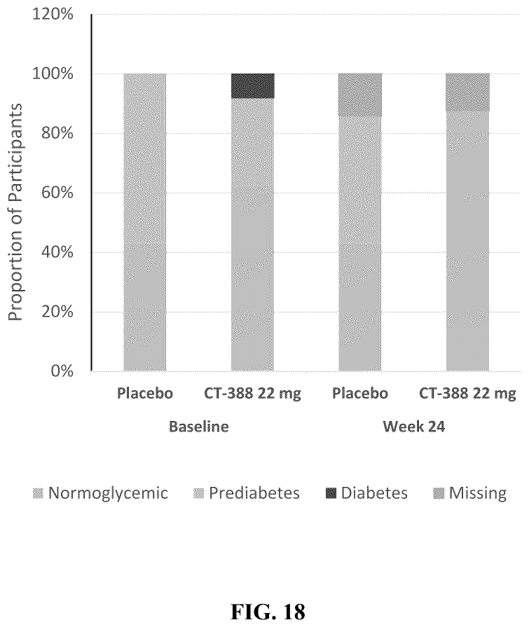

Based on the three ADA criteria, all CT-388 participants were able to normalize their glucose metabolism (FIG. 18, Table 20, Table 21). At baseline, 37.5% patients assigned to CT-388 22 mg were pre-diabetic/diabetic. At week 24, zero participants who completed CT-388 treatment were pre-diabetic/diabetic. As used herein, the normoglycemic definition indicates HbA1c<5.7%, fasting glucose<100 mg/dL, 2-hr glucose on OGTT<140 mg/dL.

TABLE 20
Shifts in Glycemic Status from Normoglycemic
Baseline to Week 24 in Cohort 12

| | # Normoglycemic | # Normoglycemic | |
|---|---|---|---|
| | at Baseline | at Week 24 | Response |
| Placebo | 3 | 2 (1 became prediabetic) | 67% |
| CT-388 22 mg | 15 | 13 (2 had missing data) | 87% |

TABLE 21
Shifts in Glycemic Status from Prediabetic
Baseline to Week 24 in Cohort 12

| | # Prediabetes | # Normoglycemic | |
|---|---|---|---|
| | at Baseline | at Week 24 | Response |
| Placebo | 4 | 1 | 25% |
| CT-388 22 mg | 7 | 7 | 100% |

### Pharmacokinetics

Corresponding separations in the trough conc and in mean % WL curve at ˜week 8/9 suggests a strong exposure-response relationship between CT-388 concentration and magnitude of WL. CT-388 PK supports once-weekly dosing (t_{1/2}˜150 hrs) and is concordant with previous PK data from SAD & MAD cohorts. Pharmacokinetic results continue to support once-weekly dosing of CT-388.

### Safety and Tolerability

CT-388 was generally safe and well-tolerated up to 24 weeks of treatment with an AE profile consistent with the incretin drug class despite a rapid and steep titration. No SAEs or severe treatment related GI-related AEs were observed in CT-388 participants. Vitals, ECG, and safety laboratory findings were not clinically significant with patterns consistent with the incretin drug class.

### Summary and Conclusion

The Phase I results for CT-388 in people with obesity were positive. The study found that a once-weekly subcutaneous injection of CT-388 over 24 weeks resulted in significant weight loss in healthy adults with obesity compared to placebo. The weight loss achieved with CT-388 was clinically meaningful, with a mean placebo-adjusted weight loss of 18.8% (p-value<0.001). At week 24, 100% of CT-388 treated participants achieved a weight loss of >5%, 85% achieved >10%, 70% achieved >15%, and 45% achieved >20%. The treatment was well tolerated, with mild to moderate gastrointestinal-related adverse events being the most common, consistent with the incretin class of medicines that CT-388 belongs to. All participants with a pre-diabetes status at baseline became normoglycemic after 24 weeks of CT-388 treatment, whereas glycemic status of participants treated with placebo remained largely unchanged during this period.

Efficacy: Robust, statistically and clinically significant, and exposure-dependent weight loss effect (vs. placebo) is observed in 8 and 22 mg groups (p<0.001). At Week 12, placebo-adjusted % body weight change (95% CI) from baseline are −9.3 (−5.22,−13.40)% and −11.5 (−7.88,−15.11)%, respectively. The effect from CT-388 22 mg continues through the extension period of additional 12 weeks (Week 24): placebo-adjusted % body weight (95% CI) from baseline is −18.8 (−14.00,−23.6)%.

Over 24 weeks, a once-weekly subcutaneous injection of CT-388 achieved a clinically meaningful and statistically significant mean placebo-adjusted weight loss of 18.8% (p<0.001). At week 24, 100% of CT-388 treated participants achieved >5% weight loss, 70% achieved >15% and 45% achieved >20% weight loss.

Glucose Changes: In a subgroup with pre-diabetes at baseline, CT-388 treatment normalized glycemia in all patients, indicating its strong impact on glucose homeostasis. Placebo patients remained largely unchanged over 24 weeks. All seven participants with pre-diabetes at baseline became normoglycemic after 24 weeks of CT-388 treatment (100%); 1 out of 4 placebo-treated participants with prediabetes converted to normoglycemia (25%)

Safety: No new or unexpected safety signals were detected. Overall, CT-388 demonstrated a safety and tolerability profile consistent with its drug class. No SAEs or severe treatment related GI-related TEAE were observed in CT-388 participants. As expected, aggressive and steep up-titration resulted in higher frequency of GI-related TEAE in the up-titration phase. The incidence on GI-TEAEs had tendency to decrease during the Extension Period, when majority of patients were on stable dose.

### Conclusion

The results are highly encouraging for further development of CT-388 for both obesity and type 2 diabetes and underscore its potential to become a best-in-class therapy with durable weight loss and glucose control.

These findings suggest that CT-388 has the potential to improve weight-related comorbidities and to have a beneficial effect on glucose metabolism/control in people with and without T2D.

Example 4: Randomized, Double-Blind, Placebo-Controlled, Parallel Group, Multi-Center Phase 2 Study to Evaluate the Efficacy, Safety, and Tolerability of Once-Weekly CT-388 Administered for 48 Weeks to Participants with Obesity or Overweight with at Least One Weight-Related Comorbidity

This Example describes a multi-center, randomized, double-blind, placebo controlled, parallel group dose finding study to evaluate the efficacy and safety of CT-388 at low to high doses (from 4 mg to 24 mg).

The study will enroll approximately 450 participants with obesity class I and above (defined as BMI≥30 kg/m²) or with overweight (defined as BMI≥27 and <30 kg/m²) with at least one of the following weight-related comorbidities: prediabetes, hypertension, dyslipidemia, obstructive sleep apnea, or cardiovascular disease. Participants with any type of diabetes, obesity related to endocrinologic disease (e.g., Cushing syndrome), or use of specific medications (e.g., chronic glucocorticoids) will be excluded.

The study will evaluate CT-388 at 4, 8, 12, 16, and 24 mg as the target doses, which will be achieved via fixed dosing regimens (4 mg) or dose up-titration (8, 12, 16, and 24 mg). The treatment with CT-388 will be initiated at the starting dose of 2 or 4 mg. CT-388 will be administered QW for 48 weeks as an SC injection.

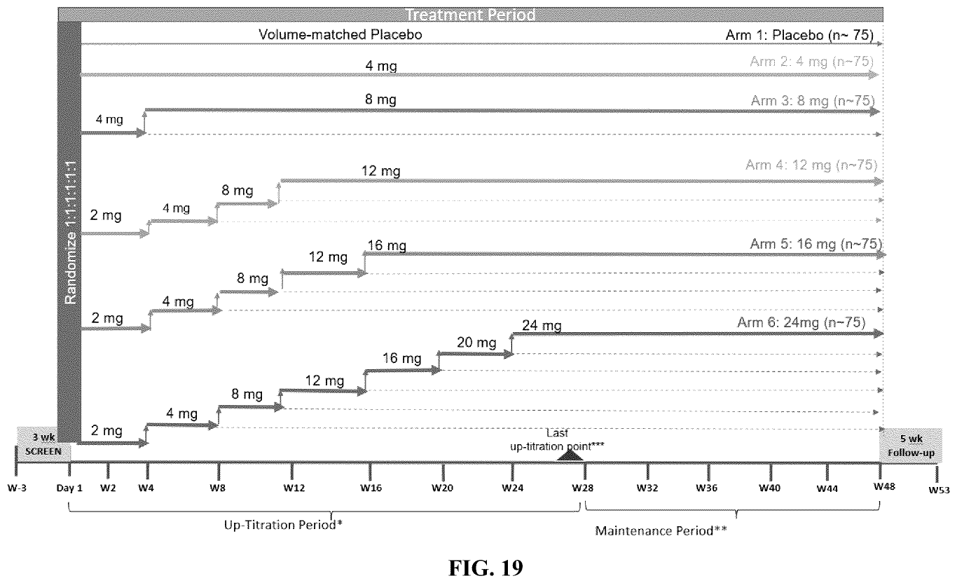

Eligible participants will be randomized to one of six treatment groups (CT 388 4, 8, 12, 16, or 24 mg or placebo) to enroll an equal number of participants (N=75/arm) across six arms (FIG. 19). Due to the titration scheme and study drug dose volume, this will be achieved via a two-step randomization, which results in the participants being randomized to volume-matched placebo and active treatment for each of the target dose levels in a ratio of 1:5. The randomization will be stratified by the participants' sex (male versus female). At each site, efforts should be made to enroll similar number of male and female participants. To avoid extreme imbalance of both sexes enrolled into the study, the number of enrolled female participants will be capped at 70%.

During a three-week screening period prior to treatment, participants will sign the informed consent form (ICF) and will be evaluated for their eligibility. After eligibility is confirmed, the participants will return for the day 1 visit and randomization will occur. As soon as the participant is randomized, they will be considered as enrolled in the study. The study drug dosing will be initiated on the same day after randomization.

The treatment period will consist of 48 weeks of treatment, which is divided into an Up-Titration Period (Day 1 to Week 28 pre-dose) and a Maintenance Period (Week 28 to Week 48). The overall dosing schema is presented in FIG. 19.

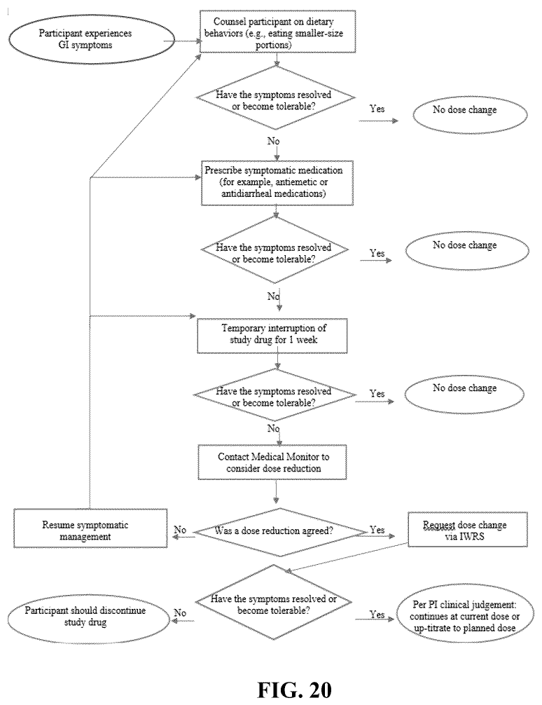

During the Up-Titration Period, the participants randomized to 8, 12, 16, or 24 mg will go through an up-titration phase when their dose will be up-titrated per the treatment schedule with the goal to up-titrate the dose on a monthly basis by four units (if applicable; except 2 mg to 4 mg) and achieve the assigned target dose by the end of the Up-Titration Period. If tolerability issues are observed, the algorithm noted in FIG. 20 should be followed. The last time point to reach the assigned target dose is Week 27.

The Maintenance Period will start on Week 28 and end on Week 48, with the last dose on Week 47, with the expectation that the assigned target dose will continue for 20 consecutive weeks. If a participant experiences tolerability issues, the algorithm noted in FIG. 20 will once again be followed.

Week 47 will be the last dose of the study drug. Week 48 will be the last treatment period visit. Participants will return to the clinic on Week 53 for the last safety follow-up visit.

During the study, all enrolled participants will receive dietary and physical activity counseling with a dietician or equivalently qualified delegate, ideally on a monthly basis and at least every 12 weeks during their study visits (or via phone call as appropriate) and re-enforced by the site staff on other study visits. Participants will be encouraged to maintain an approximately 500 kcal deficit per day relative to their estimated total energy expenditure (calculated once at randomization) and encouraged to undertake 150 minutes of physical activity per week such as walking. Participants will also be instructed to record their daily/weekly food intake, physical activity, and as applicable, menses, via study diaries, as well as smoking status/alcohol intake via TLFB calendars to evaluate their lifestyle patterns/regimen and consequently provide appropriate counseling.

### A. Objectives

The primary objective of this study is to evaluate the effect of low, medium, and high doses of CT-388 QW injection achieved via up-titration on percent change in body weight after 48 weeks of treatment compared with baseline.

The secondary objectives are:
to estimate the proportion of participants who achieved 5% weight loss after 48 weeks of treatment of CT-388 compared with baseline;
to assess the effect of CT-388 on:
   body weight-related parameters (body weight, BMI, waist-to-hip ratio, waist-to-height ratio, waist circumference, hip circumference, proportion of participants achieving specific weight and BMI thresholds); and
   glucose metabolism (glycated hemoglobin A1 [HbA1c], fasting plasma glucose, fasting insulin, C-peptide) and glycemic status.

The safety objective is to evaluate the safety and tolerability of CT-388 for 48 weeks versus placebo.

### B. Study Endpoints

The primary efficacy endpoint is percent (%) change in body weight from baseline to Week 48.

The secondary endpoints are:
a. body weight reduction≥5% from baseline to Week 48;
b. body weight reduction≥10% from baseline to Week 48;
c. body weight reduction≥15% from baseline to Week 48;
d. body weight reduction≥20% from baseline to Week 48;
e. body weight reduction≥25% from baseline to Week 48;
f. absolute change in body weight (kg) from baseline to Week 48;
g. percent (%) change in body weight from baseline to Week 48 by obesity class;
h. change from baseline to Week 48 in body weight-related parameters listed below:
   i. BMI (kg/m²);
   ii. waist circumference (cm), hip circumference (cm), and waist-to-hip ratio; and
   iii. waist-to-height ratio;
i. change from baseline in markers of glucose metabolism:
   i. HbA1c;
   ii. fasting plasma glucose;
   iii. fasting insulin;
   iv. fasting C-peptide;
   v. fasting HOMA-IR; and
   vi. quantitative Insulin Sensitivity Check Index (QUICKI); and
j. shift in glycemic status from baseline to Week 48 based on fasting plasma glucose and HbA1c.

The exploratory endpoints are:
a. change in cardiovascular risk factors such as:
   i. systolic and diastolic blood pressures (mm Hg);
   ii. triglycerides (mg/dL);
   iii. non-high-density lipoprotein (HDL) cholesterol (mg/dL);
   iv. HDL cholesterol (mg/dL);
   v. total cholesterol, low-density lipoprotein (LDL) cholesterol and very LDL cholesterol (mg/dL); and
   vi. NT-proBNP;
b. time (weeks) to achieve the final dose;
c. time (weeks) to achieve the weight loss plateau, which is defined as a weight change ≤5% over a 12-week interval and all subsequent weeks;
d. CT-388 PK and metabolite concentrations of CT-388, if applicable; and
e. treatment-emergent antidrug antibodies to CT-388 and cross reactivity to native GLP-1 and GIP.

The safety endpoints are:
a. number of participants with ≥1 TEAE, SAE, AE of special interest, or AE leading to study drug discontinuation;
b. TEAEs that occurred in >10% of enrolled participants;
c. number of participants with documented hypoglycemia (Level 1, 2, 3, per ADA 2024);
d. changes from baseline in the Columbia Suicide Severity Rating Scale (C-SSRS) and the Patient Health Questionnaire-9 (PHQ-9); and
e. change in safety laboratory values, vital signs, and electrocardiograms (ECGs).

### C. Rationale for Study Design

This study (CT-388-103) is designed to evaluate the efficacy and safety of CT-388 across different doses in adults living with obesity or overweight with at least one of the following weight-related comorbidities: prediabetes, hypertension, dyslipidemia, obstructive sleep apnea, cardiovascular disease, but excluding T2D. The study utilizes a randomized, double-blinded, parallel, placebo-controlled, multi-center design to rigorously assess efficacy and safety of SC QW administration of CT-388 versus placebo, in conjunction with a reduced-calorie diet and increased physical activity. A placebo arm is used as the comparator arm as the most rigorous test to evaluate treatment effect in complex patient population. To ensure all participants, including placebo participants, receive the standard of care for obesity management, the study design incorporates counseling on diet and lifestyle according to the local standards and per the guidance provided in the protocol throughout the study duration.

The duration of the study is 56 weeks, including a 3-week screening phase, a 48-week treatment period, and a 5-week safety follow-up phase (post-treatment). The primary endpoint for the study is defined as a percent change in body weight from baseline to Week 48. To adequately assess the objective to evaluate all doses of CT-388, including 24 mg, 48 weeks of treatment duration is required as that enables sufficient time for a gradual titration over a longer duration at each dose level before an up-titration (i.e., four weeks at a given dose). As a result, a 48-week treatment duration consists of two periods: Up-Titration Period (Day 1 to Week 28 predose) and Maintenance Period (Week 28 to Week 48).

During the up-titration phase, CT-388/placebo will be initiated at the doses of 2 or 4 mg with the subsequent planned dose increase to target levels (8, 12, 16, and 24 mg) scheduled at a minimum four week interval over the first 24 weeks of treatment. This cautious titration approach is expected to provide sufficient time for participants to adequately acclimatize to each dose. Nonetheless, to further maximize the number of participants who can reach the target randomized dose, the study allows the use of an additional four weeks (i.e., up to Week 27) to perform up-titration and achieve the maximal tolerated dose for each participant. During the Maintenance Period, while the expectation is that the participants will stay on their targeted/maximally tolerated dose until the end of the treatment period (i.e., for at least 20 weeks on the final dose), drug holiday or dose down-titration may be performed for safety or tolerability reason(s).

The follow-up phase, the 5-week off-treatment period, will provide safety follow-up and enable better understanding of changes in body weight and other relevant metabolic parameters following cessation of treatment.

Therefore, the study duration of 56 weeks, with 48 weeks of treatment period, is tailored to adequately evaluate the weight loss trajectory, durability of response and maintenance of weight loss over time, as well as to collect valuable data to characterize the longer-term safety and tolerability profile of CT-388. These findings will be instrumental in enabling a subsequent Phase 3 development program.

### D. Inclusion Criteria

Participants who meet all the following criteria prior to randomization may be enrolled to the study:
a. male or female, 18 years of age and up to 75 years of age at the time of providing informed consent
b. BMI≥30.0 kg/m² OR BMI≥27.0 and <30.0 kg/m² and previously diagnosed with at least one of the following weight-related comorbidities, such as:
   i. prediabetes: as defined by the 2024 ADA Standards of Medical Care in Diabetes
   ii. hypertension: defined as current use of blood pressure lowering agents initiated for hypertension, or with systolic blood pressure≥130 mm Hg or diastolic blood pressure≥80 mm Hg
   iii. dyslipidemia: defined as current use of lipid-lowering agents initiated for dyslipidemia, or with low-density lipoprotein (LDL)≥160 mg/dL (4.1 mmol/L) or triglycerides≥150 mg/dL (1.7 mmol/L), or high-density lipoprotein (HDL)<40 mg/dL (1.0 mmol/L) for men or HDL<50 mg/dL (1.3 mmol/L) for women, based on central laboratory value
   iv. previously diagnosed obstructive sleep apnea, or
   v. previously diagnosed cardiovascular disease (for example, ischemic cardiovascular disease, New York Heart Association (NYHA) Functional Class I-II heart failure)
c. History of ≥1 self-reported unsuccessful diet/exercise effort to lose body weight.

### E. Exclusion Criteria

Participants who meet any of the following criteria prior to treatment will not be enrolled into the study.

### Diabetes Related

a. Have a prior history/diagnosis of any type of diabetes mellitus (e.g., Type 1, Type 2, gestational), or a history of ketoacidosis or hyperosmolar state.
b. Have laboratory evidence of diabetes mellitus determined based on fasting glucose or HbA1c during the screening period.
Note: diagnosis of prediabetes (determined by fasting glucose or HbA1c is not exclusionary, but participants must not be on any type of glucose-lowering treatments (pharmacologic or dietary supplements), including but not limited to metformin, berberine, etc.

### Obesity Related

c. Have had any of these procedures—LAP-BAND®, intragastric balloon, duodenal sleeve, resurfacing, liposuction, and/or abdominoplasty—<1 year before the screening visit or planned during study participation.

### Other Medical Related

d. Have a known clinically significant or active gastric emptying abnormality (e.g., severe gastroparesis or gastric outlet obstruction, intestinal obstruction, or any GI motility disorders); malabsorption, including chronic constipation/diarrhea, celiac disease, inflammatory bowel disease, or bowel resection; or chronically take drugs that directly affect GI motility (e.g., anticholinergics, 5-hydroxytryptamine [serotonin] antagonists, opiates).
e. Have a history or presence of chronic pancreatitis, or presence of acute pancreatitis within 6 months before screening.
f. Have a history of complications from gall stones and/or acute cholecystitis within 6 months of screening (prior history of cholecystectomy is not exclusionary).
g. Have uncontrolled hypertension (systolic blood pressure≥160 mm Hg and/or diastolic blood pressure≥100 mm Hg). Participants on antihypertensive medications must be on a stable dose for ≥2 months before the screening visit.
h. Have any of the following cardiovascular conditions within 6 months before screening: hospitalization due to congestive heart failure, myocardial infarction, cerebrovascular accident (stroke), transient ischemic attack, unstable angina, coronary artery bypass graft, percutaneous coronary intervention (including those who may have percutaneous transluminal coronary angioplasty [PTCA] planned and/or anticipated during study participation, such as those with prior angiographic evidence that may require PTCA).
i. Have NYHA Functional Classification III or IV congestive heart failure.
j. A personal or family history of long QT syndrome, family history of sudden death in a first-degree relative (parents, sibling, or children) before the age of 40 years, or a personal history of unexplained syncope within the last year.
k. Have uncontrolled thyroid disease, defined as active symptoms (e.g., palpitations, lethargy, weight gain/loss) and/or having a thyroid-stimulating hormone (TSH) level outside the normal reference range of the central laboratory at the screening visit.
   Note: Participants receiving adequate treatment for hypothyroidism may be included, provided they are clinically asymptomatic, and their thyroid hormone replacement dose has remained stable for ≥2 months before the screening visit.
l. Have obesity induced by other endocrinologic disorders (e.g., Cushing syndrome, acromegaly, inadequately treated hypothyroidism) or diagnosed monogenetic or syndromic forms of obesity (e.g., Melanocortin 4 Receptor deficiency or Prader-Willi syndrome).
m. Have a history/diagnosis of major depressive disorder within 2 years of the screening visit, or any history/diagnosis of other severe psychiatric conditions (e.g., schizophrenia, bipolar disorder, eating disorder, other serious mood, anxiety, or hyperactivity disorder).
   Note: Prospective participants with depression or anxiety whose disease state, in the opinion of the Investigator, is considered stable and expected to remain stable throughout the course of the study, may be considered for inclusion only if they are adequately controlled by stable doses of selective serotonin reuptake inhibitors (other than paroxetine) for ≥6 months before the screening visit, and expected to continue at the same dose for the entire study duration. Participants maintained on other types of anti-depression/mood stabilizers are excluded from this study as many are associated with body weight changes (see Section 7.3.2 for a more detailed list of prohibited medications).
n. Have a PHQ-9 score of ≥15 at the screening visit and Day 1.
o. Have any lifetime history of a suicidal attempt, or suicidal behavior within 30 days before the screening visit.
p. Suicidal ideation on the C-SSRS assessed at the screening visit or Day 1 and determined as:
   vii. A “yes” to either Question 4 (Active Suicidal Ideation with Some Intent to Act, Without Specific Plan) OR
   viii. A “yes” answer to Question 5 (Active Suicidal Ideation with Specific Plan and Intent) on the “Suicidal Ideation” portion of C-SSRS OR
   ix. A “yes” answer to any of the suicidal-related behaviors (Actual Attempt, Interrupted Attempt, Aborted Attempt, Preparatory Act or Behavior) on the “Suicidal Behavior” portion of the C-SSRS AND the ideation or behavior occurred within the past month.
q. Have history of any hematologic conditions that may interfere with HbA1c measurement (e.g., hemolytic anemias, sickle cell disease, other hemoglobinopathies).
r. Have a family or personal history of medullary thyroid carcinoma or a genetic condition that predisposes to medullary thyroid carcinoma (i.e., thyroid C-cell hyperplasia, or multiple endocrine neoplasia Type 2A or Type 2B).
s. ECG findings at the screening visit and repeat at Day 1:
   x. Indicative of active cardiac disease or clinically significant variants/abnormalities as determined by the Investigator that may interfere with the interpretation of ECG changes over the study duration;
   xi. QTc (Fridericia) interval ≥450 msec for men and ≥470 msec for women;
   xii. Resting heart rate<45 bpm or >100 bpm; or
   xiii. Any other pathologic heart rhythms.
t. Any of the following findings present during the screening period, as determined by the central laboratory:
   xiv. Alanine aminotransferase (ALT), or aspartate aminotransferase (AST), or gamma glutamyl transferase (GGT)>3.0× the upper limit of normal (ULN) for the reference range
   xv. Alkaline phosphatase (ALP)>1.5×ULN for the reference range
   xvi. Total bilirubin>1×ULN for the reference range
   xvii. Amylase or lipase>2×ULN for the reference range
   xviii. Calcitonin≥20 ng/L
   xix. Estimated glomerular filtration rate (eGFR)<60 mL/min/1.73 m² by the 2021 CKD-EPI creatinine equation
   xx. Fasting plasma triglycerides≥500 mg/dL
   xxi. Acute or chronic viral hepatitis (determined based on hepatitis B surface antigen [HbsAg] or presence of HCV RNA)
   xxii. Positive HIV antibody screen
   xxiii. Hemoglobin value <11 g/dL (men) or <10 g/dL (women)
   xxiv. White blood cells (WBC)<the lower limit of normal (LLN) for the reference range
   xxv. Platelets<LLN for the reference range.

### Prior/Concomitant Medications

u. Are receiving or have received within three months before the screening visit chronic (>14 consecutive days) systemic glucocorticoid therapy (excluding topical, intraocular, intranasal, intraarticular, or inhaled preparations) or have evidence of a significant, active autoimmune abnormality (e.g., lupus or rheumatoid arthritis) that has required (within the last 3 months) or is likely to require, in the opinion of the Investigator, concurrent treatment with systemic glucocorticoids (excluding topical, intraocular, intranasal, intra-articular or inhaled preparations) during study participation.

### F. Diet and Physical Activity Counseling

All enrolled participants in the study will receive counseling with a dietician, or equivalently qualified delegate, according to local standards, to receive lifestyle management counseling ideally on a monthly basis and at least every 12 weeks during the study visits (or via phone call as appropriate) on day 1, and weeks 12, 24, 36, and 48. Diet and exercise goals established during the lifestyle consultation and the importance of adherence to the lifestyle component of the trial will be reinforced at each trial contact by study staff. Participants will be instructed on how to capture their physical activity and food intake in study-provided diaries to facilitate review and will be provided appropriate guidance during their study visits.
a. Dietary counseling will consist of advice on healthy food choices and focus on calorie restriction using a hypocaloric diet with macronutrient composition of:
   xxvi. Maximum 25% of energy from fat
   xxvii. ˜25% of energy from protein
   xxviii. ˜50% of energy from carbohydrates.
b. An energy deficit of ˜500 kcal/day compared with the participant's estimated total energy expenditure is recommended.
   xxix. Total energy expenditure (TEE) will be calculated using Body Weight Planner per National Institute of Diabetes and Digestive and Kidney disease (available via the NIH), which will take into account participant's weight and level of physical activity.
c. Participants with significant suppression of appetite must be counselled to consume at least 900 kcal/day, with the emphasis on an adequate protein intake (meal replacements such as with high protein Ensure/Boost may also be offered).
d. Regular physical activity (150 minutes of physical activity per week, e.g., walking) is encouraged.
e. The hypocaloric diet should be continued after randomization and throughout the treatment period.
f. If a BMI≤22 kg/m² is reached, the recommended energy intake should be recalculated with no kcal deficit for the remainder of the trial.

### G. Definitions of Glycemic Status

Participants in this study will be evaluated for their glycemic status and categorized into those with normoglycemia, prediabetes, or diabetes as defined by the ADA Standards of Care (ADA 2024).

The initial glycemic assessment, to determine the status of normoglycemia, prediabetes, or overt diabetes, will be conducted during the screening phase. This assessment will utilize measurements of fasting plasma glucose and HbA1c. Participants will be excluded if the screening results indicate a definitive diagnosis of diabetes, characterized by fasting plasma glucose levels of ≥126 mg/dL (7.0 mmol/L) or HbA1c values ≥6.5% (48 mmol/mol)).

Only participants with normoglycemia or prediabetes will be enrolled into the study. Should any participant be diagnosed with T2D during the screening process, they will be directed to consult with their primary care physician for appropriate management.

Throughout the 48-week treatment phase, the study will maintain regular monitoring of glycemic status. This monitoring will be facilitated through periodic checks of fasting plasma glucose or HbA1c levels.

TABLE 22
Criteria for the Screening and Diagnosis of Prediabetes
and Diabetes (Glycemic Status Definitions)

| Parameter | Normoglycemia | Prediabetes | Diabetes |
|---|---|---|---|
| HbA1C | <5.7% | 5.7-6.4% | ≥6.5% |
| | (<39 mmol/mol) | (39-47 mmol/mol) | (48 mmol/mol)^{b} |
| FPG | <100 mg/dL | 100-125 mg/dL | ≥126 mg/dL |
| | (<5.6 mmol/L) | (5.6-6.9 mmol/L)^{a} | (7.0 mmol/L)^{b} |
| Random | — | — | ≥200 mg/dL |
| plasma | | | (11.1 mmol/L)^{c} |
| glucose | | | |

^{a}For all these tests, risk is continuous, extending below the lower limit of the range and becoming disproportionately greater at the higher end of the range.
^{b}In the absence of unequivocal hyperglycemia, diagnosis requires 2 abnormal test results from the same sample or in 2 separate samples.
^{c}Only diagnostic in a patient with classic symptoms of hyperglycemia or hyperglycemic crisis.
Adapted from Tables 2.2 and 2.5 in the complete 2024 Standards of Care (ADA 2024).

### H. Clinical Evaluations

### Weight and BMI

Body weight measurements should be done in a consistent manner using a calibrated electronic scale capable of measuring weight in kilograms (kg) to one decimal place.

All weights for a given participant should be measured using the same scale, whenever possible, at approximately the same time in the morning after evacuation of bladder contents.

Body weight must be measured in fasting state. If the participant is not fasting, the participant should be called in for a new visit within the allowable visit window to have the fasting body weight measured.

### Waist and Hip Circumference

Waist circumference should be measured in the horizontal plane and at the midpoint between the lower margin of the last palpable rib and the top of the iliac crest.
a. Measurements should be taken at the end of a normal expiration using a non-stretchable measuring tape. The tape should lie flat against the skin without compressing the soft tissue.
b. The waist circumference should be measured twice, rounded to the nearest 0.5 cm. The measuring tape should be removed between the 2 measurements. Both measurements will be recorded in the eCRF. If the difference between the 2 measurements exceeds 1 cm, this set of measurements should be discarded and the 2 measurements repeated.

### Vital Signs

Vital signs (temperature, blood pressure, pulse, respiratory rate) will be obtained at each study visit. Vital signs will be conducted after the participant has rested for ≥25 minutes in a seated, supine, or semi-recumbent position. For each parameter, 3 measurements will be taken using the same arm, preferably the nondominant arm and the recordings should be taken ≥1 minute apart. If possible, blood pressure must be taken with an automated blood pressure instrument. If blood pressure and pulse measurements are taken separately, pulse should be taken before blood pressure. Remind the participant to avoid caffeine, smoking, and physical activity within 1 hour before the measurement of vital signs at all visits to the study site/clinic.

### PK Blood Sample

CT-388 plasma concentration will be analyzed using predose samples collected in all enrolled study participants to determine the trough CT-388 levels throughout the study drug administration period.

In addition, a subset of participants (i.e., those willing to provide informed consent and able to return to the study site for multiple blood draws over a week), will undergo additional PK sampling for population PK post-dose assessment during the 48-week treatment period. These participants can choose from 3 distinct post-dose PK sampling time points, 24 to 48 hours (i.e., 1 to 2 days) after dosing, 48 to 72 hours (i.e., 2 to 3 days) after dosing, or 72 to 96 hours (i.e., 3 to 4 days) after dosing for each of the following 3 periods without repeats:
1) Week 1 to Week 12;
2) Week 13 to Week 28; and
3) Week 29 to Week 48.

Participants must choose a different time point for each of the 3 periods. This approach ensures that a diverse range of PK data is collected, enabling a more comprehensive understanding of the PK variability among different individuals and over time.

Samples from participants receiving placebo will not be assessed in the first instance but will be retained for subsequent analysis if appropriate. Samples will not be analyzed in real time but will be batched for analysis throughout the study.

Plasma concentration obtained at these time points will be used for population PK, PD, and safety analyses.

Metabolites of CT-388 may be measured by a specific validated liquid chromatography-tandem mass spectrometry (LC-MS/MS) assay, or other fit for purpose methods as appropriate. Sufficient samples should be stored for the possibility that CT-388 metabolites may be measured from these samples during the study when assay methods are available. Unused sample material may be used for the purpose of current CT-388 assay improvement, and for the assessment of exploratory plasma biomarkers.

Any leftover PK samples remaining after the specified analyses may also be used for additional assay development/validation experiments (e.g., metabolite identification, ADA assay validation or exploratory plasma biomarkers).

### Dose Titration

Dosage for each participant in CT-388 low- to high-dose treatments will be escalated as shown in FIG. 19. Dose escalations may occur at, or after, but not before, the designated time for up-titration.

The last up-titration/re-challenge time point is Week 27. FIG. 20 should be followed to help participants stay with the final dose during the Maintenance Period (Week 28 to the last dose of study drug at Week 47).

Participants who experience GI-related symptoms should be managed per FIG. 20.

Participants randomized to CT-388 Low to High Dose treatments would need to tolerate a minimum of 0.2 mL to continue on study drug. If the Investigator believes that the participant will not tolerate 0.2 mL despite providing additional time to adjust to that dose level and undertaking all the symptomatic-relief measures to mitigate tolerability issues, then that individual would discontinue administration of their assigned study drug.

### Adverse Events

An AE is any unfavorable and unintended sign (including an abnormal laboratory finding), symptom, or disease temporally associated with the use of a medical treatment or procedure that may or may not be considered related to the medical treatment or procedure. TEAEs are defined as AEs that worsen or commence on or after first study drug administration.

All AEs regardless of severity or how identified (e.g., volunteered, elicited, noted on physical examination) will be recorded throughout the study (i.e., from screening until that last follow up visit or 45 days after the last dose of study drug, whichever is later).

Participants will be followed for resolution of AEs, by querying the participants for an ongoing AE until the earlier of AE resolution or the last study visit.

The Investigator will rate the severity/intensity of each AE. Investigators may reference the National Cancer Institute-Common Terminology Criteria for Adverse Events (CTCAE), Version 5.0. The CTCAE displays Grades 1 through 5 with unique clinical descriptions of severity for each AE. A general grading (severity/intensity) scale, for those events not listed specifically, is provided at the beginning of the CTCAE reference document.

### I. Data Analysis and Statistical Considerations

A separate Statistical Analysis Plan (SAP) will be prepared once the protocol is approved by regulatory authority and before database lock (unblinding). This document will provide further details regarding the definition of analysis variables and analysis methodology to address all study objectives. The SAP will serve as a complement to the protocol and supersedes it in case of differences.

The statistical evaluation will be performed using SAS® software version 9.4 or higher (SAS Institute, Cary, NC). All data will be listed, and summary tables will be provided. Summary statistics will be presented by treatment group. Continuous variables will be summarized for the measured values, change and percent change from baseline values using the number of observations, mean, standard deviation, median, minimum, and maximum. Categorical variables will be summarized with counts and percent for each category. Treatment differences will be presented together with 95% confidence intervals as appropriate, unless specified otherwise.

P-values will be presented with 3 decimal points. Values below 0.001 will be presented as ‘<0.001’. Statistical significance will be concluded after comparing p-values with significance levels [0.035 (or 0.05), 0.01 or 0.001]. The comparison will be performed without rounding.

### Sample Size

For the percent change from baseline to Week 48 in body weight, assuming the standard deviation to be 10%, 60 completers per treatment arm will give ≥91% power to detect a true difference of at least 6.4% between a CT-388 dose and placebo. This assumes a 2-sided test at the 3.5% significance level with a 2-sample t-test. The overall Type 1 error rate for this study for the primary efficacy endpoint with 5 dose groups compared with placebo will be approximately 13%.

In total, approximately 450 participants will be randomized equally to receive placebo, 4, 8, 12, 16, and 24 mg CT-388 achieved via fixed dosing regimen and up-titrations for 48 weeks of treatment so that there will be 75 participants in each treatment group. Assuming a dropout rate of up to 20% during the treatment period, this will ensure ≥360 participants (i.e., ≥60 per treatment arm) to complete the 48-week treatment period.

### Randomization

The goal of the randomization is to achieve an equal number of participants (N=75/arm) across 6 arms: placebo, CT-388 4, 8, 12, 16, and 24 mg. Due to the titration scheme and study drug dose volume, this will be achieved via a 2-step randomization that leads to participants being randomized to volume-matched placebo and active treatment for each of the target dose levels in 1:5 ratio:
Step 1. Randomize the participants in a ratio of 1:1:1:1:1 to 5 target dose levels (CT-388 4, 8, 12, 16 or 24 mg). All participants in the same target dose level (placebo or active) will receive the study drug with the same volume in each injection throughout the study for the planned dose level. This step will be blinded so that the participants and the study staff will not know the target dose level assignment.
Step 2. In each target dose level, randomize the participants in a 1:5 ratio to placebo and active. This step will be blinded so that the participants and Investigator will not know whether the participant is on placebo or active treatment.

With this randomization, staff and participants will not know at randomization to which dose group participants are assigned and they will not know whether they are receiving CT 388 or placebo. However, the site and the site staff will know the volume of the study drug to be administered at each visit. The randomization will be performed by IRT.

Randomization will be stratified by sex (male and female). In addition, to avoid extreme imbalance of both sexes enrolled into the study, the number of enrolled female participants will be capped at 70%.

For all summaries and analysis by treatment arms, placebo participants will be pooled together to form the placebo group.

### Analysis Sets

The analysis sets are detailed in the following sections. A summary of analysis sets will be provided. Individual analysis set flags will also be listed.
a. Full Analysis Set: All randomized participants will be included in the Full Analysis Set (FAS) based on the randomized treatment assigned. All baseline characteristics and demographic data will be summarized using the FAS. The FAS will also be used for all efficacy and PD analysis.
b. Safety Set: All randomized participants who receive ≥1 dose of study drug will be included in the Safety Set based on actual treatment received. All safety endpoints will be summarized using the safety population.
c. PK Set: All randomized participants who receive ≥1 dose of CT-388 and have ≥1 valid plasma CT-388 concentration value will be included in the PK Set. Population PK analyses will be conducted using the PK Set.

Example 5: A Randomized, Double-Blind, Placebo-Controlled, Parallel Group, Multi-Center Phase 2 Study to Evaluate the Efficacy, Safety, and Tolerability of Once-Weekly CT-388 Administered Subcutaneously for 48 Weeks to Participants Who are Overweight or Obese with Type 2 Diabetes Mellitus

This Example describes a multi-center, randomized, double-blind, placebo-controlled, parallel-group study to evaluate the efficacy and safety of CT-388 from low to high doses (4, 8, 16, and 24 mg) in obese or overweight participants with T2DM.

Approximately 360 participants who are overweight (defined as BMI≥25 and <30 kg/m²) or obese (defined as BMI≥30 kg/m²) and have T2DM with inadequate glycemic control (defined as HbA1c≥7.0% and ≤10.5%) will be enrolled. Participants must be diagnosed with T2DM for at least six months prior to treatment and have T2DM managed with diet and exercise alone or treated with metformin or a sodium-glucose cotransporter 2 (SGLT-2) inhibitor as monotherapy or in combination. Antihyperglycemic medications must be on the stable dose for at least three months prior to treatment.

Participants with other diseases associated with increased weight or increased glucose levels (e.g., Cushing syndrome, type 1 diabetes), are excluded.

CT-388 will be administered QW as a subcutaneous (SC) injection. CT-388 will be evaluated at final target doses of 4, 8, 16, and 24 mg achieved via dose up titration for a total treatment duration of 48 weeks. Treatment with CT-388 will be initiated at a starting dose of 2 mg or 4 mg. In addition, evaluation of the potential efficacy of CT-388 on glycemic control with low doses (2 mg and 4 mg) over 12 weeks of treatment will be performed.

Eligible participants will be randomized to one of five treatment groups (CT-388 4, 8, 16, 24 mg, or placebo) to enroll an equal number of participants (N=72/group). Due to the titration scheme and study drug dose volume, this will be achieved via a two-step randomization, which results in the participants being randomized to volume-matched placebo and active treatment for each target dose level in a ratio of 1:4, stratified by participant's sex (male versus female), HbA1c value at screening (≤8.5% or >8.5%), and by BMI<35 or ≥35. At each site, efforts should be made to enroll a similar number of male and female participants, to avoid an extreme imbalance between sexes in the study.

The Screening Period may be ≤21 days, but not longer. Participants will be instructed to initiate the recording of data in their diaries to collect information on their food intake, exercise, glucose levels, and menses if applicable. These data will be collected via mobile application or paper diary and will support evaluation of potential safety concerns (e.g., hyperglycemic or hyperglycemic events), discussion related to type 2 diabetes education, and life-style management. This data collection will continue throughout the study participation.

Following the Screening Period, eligible participants will enter a two-week Lead-in Period to confirm their eligibility, perform their MRI, where applicable, and initiate monitoring of their glycemic stability.

Starting at the Lead-In period and over the study duration, all eligible participants will self-monitor their fasting blood glucose levels, at a minimum of three times per week, and as needed based on symptoms; using the study-specific glucometer and supplies provided to each participant.

Participants who provide consent will have their baseline MRI performed.

Following the two-week Lead-in Period, the participants will return for the Day 1 visit. Once eligibility is confirmed, the participant will be randomized. At this point the participant will be considered enrolled into the study and will enter the Treatment Period. Following randomization and pre-dose study procedures, study drug administration will be initiated. The Treatment Period is 48 weeks in duration, divided into an Up-Titration Period (Day 1 to Week 28 Pre-dose) and a Maintenance Period (Week 28 to Week 48).

The study drug will be administered subcutaneously once a week. Participants are not allowed to self-administer the study drug and are required to return to the clinic for drug administration. If clinic visits are not feasible, home-nurse visits may be arranged. Ideally, the dosing will occur on the same day and time of the week through the entire study.

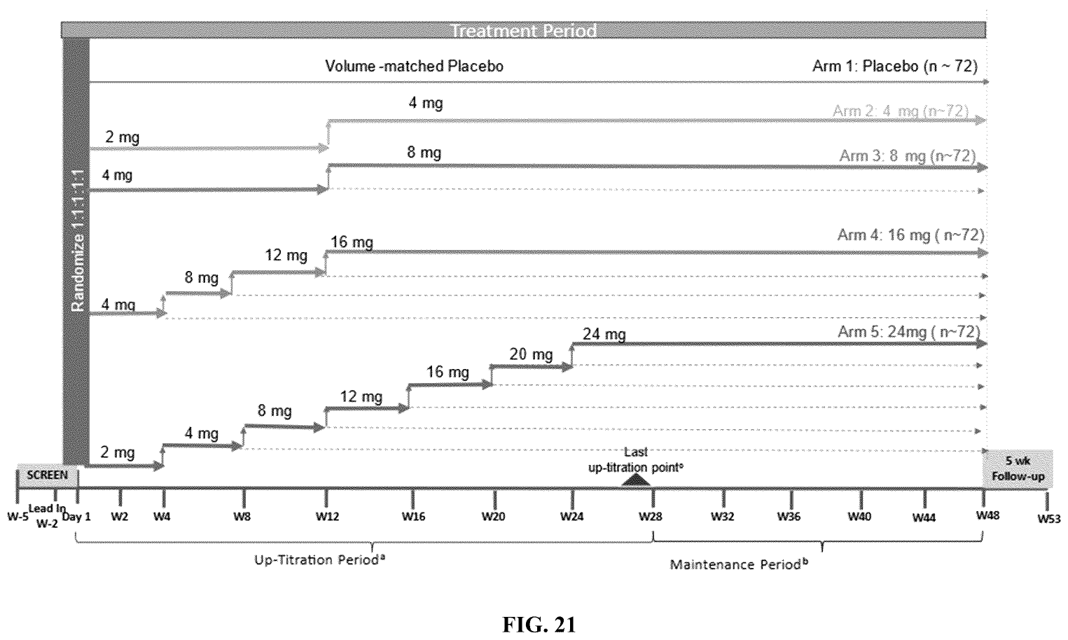

During the Up-Titration Period, the participants will have their dose up-titrated (FIG. 21), with the goal of up-titrating the dose per the protocol specified schedule and achieving the assigned target dose by the end of the Up Titration Period. Participants assigned to low target dose treatment groups (4 mg and 8 mg) will initiate CT-388 treatment with 2 mg or 4 mg, respectively, with the dose up titration performed after the initial 12 weeks of treatment. Participants assigned to middle (16 mg) and high (24 mg) doses of CT-388 will initiate their treatment with 2 mg for the initial 4 weeks, and will perform up-titration to 4 mg, and thereafter by up-titrating the dose by 4 mg monthly up to the assigned dose. If tolerability issues are observed, the algorithm noted in FIG. 20 should be followed. The last time point when participant can reach the assigned target dose is Week 27.

The Maintenance Period starts on Week 28 and ends on Week 48, with the last dose on Week 47. The expectation during the Maintenance Period is that the achieved dose will continue for 20 consecutive weeks. If a participant experiences tolerability issues, the algorithm noted in FIG. 20 should be followed. If a participant experiences significant weight loss (BMI≤22 kg/m²), caloric deficit should be discontinued.

In addition to weekly self-monitoring of fasting glucose, participants will perform a 7-point self-monitoring blood glucose (SMBG) assessment five times during the study: during the Lead-In Period (Day −14 to Day 1 pre-dose), at Week 12, and at the beginning and end of the Maintenance Period (Week 28 to 29 and Week 47 to 48). If, based on glucose self-monitoring or SMBG, hypoglycemia or hyperglycemia is detected, the participant must notify the site so that appropriate follow up is performed. Participants with severe persistent hyperglycemia may initiate rescue therapy. During the study all participants will be monitored for safety or tolerability issues, including but not limited to hyperglycemia, hypoglycemia, tachycardia, and liver and renal impairment. If safety or tolerability issues are observed, dose modification may be performed, and if any of the discontinuation criteria are met, the participant will be discontinued from study drug.

Blood samples for population PK analysis will be collected from selected participants at specific scheduled visits.

Participants enrolled into the MRI-selected sites will undergo a full body MRI to evaluate body composition, visceral fat, and brain perfusion. MRI will be performed twice during the study: during the Lead-In Period and at the end of the Treatment Period. Week 47 will be the last dose of the study drug. Week 48 will be the last Treatment Period visit; participants will return to the clinic at Week 53 for the Safety Follow-up Visit.

During the study, all enrolled participants will receive diet and exercise counseling with a dietician or equivalently qualified delegate. Consultation will be provided based on participant's data collected via mobile application or a paper diary and will be performed ideally monthly and at least every 12 weeks during their study visits, or via phone call as appropriate. Participants will be encouraged to maintain a T2DM appropriate diet and maintain a caloric deficit of approximately 500 kcal per day relative to their estimated total energy expenditure and encouraged to undertake 150 minutes of physical activity per week.

To adequately assess the objective to evaluate all doses of CT-388, including the 24 mg dose, 48 weeks of treatment is required to enable sufficient time for a gradual titration over a longer duration at each dose level before an up-titration (i.e., at least 4 weeks at a given dose). As a result, the 48-week treatment duration consists of two periods: the Up-Titration Period (Day 1 to Week 28) and the Maintenance Period (Week 28 to Week 48).

During the up-titration phase, CT-388 or placebo will start at doses of 2 or 4 mg with the subsequent planned dose increase to target levels (4, 8, 16, and 24 mg) scheduled at a minimum 4 week interval over the first 24 weeks of treatment. This cautious titration approach is expected to provide sufficient time for participants to adequately acclimatize to each dose. Nonetheless, to maximize the number of participants who can reach the target randomized dose, the study design allows for the use of an additional four weeks (i.e., up to Week 27) to perform up-titration and achieve the maximal tolerated dose for each participant. A drug holiday or dose down-titration may be performed for safety or tolerability reason(s).

In addition, the first 12 weeks of treatment will be utilized to evaluate the effect of low doses of CT-388 (2 mg and 4 mg) in participants with T2DM and to support identifying minimal efficacious dose on markers of glucose metabolism and the effect of slow up-titration on the body weight trajectory.

During the Maintenance Period, while the expectation is that the participants will stay on their targeted/maximally tolerated dose until the end of the treatment period (i.e., for at least 20 weeks on the final dose), a drug holiday or dose down-titration may be performed for safety or tolerability reason(s).

The Follow-up Period, the five-week off-treatment period, will provide safety follow-up and enable a better understanding of changes in body weight and other relevant metabolic parameters following cessation of treatment.

Therefore, the study duration of 57 weeks, with 48 weeks of the Treatment Period, is tailored to adequately evaluate the weight loss trajectory and effect on HbA1c; durability of response; maintenance of weight loss and HbA1c control over time; as well as to collect valuable data to characterize the longer-term safety and tolerability profile of CT-388 in participants with T2DM. These findings will be instrumental in enabling a subsequent Phase 3 development program.

### Inclusion Criteria

Participants who meet all the following criteria prior to randomization may be enrolled into the study.
a. Are male or female, between 18 and 75 years of age at the time of providing signed informed consent.
b. BMI≥25.0 kg/m² at the Screening and Day 1 Visits
c. Have a diagnosis of T2DM according to the World Health Organization classification or other locally applicable standards for at least 6 months before Screening
d. Have an HbA1c≥7% and ≤10.5% at the Screening and Lead-In Visit
e. Management of T2DM with diet and exercise alone or T2DM managed with diet and exercise alone or treated with metformin or a sodium-glucose cotransporter 2 (SGLT-2) inhibitor as monotherapy or in combination, per approved local label. Participants on metformin or SGLT-2 inhibitors must be on a stable therapy for >3 months prior to the Screening Visit
f. History of ≥1 self-reported unsuccessful diet/exercise effort to lose body weight.
g. Female participants who are women of childbearing potential (WOCBP) must agree to remain abstinent (refrain from heterosexual intercourse) or use contraceptive methods with a failure rate of <1% per year during the treatment period and for 45 days after the final dose of CT-388.

### Exclusion Criteria

Participants who meet any of the following criteria prior to randomization will not be enrolled into the study.
a. Have type 1 diabetes mellitus (T1DM), history of ketosis or hyperosmolar state/coma, or any other types of diabetes except T2DM.
b. Have had one or more episodes of Level 3 hypoglycemia or has hypoglycemia unawareness within the six months prior to Screening.
c. Have at least two confirmed fasting self-monitored blood glucose values >270 mg/dL on two nonconsecutive days between screening and randomization.
d. History or presence of proliferative diabetic retinopathy, diabetic macular edema, non-proliferative diabetic retinopathy that requires acute treatment based on a dilated fundoscopic examination performed by an ophthalmologist or optometrist between screening and randomization.
e. Evidence of clinically significant/active nephropathy, neuropathy (including resting tachycardia, orthostatic hypotension, diabetic diarrhea).
f. Treatment with any other antihyperglycemic medication except metformin or SGLT-2, including but not limited to dipeptidyl peptidase 4 (DPP4) inhibitors, glucagon-like peptide-1 (GLP-1) or GLP-1/glucagon-like peptide-1 receptor agonist (GIP-RA), sulfonylurea, amylin analog.
g. Self-reported body weight change of >5 kg within three months of the Screening Visit.
h. Any unbalanced/extreme diets such as very low calorie, low carbohydrate, very high protein, ketogenic or intermittent diets, within three months of the Screening Visit, or plan to be on such diets during the study.
i. Current participation, or within the last three months before Screening, in an organized weight reduction program
j. Have a prior or planned surgical treatment or procedure (of any type) for obesity (excluding liposuction or abdominoplasty if performed >one year prior to Screening).
k. Treatment with any medication, device, dietary supplements, or any over-the-counter preparations (approved or investigational) that promote weight loss within six months of the Screening Visit, or plan to be treated during the study participation.
l. Have a known clinically significant or active gastric emptying abnormality (e.g., severe gastroparesis or gastric outlet obstruction, intestinal obstruction, or any gastrointestinal [GI] motility disorders), malabsorption, including chronic constipation/diarrhea, Celiac disease, inflammatory bowel disease, bowel resection, or chronically take drugs that directly affect GI motility (e.g., anticholinergics, 5 hydroxytryptamine 3 antagonists, opiates).
m. History or presence of chronic pancreatitis, or presence of acute pancreatitis within six months before screening.
n. Have a history of complications from gall stones and/or acute cholecystitis within six months of Screening (prior history of cholecystectomy is not exclusionary). NOTE: Participants who have had a procedure to remove gallstones and/or the gallbladder with no long-term complications are eligible as long as the procedure was completed at least three months prior to screening.
o. Have uncontrolled hypertension (systolic blood pressure≥160 mm Hg and/or diastolic blood pressure≥100 mmHg). Participants on antihypertensive medications must be on a stable dose for ≥one month before the Screening Visit.
p. Have an elevated resting pulse rate (>100 bpm) at the Screening Visit, and Day 1
q. Have any of the following cardiovascular conditions within six months before screening: hospitalization due to congestive heart failure, myocardial infarction, cerebrovascular accident (stroke), transient ischemic attack, unstable angina, coronary artery bypass graft, percutaneous coronary intervention (including those who may have percutaneous transluminal coronary angioplasty [PTCA] planned and/or anticipated during study participation, such as those with prior angiographic evidence that may require PTCA).
r. Have New York Heart Association Functional Classification III or IV congestive heart failure.
s. Have a personal or family history of long QT syndrome, family history of sudden death in a first-degree relative (parents, sibling, or children) before the age of 40 years, or a personal history of unexplained syncope within the last year.
t. Have uncontrolled thyroid disease, defined as active symptoms (e.g., palpitations, weight gain/loss) and/or having a thyroid-stimulating hormone value outside the normal reference range of the central laboratory at the Screening Visit. NOTE: Participants receiving adequate treatment with thyroid hormone replacement therapy may be included, provided they are clinically asymptomatic, and their thyroid hormone replacement dose has remained stable for ≥two months before the Screening Visit
u. Have obesity induced by other endocrinologic disorders (e.g., Cushing syndrome, acromegaly, inadequately treated hypothyroidism) or diagnosed monogenetic or syndromic forms of obesity (i.e., melanocortin 4 receptor deficiency or Prader Willi syndrome).
v. Have a history or diagnosis of active or unstable major depressive disorder within two years of the Screening Visit, or any history/diagnosis of other severe psychiatric conditions (e.g., schizophrenia; bipolar disorder; eating disorder; other serious mood, anxiety, or hyperactivity disorder). NOTE: Patients with depression or anxiety whose disease state, in the opinion of the Investigator, is considered stable and expected to remain stable throughout the course of the study, may be considered for inclusion only if they are adequately controlled by stable doses of selective serotonin reuptake inhibitors other than paroxetine for ≥six months before the Screening Visit, and expected to continue at the same dose for the entire study duration. Participants maintained on other types of antidepression/mood stabilizers are excluded from this study as many are associated with body weight changes.
w. Have a Patient Health Questionnaire-9 (PHQ-9) score of ≥15 at the Screening Visit or Day 1.
x. Have any lifetime history of a suicidal attempt, or suicidal behavior within 30 days before the Screening Visit.
y. Suicidal ideation on the Columbia Suicide Severity Rating Scale (C-SSRS) assessed at the Screening Visit or Day 1 and determined as:
   i. A “yes” to either Question 4 (Active Suicidal Ideation with Some Intent to Act, Without Specific Plan) OR
   ii. A “yes” answer to Question 5 (Active Suicidal Ideation with Specific Plan and Intent) on the “Suicidal Ideation” portion of C-SSRS OR
   iii. A “yes” answer to any of the suicidal-related behaviors (Actual Attempt, Interrupted Attempt, Aborted Attempt, Preparatory Act or Behavior) on the “Suicidal Behavior” portion of the C-SSRS AND
   iv. The ideation or behavior occurred within the past month.
z. Have a history of any hematologic conditions that may interfere with HbA1c measurement (e.g., hemolytic anemias, sickle cell disease, other hemoglobinopathies).
aa. Have a family or personal history of medullary thyroid carcinoma or a genetic condition that predisposes to medullary thyroid carcinoma (i.e., thyroid C-cell hyperplasia, or multiple endocrine neoplasia Type 2A or Type 2B).
bb. Have an active or untreated malignancy or have been in remission from a clinically significant malignancy (other than basal or squamous cell skin cancer, in situ carcinomas of the cervix, or in situ prostate cancer) for <five years before the Screening Visit.
cc. Have had a transplanted organ or are awaiting an organ transplant (corneal transplants [keratoplasty] may be allowed).
dd. Have any major medical and/or psychiatric conditions not indicated above, which in the opinion of the Investigator, would interfere with the study assessments, study completions, or safe treatment.
ee. Have a history of atopy (severe or multiple allergic manifestations) or clinically significant multiple or severe drug allergies, or intolerance to topical corticosteroids, or severe hypersensitivity reactions.
ff. Have a history of alcohol addiction or tendency to abuse alcohol as judged by the Investigator, and/or Alcohol Use Disorders Identification Test (AUDIT) score >14 as assessed during the Screening Visit.
gg. Have history of use of known drugs of abuse within 12 months of the Screening Visit and/or have a positive test for drug use at the Screening Visit; and/or have a history of use of marijuana or any other cannabis-related product(s) within three months of the Screening Visit and is unwilling to abstain from such product(s) during the trial. Participants must also refrain from use of cannabidiol oil for the entire duration of the study.
hh. Electrocardiogram (ECG) findings at the Screening Visit and Day 1:
   i. Indicative of active cardiac disease or clinically significant variants/abnormalities as determined by the Investigator that may interfere with the interpretation of ECG changes over the study duration
   ii. Corrected QT (QTc; Fridericia) interval ≥450 msec for male participants and ≥470 msec for female participants, or
   iii. Resting heart rate<45 bpm or >100 bpm, or
   iv. Any other significant pathologic heart rhythms; participants with first-degree block are permitted.
ii. Any of the following findings present during the Screening Period:
   i. Alanine aminotransaminase (ALT), or aspartate transaminase (AST), or gamma glutamyl transpeptidase (GGT)>5.0× the upper limit of normal (ULN) for the reference range
   ii. Alkaline phosphatase>2.0×ULN for the reference range
   iii. Total bilirubin>1.5×ULN for the reference range
   iv. Amylase or lipase>3×ULN for the reference range
   v. Calcitonin≥20 ng/L, if eGFR≥60 mL/min/1.73 m² or ≥35 ng/L if eGFR<60 mL/min/1.73 m²
jj. Estimated glomerular filtration rate (eGFR)<45 mL/min/1.73 m² by the 2021 Chronic Kidney Disease Epidemiology (CKD EPI) creatinine equation
kk. Fasting plasma triglycerides 500 mg/dL
ll. Acute or chronic viral hepatitis, determined based on hepatitis B surface antigen or the presence of hepatitis C virus RNA
mm. Have current or within six months before the Screening Visit or plan to be treated during the study duration with any treatment that may cause weight gain. These include but are not limited to tricyclic antidepressants, atypical antipsychotics, mood stabilizers, and testosterone preparations.
nn. Have started or significantly changed the dose of antihyperlipidemic medications within one months prior to the Screening Visit
oo. Are receiving or have received chronic (>14 consecutive days) systemic glucocorticoid therapy within three months prior to the Screening Visit; or have evidence of a significant, active autoimmune abnormality (eg, lupus or rheumatoid arthritis) that has required within three months prior to the Screening Visit, or is likely to require, in the opinion of the Investigator, concurrent treatment with systemic glucocorticoids during the study duration.

### Test Product(s), Dose, and Mode of Administration

CT-388 Injection, 20 mg/mL and placebo will be supplied in 1.0 mL vials. CT-388 will be administered via syringe as a single SC injection.

Participants are not allowed to self-administer the study drug at any time during this study due to the nature of the study drug preparation. Hence, participants will be required to come to the study site/clinic every week for dosing throughout the study, at approximately the same time of day and day of the week to the extent possible. Study drug will be administered by qualified study staff as a SC injection in the abdomen. The use of in-home nursing visits to administer the study drug to participants weekly may also be utilized if and when applicable.

### Duration of Treatment

Total duration of individual participation will be ˜58 weeks, including a five-week Screening Period (Screening: Days −35 to −15 and a 2-week Lead-In Period, [Days −14 to −1]), a 48 week Treatment Period (Day 1 up to Week 48) and a 5-week Safety Follow-up Period (Week 48 to Week 53).

### Efficacy Endpoints

The primary efficacy endpoints are percent change in body weight from baseline to Week 48 and change in HbA1c from baseline to Week 48.

The key secondary efficacy endpoints are body weight reduction≥5% from baseline to Week 48 (Yes/No) and HbA1c reduction to ≤7% from baseline to Week 48 (Yes/No).

Other Secondary Efficacy Endpoints are
a. Percent change in body weight from baseline to Week 28
b. Body weight reduction≥10% from baseline to Week 48 (Yes/No)
c. Body weight reduction≥15% from baseline to Week 48 (Yes/No)
d. Body weight reduction≥20% from baseline to Week 48 (Yes/No)
e. Body weight reduction≥25% from baseline to Week 48 (Yes/No)
f. Absolute change in body weight (kg) from baseline to Week 28 and Week 48
g. Percent change in body weight from baseline to Week 28 and Week 48 by obesity class
h. Change in HbA1c from baseline to Week 12, Week 28, and Week 36
i. Change in HbA1c from baseline to Week 12, Week 28, Week 36, and Week 48 by obesity class
j. HbA1c≤6.5% at Week 12, Week 28, Week 36, and Week 48 (Yes/No)
k. HbA1c<5.7% at Week 12, Week 28, Week 36, and Week 48 (Yes/No)
l. Change in 7-point SMBG profile at Week 12, Week 28, Week 36, and Week 48
m. Percentage of participants who achieve HbA1c≤6.5% and >10% weight reduction at Week 28 and Week 48
n. Percentage of participants who achieve HbA1c<7% and >5% weight reduction at Week 28 and Week 48
o. Change from baseline to Week 48 in body weight-related parameters listed below:
   i. BMI (kg/m²)
   ii. Waist circumference (cm), hip circumference (cm), and waist-to-hip ratio
   iii. Waist-to-height ratio
p. Change from baseline to Week 12, Week 28, Week 36 and Week 48 in markers of glucose metabolism:
   i. Fasting plasma glucose
   ii. Fasting insulin
   iii. Fasting C-peptide
   iv. Fasting Homeostatic Model Assessment for Insulin Resistance (HOMA-IR)
v. Quantitative insulin sensitivity check index (QUICKI)

The pharmacokinetic endpoints are CT-388 PK and metabolite concentrations.

The safety endpoints are:
a. Number of participants with ≥1 treatment-emergent adverse event (TEAE), or any serious adverse events (SAEs), adverse events (AEs) of special interest, and AEs leading to study drug discontinuation
b. TEAEs that occurred in >10% of randomized participants
c. Number of participants with documented hypoglycemia Section 10.3 (Level 1, 2, 3, per ADA 2024)
d. Number of participants with severe persistent hyperglycemia
e. Percentage of participants who required rescue therapy for T2DM management
f. Changes from baseline in C-SSRS and PHQ-9
g. Change in safety laboratory values, vital signs, and ECGs

### Antihyperglycemic Medication Use

Randomized participants are expected to continue their antihyperglycemic medication, at the same dose as at the time of randomization.

Eligible participants will have management of their T2DM with diet and exercise alone or will be on stable treatment with metformin, or metformin with or without a SGLT-2 inhibitor, for three months.

The introduction of new antihyperglycemic medications other than study drug, or medications prescribed at the time of randomization is allowed during the study ONLY as a rescue therapy to manage severe persistent hyperglycemia.

### Diet and Exercise Counseling

All randomized participants in the study will receive lifestyle management counseling with a dietician, or equivalently qualified delegate, according to local standards, on a monthly basis and at least every 12 weeks during the study, either at scheduled visits or via phone call as appropriate. Diet and exercise goals established during the lifestyle consultation and the importance of adherence to the lifestyle component of the trial will be reinforced during each contact by study staff. Participants will be instructed on how to capture their physical activity and food intake in study-provided paper diaries to facilitate review and will be provided appropriate guidance during their study visits.

Dietary counseling will consist of advice regarding healthy food choices and focus on calorie restriction using a hypocaloric diet with macronutrient composition of:
a. Maximum 25% of energy from fat
b. ˜25% of energy from protein
c. ˜50% of energy from carbohydrates
d. An energy deficit of ˜500 kcal/day compared with the participant's estimated total energy expenditure is recommended.

Total energy expenditure will be calculated using the Body Weight Planner per the National Institute of Diabetes and Digestive and Kidney disease, which will take into account the participant's weight and level of physical activity.

Participants with significant suppression of appetite must be counselled to consume at least 900 kcal/day, with the emphasis on an adequate protein intake; meal replacements such as with high protein Ensure/Boost may also be offered.

Regular physical activity (150 minutes of physical activity per week, i.e., walking) is encouraged.

The hypocaloric diet should be continued after randomization and throughout the treatment period.

If a BMI≤22 kg/m² is reached, the recommended energy intake should be recalculated with no kcal deficit for the remainder of the trial.

This guidance should be considered to be a recommendation and could be altered per the participant's needs.

To encourage adherence to this guidance, it is recommended that a three-day diet and exercise diary be completed before each counseling visit. The mobile app or a paper diary may be used. During each visit, the participant's diet is reviewed and advice to maximize adherence will be provided if needed.

### Patient Health Questionnaire-9

The PHQ-9 is a tool for assessing the severity of depression. Participants will respond to each of 9 questions, graded on a scale of 0 to +3, with 0 for not at all, +1 for several days, +2 for more than half the days, and +3 for nearly every day. Scores are totaled; a score of 1-4 indicates minimal depression, 5-9 indicates mild depression, 10-14 indicates moderate depression, 15-19 indicates moderately severe depression, and 20-27 indicates severe depression.

### Columbia-Suicide Severity Rating Scale

The C-SSRS is a tool to evaluate the severity of suicidal ideation and behavior.

### Weight and Body Mass Index

Body weight measurements should be performed in a consistent manner using a calibrated electronic scale capable of measuring weight in kilograms (kg) to 1 decimal place.

Body weight measurements should be conducted by qualified staff that are fully trained on the study procedure.

All weights for a given participant should be measured using the same scale, whenever possible, at approximately the same time in the morning after evacuation of bladder contents.

Body weight must be measured after a 28-hour fast. If the participant is not fasting, they should attend another visit within the allowable visit window for measurement of fasting body weight.

BMI will be calculated using the height measured at baseline (Lead-in Visit) and the most recent weight measurement.

### Waist and Hip Circumference

Waist circumference should be measured in the horizontal plane and at the midpoint between the lower margin of the last palpable rib and the top of the iliac crest.

Measurements should be taken at the end of a normal expiration using a non stretchable measuring tape. The tape should lie flat against the skin without compressing the soft tissue.

The waist circumference should be measured twice, rounded to the nearest 0.5 cm. The measuring tape should be removed between measurements. The mean of the 2 measurements will be recorded in the eCRF. If the difference between the 2 measurements exceeds 1 cm, this set of measurements should be discarded and the 2 measurements repeated.

### Vital Signs

Vital signs including temperature, blood pressure, heart rate, and respiratory rate will be measured at each study visit. Vital signs measurement will be conducted after the participant has rested for ≥5 minutes in a seated, supine, or semi-recumbent position.

For each parameter, including heart rate, three measurements will be taken using the same arm, preferably the nondominant arm and the recordings should be taken≥1 minute apart. If possible, blood pressure must be taken with an automated blood pressure instrument. If blood pressure and pulse measurements are taken separately, pulse should be taken before blood pressure. If pulse measurements cannot be taken via an automated blood pressure instrument, the radial artery should be used.

Remind the participant to avoid caffeine, smoking, and physical activity within 1 hour before the measurement of vital signs. The average of the three measurements will be entered into the eCRF.

### Self-Monitoring of Blood Glucose

Participants will be instructed to test their fasting glucose using the study-supplied glucometer and supplies a minimum of three times per week, and as needed based on symptoms.

Self-monitoring of blood glucose utilizing a study-provided glucometer will be initiated at Lead In and continue over the study duration.

On specific timepoints during the study, 7-point SMBG profiles consisting of measurements will be obtained before each meal, approximately two hours after each meal, and at bedtime on a day during the week before the specified scheduled clinic visits. The complete 7-point profile must be collected on a single day. If a participant does not complete the entire profile on a single day, all seven points must be collected on a subsequent day.

### Clinical Laboratory Evaluations

Blood and urine samples will be collected for hematologic, biochemical, and other clinical laboratory testing.

Of particular importance, samples for glucose and HbA1c must be collected for monitoring of safety and for the determination of the objectives of the study.

A variety of biomarkers that are thought to be influenced by obesity and its comorbidities, and those that impact cardiovascular risk factors will be assessed, and blood samples collected for their assessment.

In addition to these biomarkers, other relevant biomarkers may also emerge during the conduct of the study. Should this occur, these other biomarkers may also be assessed.

All laboratory assessments will be performed centrally. In emergency situations, local assessment of select laboratory tests may be required for urgent decision making; however, the Investigator is strongly encouraged to draw duplicate lab samples at the time of local draws and ship the duplicate samples to the central laboratory for analysis. If a participant lives in a remote area, laboratory testing may be performed locally for select visits as a last resort.

### Magnetic Resonance Imaging

MRI will be performed at selected sites. All participants randomized at these sites will participate in MRI data collection.

The MRI examination will include assessments of body composition (lean body mass/volume, visceral and subcutaneous adipose tissue volumes) and muscle composition (thigh muscle volume, contractile thigh muscle volume, intramuscular fat fraction and volume). Liver and pancreas fat fractions and liver, pancreas and spleen volumes will also be assessed. Brain perfusion will be investigated via arterial spin labeling.

No contrast agents will be administered for any MRI assessment. Participants are asked to observe fasting and to avoid hemodynamic stimulants (e.g., caffeinated drinks and medications such as acetazolamide) for four hours prior to MRI examinations. Smoking is also not permitted.

### Study Drug

CT-388 Injection, formulated to 20 mg/mL with sodium phosphate buffer and mannitol at pH 7.0 or CT-388 Placebo is supplied as a clear liquid in glass vials for SC administration. CT-388 Placebo is the same formulation without the active CT-388 ingredient. Each vial is filled with 1.0 mL of liquid for single use. All excipients are compendial. The drug product was manufactured, packaged, and labeled under current Good Manufacturing Practice regulations.

Study drug should be stored frozen at −20° C. in a secure, controlled-access location.

Study drug will only be administered by qualified study staff as a SC injection in the abdomen QW. Participants are not allowed to self-administer the study drug at any time during this study. Hence, participants are expected to come to the study site/clinic every week for administration of drug, whenever possible.

Abdominal injections of study drug should be administered at approximately the same time and day of the week, to the extent possible, throughout the study.

The use of in-home nursing visits to administer the study drug weekly to participants may also be utilized if and when applicable. The dose will be administered as a single SC injection.

The last time point to up-titrate the dose to the randomized target dose is Week 27. The last dose will be administered on Week 47.

### Dose Titration

Dosage for each participant in CT-388 low- to high-dose treatments will be escalated as shown in FIG. 21. Participants who experience GI-related symptoms should be managed per FIG. 20.

Participants must tolerate a minimum of 4 mg to continue the study drug. Dose down-titration below 4 mg will not be allowed. Participants who do not tolerate the 4-mg dose, despite symptomatic relief measures or dose modifications, including drug holidays, will be discontinued from study drug.

Dose escalations may occur at or after, but not before, the designated time for up-titration.

The last up-titration/re-challenge time point is Week 27.

Participants must tolerate a minimum of 4 mg to continue study drug. Dose down-titration below 4 mg will not be allowed and participants who do not tolerate the 4-mg dose despite symptomatic relief measures or dose modifications, including drug holidays, will be discontinued from study drug.

### Adverse Events

An AE is any unfavorable and unintended sign including an abnormal laboratory finding, symptom, or disease temporally associated with the use of a medical treatment or procedure that may or may not be considered related to the medical treatment or procedure.

TEAEs are defined as AEs that worsen or commence on or after first study drug administration. All AEs regardless of severity or how identified (e.g., volunteered, elicited, noted on physical examination) will be recorded throughout the study (i.e., from Screening until the last Follow-up Visit or 45 days after the last dose of study drug, whichever is later).

Participants will be followed for resolution of AEs, by querying the participants for an ongoing AE until the earlier of AE resolution or the last study visit. At the last study visit, all AEs require an outcome (resolved or ongoing) which will be recorded on the participant's eCRF.

Pregnancy, in and of itself, is not regarded as an AE or SAE unless the birth results in a congenital anomaly/birth defect or there is suspicion that the study drug may have interfered with the effectiveness of a contraceptive medication or method. Still, any pregnancy must be reported to Clinical Safety within 24 hours of knowledge of the event. Administration of study drug will be discontinued upon identification of a pregnancy. The pregnancy will be followed to term and/or outcome and this outcome must be reported to the pharmacovigilance team. If the outcome of the pregnancy meets the criteria for immediate classification as an SAE (i.e., postpartum complications, spontaneous abortion, stillbirth, neonatal death, or congenital abnormality), the Investigator should follow the procedures reporting an SAE.

The Investigator will rate the severity/intensity of each AE. Investigators may reference the National Cancer Institute-Common Terminology Criteria for Adverse Events (CTCAE), Version 5.0. The CTCAE displays Grades 1 through 5 with unique clinical descriptions of severity for each AE. A general grading (severity/intensity) scale, for those events not listed specifically, is provided at the beginning of the CTCAE reference document. If a particular AE's severity/intensity is not specifically addressed, the Investigator is to revert to the general scale and use his or her best medical judgment.

The five general grades are:
a. Grade 1: Mild
b. Grade 2: Moderate
c. Grade 3: Severe
d. Grade 4: Life-threatening or disabling
e. Grade 5: Death related to AE

The term “nausea” should only be used to describe the AE if the participant specifically reports a queasy sensation and the urge to vomit. The term “diarrhea” should only be used to describe the AE if the participant specifically reports passage of 3 or more watery or liquid stools per day. The Bristol stool chart may be used as a guidance to characterize diarrhea.

The Investigator will make a blinded determination of the relationship of the AE to the study drug (including placebo) using a 5-category system (not related, unlikely, possible, probable, and definite).

A serious adverse event (SAE) is any untoward medical occurrence at any dose that results in any of the following outcomes:
a. Death
b. A life threatening event (i.e., places the participant, in the view of the Investigator, at immediate risk of death)
c. Inpatient hospitalization or prolongation of existing hospitalization
d. Any admission or hospitalization involving an overnight stay.
e. An emergency room or urgent care visit without formal inpatient hospital admission will not be recorded as an SAE under this criterion, nor will hospitalization for a procedure scheduled or planned before signing of informed consent. However, unexpected complications and/or prolongation of hospitalization that occur during elective surgery should be recorded as AEs and assessed for seriousness.
f. A persistent or significant incapacity or substantial disruption of the ability to conduct normal life functions
g. A congenital anomaly/birth defect

Important medical events that may not result in death, be life-threatening, or require hospitalization may be considered SAEs when, based upon appropriate medical judgment, they may jeopardize the participant and may require medical or surgical intervention to prevent one of the outcomes listed in this definition.

An unexpected adverse drug event is any adverse drug event the specificity or severity of which is not consistent with the current Investigator's Brochure. SAEs that are unexpected and related are reportable to Regulatory Authorities. Reportable SAEs will be reported by the Investigator to the Sponsor and where required the responsible EC or IRB. To report an SAE, site personnel will complete and submit an SAE form within 24 hours of becoming aware of the event to the Sponsor's representative.

### Abnormal Laboratory Findings and Other Abnormal Findings

Abnormal laboratory findings and other abnormal investigational findings (eg, on an ECG trace) should not be reported as AEs unless they are associated with clinical signs and symptoms, lead to treatment discontinuation, or are considered otherwise medically important by the Investigator. If an abnormality fulfills these criteria, the identified medical condition (e.g., anemia, increased ALT) must be reported as the AE rather than the abnormal value itself. The Investigator will rate the severity of laboratory abnormal findings per the National Cancer Institute-Common Terminology Criteria for Adverse Events (CTCAE), Version 5.0.

### Precautions

Participants who experience GI symptoms (e.g., nausea, vomiting, or diarrhea) at any time during the study should first be counseled on dietary behaviors that may help mitigate nausea and vomiting, (e.g., eating smaller-size portions, splitting 3 daily meals into 4 or more smaller ones, and stopping eating when they feel full).

If symptoms persist, the participant should be prescribed, at the Investigator's discretion, symptomatic medication (e.g., anti-emetic or antidiarrheal medication) (FIG. 20).

Indigestion or abdominal bloating symptoms during the study may be treated symptomatically with agents that do not increase QTc interval (eg, calcium carbonate, bismuth subsalicylate). H2 receptor antagonists (e.g., famotidine, known to prolong QTc interval) are contraindicated.

Nausea and/or vomiting during this study may be treated with antiemetics such as ondansetron but should not be used prophylactically. Smaller-sized meal portions and other conservative measures should be encouraged to mitigate nausea/vomiting, which may be seen as a consequence of delayed/slowed gastric emptying, the latter a known PD effect of the GLP-1 and incretin-based drug class.

Constipation should initially be treated with hydration, fiber, and eating smaller-size portions. If medication is needed, Coloxyl with senna or Movicol, Metamucil, Colace or Dulcolax or regional equivalents are recommended.

Diarrhea, only if considered severe and significantly impacting the participant's ability to function, may be treated with short duration (1-2 days) of loperamide. Opioid-based drugs are generally prohibited for use during this study given their well-established impact in reducing GI motility, which in turn may further exacerbate GI-related AEs.

A temporary interruption of study drug for one dose due to GI symptoms is permitted, provided the participant has taken the last 3 weekly doses. Study treatment should be resumed at the assigned dose immediately, either alone or in combination with symptomatic medication, which can also be utilized to manage symptoms.

If intolerable GI symptoms (e.g. nausea, vomiting, or diarrhea) persist despite the above measures, the Investigator may consider re-initiating study drug at the next-lowest maintenance dose in a blinded fashion (e.g., 24 mg reduced to 20 mg, 20 mg to 16 mg, 16 mg reduced to 12 mg, 12 mg reduced to 8 mg, or 8 mg reduced to 4 mg) (FIG. 20). If intolerable GI symptoms persist despite symptomatic treatment, temporary drug interruption, and resumption at a lower dose of study drug, the participant should be discontinued from study drug. All participants who discontinue study drug should continue to attend scheduled study visits.

1. A method of
managing weight,
improving glycemic control, and/or
increasing insulin sensitivity
in an overweight or obese adult patient in need thereof, comprising administering by subcutaneous injection to the patient a compound of Formula I, or a pharmaceutically acceptable salt or ester thereof: 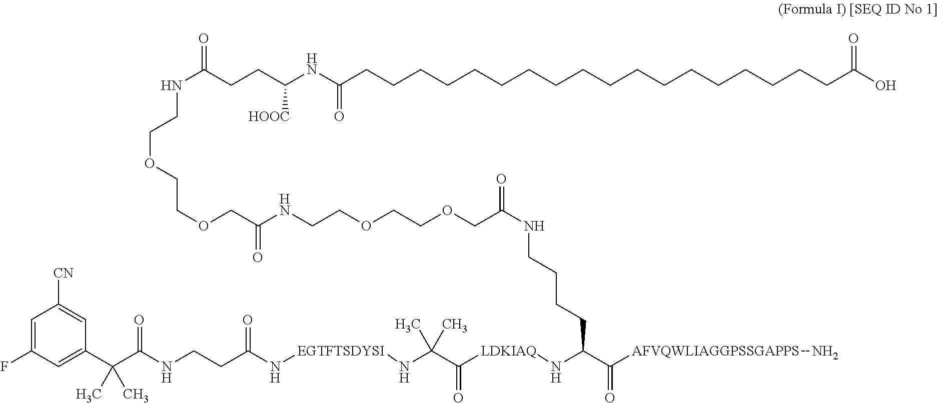
wherein the compound is administered at a body weight-independent dose of about 2.0 mg to about 25 mg.

2. The method of claim 1, wherein the patient has a BMI of ≥25 kg/m², ≥27 kg/m², ≥30 kg/m², ≥35 kg/m², or ≥40 kg/m².

3. The method of claim 1, wherein the patient is obese.

4. The method of claim 3, wherein the patient does not have type 2 diabetes mellitus, or is prediabetic.

5. The method of claim 3, wherein the patient has type 2 diabetes mellitus.

6. The method of claim 1, wherein the patient has one or more weight-related comorbidities, optionally prediabetes, hypertension, or dyslipidemia.

7. The method of claim 1, wherein the administering step is repeated once every week, once every two weeks, once every four weeks, or once every month.

8. The method of claim 1, wherein the dose is about 5, 6, 7.5, 8, 12, 16, 17, 22, or 24 mg.

9. The method of claim 1, wherein the dose is up-titrated over a period of 3 weeks to 30 weeks.

10. The method of claim 9, wherein the starting dose for the up-titration is about 5 mg or about 8 mg.

11. The method of claim 10, wherein the maximum dose for the up-titration is about 22 or 24 mg.

12. The method of claim 1, wherein the patient receives an additional therapy for overweight or obesity, optionally wherein the additional therapy is diet therapy or exercise therapy.

13. The method of claim 1, wherein administration of the compound results in reduction of visceral adiposity and/or HbA1c level in the patient.

14-15. (canceled)

16. An article of manufacture comprising a body weight-independent dose of about 2.0 mg to about 25 mg of a compound of Formula I, or a pharmaceutically acceptable salt or ester thereof:

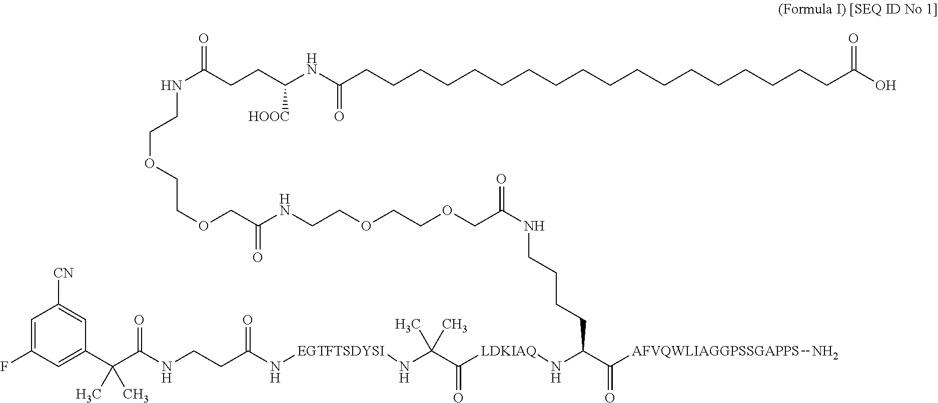

17. The article of manufacture of claim 16, wherein the article of manufacture comprises a syringe or an injector, optionally a single-use syringe or injector.
